SEQUENCE LISTING (49 sequences)

SEQ ID NO: 1 - DNA, 12166 bp, Escherichia coli
ctgaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacccgattggggcgtggacataagggtctttatgacacaatcaacaattcgcttcattttcaattaggccttgctctagcttctttaggggttattacttctttggtagctcaacacatgtactctttacctgcttatgcattcatagcacaagactttactactcaagctgcattatatacccaccaccaatatatcgcaggattcatcatgacaggagcttttgctcatggagctatatttttcattagagattacaatccggagcaaaatgaagataatgtattggcaagaatgttagatcataaagaagctatcatatctcatttaagttgggccagcctctttctgggattccataccctgggactttatgttcataatgatgtcatgcttgcctttggcactccggagaagcaaatcttgattgaaccgatatttgctcaatggatacaatccgctcatggtaaaacttcatatgggttcgatgtacttttatcttcaacgactggcccagcattcaatgcgggtcgaagcatctggttgccgggttggttaaatgctgttaatgaaaatagtaattcattattcttaacaataggtcctggagactttttggttcatcatgctattgcccttggtttacatacaactacattgatcttagtaaaaggtgctttagatgcacgtggttccaagttaatgccagataaaaaggatttcggttatagttttccgtgcgatggcccaggacgaggcggtacttgtgatatttcggcatgggacgcgttttatttggcagttttttggatgttaaatactattggatgggttactttttattggcattggaagcacatcacattatggcaaggtaacgtttcacagtttaatgaatcttccacttatttgatgggctggttaagggattatttatggttaaactcttcacaacttatcaatggatataacccttttggtatgaatagtttatcggtttgggcatggatgttcttatttggacatcttgtttgggctactggatttatgttcttaatttcttggcgtggatattggcaggaattgattgaaactttagcatgggctcacgaacgcacacctttggccaatttgattcgatggagagataaaccagtggccctttctattgtacaagcaagattggttggattagctcacttttctgtaggttatatattcacttatgcggctttcttgattgcctctacgtcgggcaaatttggttaattaatgtgtgtattcgcgataatctcatttctttcgacggagaagggggtccaccttcttctatttctacatctaggattcgacttgtatcatggatactaataggaattcaaccattatggcaaggaaaagtttgattcagagggagaagaagaggcaaaaattggaacagaaatatcattcgattcgtcgatcctcaaaaaaagaaataagcaaggttccgtcgttgagtgacaaatgggaaatttatggaaagttacaatccctaccacggaatagtgcacctacacgccttcatcgacgttgttttttgaccggaaggccgagagctaactatcgagactttggcctatccggacacatacttcgtgaaatggttcatgcatgtttgttgccaggagcaacaagatcaagttggtaaggattaacgcttcatttctatttctatggtcgatgatcatagaagcccctttaccattctgtataaatgggctattctatttgtacagatagggtggaggggcgcatttaatccttgtttatctattagttttcagttcttatctttggcgcggggtagagcagtttggtagctcgcaaggctcataaccttgaggtcacgggttcaaatcctgtctccgcaacatcttattctggtctcgaggtccgcataaagaaccacccataatacccataatagctgtttgccaaccggtcgccgtcgttcaatgagaatggataagaggctcgtgggattgacgtgagggggcagggatgactatatttctgggagcgaactccgggcgaatatgaagcgcatggatacaagttatgccttggaatgaaagacaattccgaatccgctttgtctacgaacaaggaagctataagtaatgcaactatgaaggatctatgactgacgaaaacattagacagatagctttctacggtaaaggcggtatcggtaaatctaccacctcccaaaacacccttgcagctatggcagaaatgggtcaacgcatcatgattgtaggttgcgaccctaaagctgactccacccgtctgatgcttcactccaaagctcaaaccaccgtactacacttagctgctgaacgcggtgcagtagaagacttagaactccacgaagtaatgttgaccggtttccgtggcgttaagtgcgtagaatctggtggtccagaacccggtgtaggttgcgccggtcgtggtatcatcaccgccattaacttcttagaagaaaacggcgcttaccaagacctagacttcgtatcctacgacgtattgggtgacgttgtatgtggtggtttcgctatgcctatccgtgaaggtaaagcacaagaaatctacatcgttacctctggtgaaatgatggcgatgtatgctgctaacaacatcgctcgcggtattttgaaatatgctcactccggtggtgtacgtttaggtggtttgatctgtaacagccgtaaggttgaccgtgaagacgagttaatcatgaacttggctgaacgtttgaacacccaaatgattcacttcgtacctcgtgacaacatcgttcaacacgcagaattgcgccgtatgaccgttaacgagtacgcaccagacagcaaccaaggtcaagagtaccgcgcattagctaagaagatcatcaacaacgacaagctcaccattcctacaccaatggaaatggatgaactagaagctctgttgatcgaatacggtctattagacgacgacaccaagcactctgaaatcatcggtaagcccgcagaagctaccaaatagcggccgcaacccataatacccataatagctgtttgccatcgctaccttaggaccgttatagttaaccggtggaggcagactatgacacctcctgaaaacaagaatcttgtagatgaaaataaggaacttattcaagaagttctgaaagcttatcccgaaaaatctcgcaaaaagcgcgaaaaacacctcaacgtccacgaagaaaacaagtctgattgcggcgttaagtctaacatcaaatccgttcctggtgtaatgaccgcccgtggttgtgcttatgcaggttctaagggtgtggtttggggtcctattaaggacatgatccacatcagccacgggcctgtaggttgcggttactggtcttggtctggtcgtcgtaactactacgttggtgtaactggtatcaactctttcggtaccatgcacttcacctcagacttccaagaacgtgacatcgtgttcggtggtgacaaaaaactcactaaactcattgaagaactcgatgttcttttccctctcaaccgtggtgtttccattcaatctgaatgtcccattggtctaattggggatgacatcgaagctgttgctaagaaaacttctaagcaaattggtaagcctgttgtacccttacgttgcgaaggtttccgtggtgtgtctcagtccttaggacaccacatcgctaacgacgctatccgtgactggattttcccagaatacgacaagctcaagaaagaaaacagacttgacttcgagccaagcccctatgatgtagctctaatcggtgactacaacatcggtggtgacgcttgggccagccggatgctgttggaagaaatgggcttacgtgtagtagctcagtggtctggtgatggtacactcaacgagttgatccaaggccctgctgctaagttagtcctcatccactgctaccgttctatgaactacatctgccgtagtttggaagaacaatacggtatgccttggatggagttcaacttcttcggccccaccaagattgctgcttctttacgtgaaatcgcagctaagtttgattctaagattcaagaaaacgctgagaaggtaattgctaagtacacaccagtaatgaatgctgtactagataaataccgccctcgcttggaaggtaacaccgtaatgttgtacgtaggtggtctacgtcctcgccacgttgttcccgcttttgaagacctgggtatcaaagtagttggtactggctatgaattcgctcacaatgacgattacaaacgtaccacccactacatcgataacgccaccatcatttacgatgacgttaccgcctacgaatttgaagagttcgtaaaagctaagaagcctgatttaattgcttctggtattaaagagaagtacgtcttccaaaagatgggtcttcccttccgtcaaatgcactcttgggattactccggcccttatcacggttacgacggattcgctatcttcgcccgtgacatggatttagccctcaacagcccaacttggagcttgattggcgctccttggaagaaagcggctgcaaaggctaaggctgctgcctaactcgagagatacaacaatgcctcagaatccagaaagaactgtagaccacgttgatctattcaaacagccagaatacaccgagctatttgaaaacaagagaaagaactttgaaggcgctcatcctcctgaagaagttgaaagagtgtctgaatggacaaaatcttgggactaccgggaaaagaacttcgctcgtgaagctttaaccgttaaccctgctaaaggttgccaacctgtaggcgcgatgttcgctgctttgggttttgaaggtactctacctttcgtacaaggttctcaaggttgcgttgcttacttccgtacacacctcagccgtcactacaaagagccttgctccgcagtatcttcttccatgacagaagatgcagcagtattcggtggtttgaacaacatgatcgaaggtatgcaggtttcataccaactgtacaagcctaagatgattgctgtttgcaccacctgtatggcggaagttatcggagatgacttgggcgcgttcatcaccaactccaagaacgctggttctattcctcaagatttccccgtaccctttgctcacacacctagctttgttggttcccacatcactggctacgacaacatgatgaagggtattctgtctaacttgacagaaggtaagaagaaagctaccagcaacggcaaaattaacttcattcctggttttgatacctatgtaggtaacaaccgcgaattgaagcggatgatgggtgtaatgggtgttgactacaccatcctgtctgacagcagcgactactttgattcacctaacatgggtgaatacgaaatgtacccaggtggtacaaagctggaagatgcggctgattctatcaacgctaaagcaactgttgctctccaagcttacaccacacctaagacccgcgaatacatcaaaacccagtggaagcaagaaacacaagtattgcgccccttcggtgttaagggtactgacgagttcttgactgctgtttctgaattgaccggtaaagctattcctgaagaattggaaatcgaacgcggtcgtttagttgatgctatcactgactcctacgcttggattcatggtaagaagttcgctatctacggcgatccagatttgatcatctccatcaccagcttcttgttagaaatgggtgctgaaccagtacacatcctctgcaacaacggtgatgacaccttcaagaaagaaatggaagctatcctcgctgctagcccatttggtaaagaagctaaagtctggattcaaaaagacttgtggcacttccgttccttgttgttcaccgagcctgtagacttcttcatcggtaactcctacggtaagtacctgtggcgcgataccagcatcccaatggtgcggattggttatcctctcttcgatcgccaccacttacaccgctattctaccctcggctaccaaggtggtctaaatatcctcaactgggttgttaacaccctgttggatgaaatggatcgcagcaccaacatcactggtaagaccgatatctcctttgacttgatccgctaggcggccgctcgctaccttaggaccgttatagttattaccctgttatccctaaccggtggaggcttcttgttatgacatgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatctcgagctcaagcttcgaattctgcagtcgacggtaccgcgggcccgggatccacctgatctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacgcggccgcattaccctgttatccctattaattaagagctcgctaccttaagagaggatatcggcgcgccgatcctagcctagtctataggaggttttgaaaagaaaggagcaataatcattttcttgttctgtcaagagggtgctattgctcctttctttttttttttttctttactaatttcctagtattttactgacatagacttttttgtttacattatcgaaaaagaaagagagggtatttgcttgcatttattcatgatggatcccccgggctgcaggcatgcaagctaattcccgatctagtaacatagatgacaccgcgcgccgcggaactagtaattaattcccgcctttcgctttttgggggtggaaggcaaaagaaaacgtaggggagggatagaatcactacactatcacggccaactataccaactccttaatgtaaggatatatttaatgctatttatgaaattcaataataaaaagaaatagtaaaaaaattactttatcttggatcttgggcggatagcgggaatcgaacccgcatcttctccttggcaaagagaaattttaccattcgaccatatccgcatttttttgttcttgatacacaatatgtacccacatatatgatatataaccggatcttatttgtgcagtgccgggacacatattctcttcggaacgattccaataattttgttaattatattctttttattcaagaagtttgacccccctctaatttttttgttttctttatttgatttgcattttctttggggacttagattcaaatttaatgtgtctcacaaccgagaaaaattaggggggtcattttggttttgggtctgcgacgaataggttcaagagatgagagaattaaggatacccaccagaaagactaatccaatccataaggaggtaccagaaaatacaacatttttgttacttgaccagccatcaggagaagcaaatacaacgggtacgctaatcaataagattaatgaagtagcaattaatgcaaaaacagccaattggaaagcaagagtcatgcttttaatcctccaagctaccaacaaatgaactatataccatttgatccctctatcagccaaaaaatattaattgtgataaaatatgtcatcgagggattttactttatcatgaatccattgattctatatgacttattactactccccctttcgcactttattcgtacatggagtggggtggggggaaatggaattttcttttttatttcacaaatggacatgctagatcatatatctatatacggatagatagatcgatcggcggattcgcacctgagatctttctacagatagtgggggtatccacccctatagccatgttctattcggaggaataaaataaaaatagtctttcggagagatggctgagtggttgatagccccggtcttgaaaaccggtatagttttgaacaaagaactatcgagggttcgaatccctctctctccttttttgctaattgaatagatttttttatttagtggttttgcccaatctgctatccgaaagaaaagggaatggctcggctatcccacctagccaagccagaaaaatagattagatataaattagataaaataaatgagttgaaaaaaaaaaaagaaaaaaggaatacttaagctgattccaagatgtatgattgaatcaaagtaatttgtacttcattcaagcattggatctcctgtctcatatcaattaagaggggtcatggaaagaacaggttcaaagtcgcgatcaattcctttttcaaatcctgctgcagcgagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 2 - DNA, 12166 bp, Escherichia coli
ctgaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacccgattggggcgtggacataagggtctttatgacacaatcaacaattcgcttcattttcaattaggccttgctctagcttctttaggggttattacttctttggtagctcaacacatgtactctttacctgcttatgcattcatagcacaagactttactactcaagctgcattatatacccaccaccaatatatcgcaggattcatcatgacaggagcttttgctcatggagctatatttttcattagagattacaatccggagcaaaatgaagataatgtattggcaagaatgttagatcataaagaagctatcatatctcatttaagttgggccagcctctttctgggattccataccctgggactttatgttcataatgatgtcatgcttgcctttggcactccggagaagcaaatcttgattgaaccgatatttgctcaatggatacaatccgctcatggtaaaacttcatatgggttcgatgtacttttatcttcaacgactggcccagcattcaatgcgggtcgaagcatctggttgccgggttggttaaatgctgttaatgaaaatagtaattcattattcttaacaataggtcctggagactttttggttcatcatgctattgcccttggtttacatacaactacattgatcttagtaaaaggtgctttagatgcacgtggttccaagttaatgccagataaaaaggatttcggttatagttttccgtgcgatggcccaggacgaggcggtacttgtgatatttcggcatgggacgcgttttatttggcagttttttggatgttaaatactattggatgggttactttttattggcattggaagcacatcacattatggcaaggtaacgtttcacagtttaatgaatcttccacttatttgatgggctggttaagggattatttatggttaaactcttcacaacttatcaatggatataacccttttggtatgaatagtttatcggtttgggcatggatgttcttatttggacatcttgtttgggctactggatttatgttcttaatttcttggcgtggatattggcaggaattgattgaaactttagcatgggctcacgaacgcacacctttggccaatttgattcgatggagagataaaccagtggccctttctattgtacaagcaagattggttggattagctcacttttctgtaggttatatattcacttatgcggctttcttgattgcctctacgtcgggcaaatttggttaattaatgtgtgtattcgcgataatctcatttctttcgacggagaagggggtccaccttcttctatttctacatctaggattcgacttgtatcatggatactaataggaattcaaccattatggcaaggaaaagtttgattcagagggagaagaagaggcaaaaattggaacagaaatatcattcgattcgtcgatcctcaaaaaaagaaataagcaaggttccgtcgttgagtgacaaatgggaaatttatggaaagttacaatccctaccacggaatagtgcacctacacgccttcatcgacgttgttttttgaccggaaggccgagagctaactatcgagactttggcctatccggacacatacttcgtgaaatggttcatgcatgtttgttgccaggagcaacaagatcaagttggtaaggattaacgcttcatttctatttctatggtcgatgatcatagaagcccctttaccattctgtataaatgggctattctatttgtacagatagggtggaggggcgcatttaatccttgtttatctattagttttcagttcttatctttggcgcggggtagagcagtttggtagctcgcaaggctcataaccttgaggtcacgggttcaaatcctgtctccgcaacatcttattctggtctcgaggtccgcataaagaaccacccataatacccataatagctgtttgccaaccggtcgccgtcgttcaatgagaatggataagaggctcgtgggattgacgtgagggggcagggatgactatatttctgggagcgaactccgggcgaatatgaagcgcatggatacaagttatgccttggaatgaaagacaattccgaatccgctttgtctacgaacaaggaagctataagtaatgcaactatgaaggatctatgactgatgaaaatattagacaaattgctttttatggaaaaggaggaattggaaaatctactacttctcaaaatactttagctgctatggctgaaatgggacaaagaattatgattgtaggatgtgatcctaaagctgattctactagattaatgttacattctaaagctcaaactactgtattacatttagctgctgaaagaggagctgtagaagatttagaattacatgaagtaatgttaactggatttagaggagtaaaatgtgtagaatctggaggacctgaacctggagtaggatgtgctggaagaggaattattactgctattaattttttagaagaaaatggagcttatcaagatttagattttgtatcttatgatgtattaggagatgtagtatgtggaggatttgctatgcctattagagaaggaaaagctcaagaaatttatattgtaacttctggagaaatgatggctatgtatgctgctaataatattgctagaggaattttaaaatatgctcattctggaggagtaagattaggaggattaatttgtaattctagaaaagtagatagagaagatgaattaattatgaatttagctgaaagattaaatactcaaatgattcattttgtacctagagataatattgtacaacatgctgaattaagaagaatgactgtaaatgaatatgctcctgattctaatcaaggacaagaatatagagctttagctaaaaaaattattaataatgataaattaactattcctactcctatggaaatggatgaattagaagctttattaattgaatatggattattagatgatgatactaaacattctgaaattattggaaaacctgctgaagctactaaatagcggccgcaacccataatacccataatagctgtttgccatcgctaccttaggaccgttatagttaaccggtggaggcagactatgactcctcctgaaaataaaaatttagtagatgaaaataaagaattaattcaagaagtattaaaagcttatcctgaaaaatctagaaaaaaaagagaaaaacatttaaatgtacatgaagaaaataaatctgattgtggagtaaaatctaatattaaatctgtacctggagtaatgactgctagaggatgtgcttatgctggatctaaaggagtagtatggggacctattaaagatatgattcatatttctcatggacctgtaggatgtggatattggtcttggtctggaagaagaaattattatgtaggagtaactggaattaattcttttggaactatgcattttacttctgattttcaagaaagagatattgtatttggaggagataaaaaattaactaaattaattgaagaattagatgtattatttcctttaaatagaggagtatctattcaatctgaatgtcctattggattaattggagatgatattgaagctgtagctaaaaaaacttctaaacaaattggaaaacctgtagtacctttaagatgtgaaggatttagaggagtatctcaatctttaggacatcatattgctaatgatgctattagagattggatttttcctgaatatgataaattaaaaaaagaaaatagattagattttgaaccttctccttatgatgtagctttaattggagattataatattggaggagatgcttgggcttctagaatgttattagaagaaatgggattaagagtagtagctcaatggtctggagatggaactttaaatgaattaattcaaggacctgctgctaaattagtattaattcattgttatagatctatgaattatatttgtagatctttagaagaacaatatggaatgccttggatggaatttaatttttttggacctactaaaattgctgcttctttaagagaaattgctgctaaatttgattctaaaattcaagaaaatgctgaaaaagtaattgctaaatatactcctgtaatgaatgctgtattagataaatatagacctagattagaaggaaatactgtaatgttatatgtaggaggattaagacctagacatgtagtacctgcttttgaagatttaggaattaaagtagtaggaactggatatgaatttgctcataatgatgattataaaagaactactcattatattgataatgctactattatttatgatgatgtaactgcttatgaatttgaagaatttgtaaaagctaaaaaacctgatttaattgcttctggaattaaagaaaaatatgtatttcaaaaaatgggattaccttttagacaaatgcattcttgggattattctggaccttatcatggatatgatggatttgctatttttgctagagatatggatttagctttaaattctcctacttggtctttaattggagctccttggaaaaaagctgctgctaaagctaaagctgctgcttaactcgagagatacaacaatgcctcaaaatcctgaaagaactgtagatcatgtagatttatttaaacaacctgaatatactgaattatttgaaaataaaagaaaaaattttgaaggagctcatcctcctgaagaagtagaaagagtatctgaatggactaaatcttgggattatagagaaaaaaattttgctagagaagctttaactgtaaatcctgctaaaggatgtcaacctgtaggagctatgtttgctgctttaggatttgaaggaactttaccttttgtacaaggatctcaaggatgtgtagcttattttagaactcatttatctagacattataaagaaccttgttctgctgtatcttcttctatgactgaagatgctgctgtatttggaggattaaataatatgattgaaggaatgcaagtatcttatcaattatataaacctaaaatgattgctgtatgtactacttgtatggctgaagtaattggagatgatttaggagcttttattactaattctaaaaatgctggatctattcctcaagattttcctgtaccttttgctcatactccttcttttgtaggatctcatattactggatatgataatatgatgaaaggaattttatctaatttaactgaaggaaaaaaaaaagctacttctaatggaaaaattaattttattcctggatttgatacttatgtaggaaataatagagaattaaaaagaatgatgggagtaatgggagtagattatactattttatctgattcttctgattattttgattctcctaatatgggagaatatgaaatgtatcctggaggaactaaattagaagatgctgctgattctattaatgctaaagctactgtagctttacaagcttatactactcctaaaactagagaatatattaaaactcaatggaaacaagaaactcaagtattaagaccttttggagtaaaaggaactgatgaatttttaactgctgtatctgaattaactggaaaagctattcctgaagaattagaaattgaaagaggaagattagtagatgctattactgattcttatgcttggattcatggaaaaaaatttgctatttatggagatcctgatttaattatttctattacttcttttttattagaaatgggagctgaacctgtacatattttatgtaataatggagatgatacttttaaaaaagaaatggaagctattttagctgcttctccttttggaaaagaagctaaagtatggattcaaaaagatttatggcattttagatctttattatttactgaacctgtagatttttttattggaaattcttatggaaaatatttatggagagatacttctattcctatggtaagaattggatatcctttatttgatagacatcatttacatagatattctactttaggatatcaaggaggattaaatattttaaattgggtagtaaatactttattagatgaaatggatagatctactaatattactggaaaaactgatatttcttttgatttaattagataggcggccgctcgctaccttaggaccgttatagttattaccctgttatccctaaccggtggaggcttcttgttatgacatgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatctcgagctcaagcttcgaattctgcagtcgacggtaccgcgggcccgggatccacctgatctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacgcggccgcattaccctgttatccctattaattaagagctcgctaccttaagagaggatatcggcgcgccgatcctagcctagtctataggaggttttgaaaagaaaggagcaataatcattttcttgttctgtcaagagggtgctattgctcctttctttttttttttttctttactaatttcctagtattttactgacatagacttttttgtttacattatcgaaaaagaaagagagggtatttgcttgcatttattcatgatggatcccccgggctgcaggcatgcaagctaattcccgatctagtaacatagatgacaccgcgcgccgcggaactagtaattaattcccgcctttcgctttttgggggtggaaggcaaaagaaaacgtaggggagggatagaatcactacactatcacggccaactataccaactccttaatgtaaggatatatttaatgctatttatgaaattcaataataaaaagaaatagtaaaaaaattactttatcttggatcttgggcggatagcgggaatcgaacccgcatcttctccttggcaaagagaaattttaccattcgaccatatccgcatttttttgttcttgatacacaatatgtacccacatatatgatatataaccggatcttatttgtgcagtgccgggacacatattctcttcggaacgattccaataattttgttaattatattctttttattcaagaagtttgacccccctctaatttttttgttttctttatttgatttgcattttctttggggacttagattcaaatttaatgtgtctcacaaccgagaaaaattaggggggtcattttggttttgggtctgcgacgaataggttcaagagatgagagaattaaggatacccaccagaaagactaatccaatccataaggaggtaccagaaaatacaacatttttgttacttgaccagccatcaggagaagcaaatacaacgggtacgctaatcaataagattaatgaagtagcaattaatgcaaaaacagccaattggaaagcaagagtcatgcttttaatcctccaagctaccaacaaatgaactatataccatttgatccctctatcagccaaaaaatattaattgtgataaaatatgtcatcgagggattttactttatcatgaatccattgattctatatgacttattactactccccctttcgcactttattcgtacatggagtggggtggggggaaatggaattttcttttttatttcacaaatggacatgctagatcatatatctatatacggatagatagatcgatcggcggattcgcacctgagatctttctacagatagtgggggtatccacccctatagccatgttctattcggaggaataaaataaaaatagtctttcggagagatggctgagtggttgatagccccggtcttgaaaaccggtatagttttgaacaaagaactatcgagggttcgaatccctctctctccttttttgctaattgaatagatttttttatttagtggttttgcccaatctgctatccgaaagaaaagggaatggctcggctatcccacctagccaagccagaaaaatagattagatataaattagataaaataaatgagttgaaaaaaaaaaaagaaaaaaggaatacttaagctgattccaagatgtatgattgaatcaaagtaatttgtacttcattcaagcattggatctcctgtctcatatcaattaagaggggtcatggaaagaacaggttcaaagtcgcgatcaattcctttttcaaatcctgctgcagcgagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 3 - DNA, 11875 bp, Escherichia coli
ctgaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccatatttaatatggcatcaccagaggtaccagaaatgtatttacctttcctcccaaaaacacttattttaaaatctaattattgacagaagttgaccttgtaaatattacttaaattaatttagcagtatgtcaatttagttagttatagtaccactttgggagagggtattacattaatttattttgaaatttacttatagtaaatagagatttgggaacaacaactgctagcctaccgatttacaaaacgtggttttaataatttattaattggaaggcgactgcaaaagaatattttgcagcataggatttatttctaatttttctaaagttaccaattgagttaaaaaaagctttcttaattcaacatttttaagtaaatactgtttaatgttatacttttacgaatacacatatggtaaaaaataaaacaatatctttaaaataagtaaaaataatttgtaaaccaataaaaaatatatttatggtataatataacatatgatgtaaaaaaaactatttgtctaatttaataaccatgcattttttatgaacacataataattaaaagcgttgctaatggtgtaaataatgtatttattaaattaaataattgttattataaggagaaatccatgacaattagtactccagagcgcgaagcgaaaaaagtaaagattgcggttgatcgtaatcctgtagaaacaagttttgaaaaatgggcgtgcgacctgaaataaggatttgactaattaaatataaaaatttttaactaaaataagctgaaaaagcttattttagttttaagcactgttggccgagcggatgaggcaaacgactcataatcgttataaggtaggttcaactcctatacggtgcaactaagtaaagaataagcaatacttccttttcaaaatatggatggtcataatatatgctattctggtagaatttataacaagccttttcagaagatatatatatataactttttatagtaaacttgttggagatttaaattaacatctaggagcaatcgtccactctaatgagttaatataactctaaaagctgaaagtaatccatattgttcctactccggttttctaagttatttttgttaaaggagtcgtctggtttactccaacaaaagaaattggttttattttttctatgtcatatacatgcgatagagtattagtttttaatacttgcttaaaaaaaatttttgtgatataatattattagattaagttaatgcggacatagctcaatggtagagtatttccttgccaaggaaaatgttgcgggttcgactcccgttgtccgctagaaactactcgaggtccgcataaagaaccacccataatacccataatagctgtttgccaaccggtcgcgcctgccaactgcctatatttatatactgcgataaactttagtcccgaaggggtttacatatccgaaggaggaagcaggcagtggcggtaccacgccactggcgtcctaatataaatattgggcaagtaaacttagaataaaatttatttgctgcgttagcaggtttacatactcctaagtttacttgcccgaaggggaaggaggacgtcccctacgggaatataaatattagtggcagtggtacaataaataaattgtatgtaaaccccttcgggcaactaaagtttatcgcagtattaacatcctagtatataaatatcggcagttggcaggcaacaaatttatttattgtcccgtaaggggaaggggaaaacaattattattttactgcggagcagcttgttattagaaatttttattaaaaaaaaaataaaaatttgacaaaaaaaaataaaaaagttaaattaaaaacactgggaatgttctaacaatcataaaaaaatcaaaagggtttaaaatcccgacaaaatttaaactttaaagagtatgacagatgaaaatattcgtcaaattgctttttatggtaaaggtggtattggtaaatcaacaacatcacaaaatacattagctgctatggctgaaatgggtcaacgtattatgattgttggttgtgatccaaaagctgattcaacacgtttaatgttacattcaaaagctcaaacaacagttttacatttagctgctgaacgtggtgctgttgaagatttagaattacatgaagttatgttaacaggttttcgtggtgttaaatgtgttgaatcaggtggtccagaaccaggtgttggttgtgctggtcgtggtattattacagctattaattttttagaagaaaatggtgcttatcaagatttagattttgtttcatatgatgttttaggtgatgttgtttgtggtggttttgctatgccaattcgtgaaggtaaagctcaagaaatttatattgttacatcaggtgaaatgatggctatgtatgctgctaataatattgctcgtggtattttaaaatatgctcattcaggtggtgttcgtttaggtggtttaatttgtaattcacgtaaagttgatcgtgaagatgaattaattatgaatttagctgaacgtttaaatacacaaatgattcattttgttccacgtgataatattgttcaacatgctgaattacgtcgtatgacagttaatgaatatgctccagattcaaatcaaggtcaagaatatcgtgctttagctaaaaaaattattaataatgataaattaacaattccaacaccaatggaaatggatgaattagaagctttattaattgaatatggtttattagatgatgatacaaaacattcagaaattattggtaaaccagctgaagctacaaaatagcggccgcaacccataatacccataatagctgtttgccatcgctaccttaggaccgttatagttaaccggtggaggcagactatgacaccaccagaaaataaaaatttagttgatgaaaataaagaattaattcaagaagttttaaaagcttatccagaaaaatcacgtaaaaaacgtgaaaaacatttaaatgttcatgaagaaaataaatcagattgtggtgttaaatcaaatattaaatcagttccaggtgttatgacagctcgtggttgtgcttatgctggttcaaaaggtgttgtttggggtccaattaaagatatgattcatatttcacatggtccagttggttgtggttattggtcatggtcaggtcgtcgtaattattatgttggtgttacaggtattaattcatttggtacaatgcattttacatcagattttcaagaacgtgatattgtttttggtggtgataaaaaattaacaaaattaattgaagaattagatgttttatttccattaaatcgtggtgtttcaattcaatcagaatgtccaattggtttaattggtgatgatattgaagctgttgctaaaaaaacatcaaaacaaattggtaaaccagttgttccattacgttgtgaaggttttcgtggtgtttcacaatcattaggtcatcatattgctaatgatgctattcgtgattggatttttccagaatatgataaattaaaaaaagaaaatcgtttagattttgaaccatcaccatatgatgttgctttaattggtgattataatattggtggtgatgcttgggcttcacgtatgttattagaagaaatgggtttacgtgttgttgctcaatggtcaggtgatggtacattaaatgaattaattcaaggtccagctgctaaattagttttaattcattgttatcgttcaatgaattatatttgtcgttcattagaagaacaatatggtatgccatggatggaatttaatttttttggtccaacaaaaattgctgcttcattacgtgaaattgctgctaaatttgattcaaaaattcaagaaaatgctgaaaaagttattgctaaatatacaccagttatgaatgctgttttagataaatatcgtccacgtttagaaggtaatacagttatgttatatgttggtggtttacgtccacgtcatgttgttccagcttttgaagatttaggtattaaagttgttggtacaggttatgaatttgctcataatgatgattataaacgtacaacacattatattgataatgctacaattatttatgatgatgttacagcttatgaatttgaagaatttgttaaagctaaaaaaccagatttaattgcttcaggtattaaagaaaaatatgtttttcaaaaaatgggtttaccatttcgtcaaatgcattcatgggattattcaggtccatatcatggttatgatggttttgctatttttgctcgtgatatggatttagctttaaattcaccaacatggtcattaattggtgctccatggaaaaaagctgctgctaaagctaaagctgctgcttaactcgagagatacaacaatgccacaaaatccagaacgtacagttgatcatgttgatttatttaaacaaccagaatatacagaattatttgaaaataaacgtaaaaattttgaaggtgctcatccaccagaagaagttgaacgtgtttcagaatggacaaaatcatgggattatcgtgaaaaaaattttgctcgtgaagctttaacagttaatccagctaaaggttgtcaaccagttggtgctatgtttgctgctttaggttttgaaggtacattaccatttgttcaaggttcacaaggttgtgttgcttattttcgtacacatttatcacgtcattataaagaaccatgttcagctgtttcatcatcaatgacagaagatgctgctgtttttggtggtttaaataatatgattgaaggtatgcaagtttcatatcaattatataaaccaaaaatgattgctgtttgtacaacatgtatggctgaagttattggtgatgatttaggtgcttttattacaaattcaaaaaatgctggttcaattccacaagattttccagttccatttgctcatacaccatcatttgttggttcacatattacaggttatgataatatgatgaaaggtattttatcaaatttaacagaaggtaaaaaaaaagctacatcaaatggtaaaattaattttattccaggttttgatacatatgttggtaataatcgtgaattaaaacgtatgatgggtgttatgggtgttgattatacaattttatcagattcatcagattattttgattcaccaaatatgggtgaatatgaaatgtatccaggtggtacaaaattagaagatgctgctgattcaattaatgctaaagctacagttgctttacaagcttatacaacaccaaaaacacgtgaatatattaaaacacaatggaaacaagaaacacaagttttacgtccatttggtgttaaaggtacagatgaatttttaacagctgtttcagaattaacaggtaaagctattccagaagaattagaaattgaacgtggtcgtttagttgatgctattacagattcatatgcttggattcatggtaaaaaatttgctatttatggtgatccagatttaattatttcaattacatcatttttattagaaatgggtgctgaaccagttcatattttatgtaataatggtgatgatacatttaaaaaagaaatggaagctattttagctgcttcaccatttggtaaagaagctaaagtttggattcaaaaagatttatggcattttcgttcattattatttacagaaccagttgatttttttattggtaattcatatggtaaatatttatggcgtgatacatcaattccaatggttcgtattggttatccattatttgatcgtcatcatttacatcgttattcaacattaggttatcaaggtggtttaaatattttaaattgggttgttaatacattattagatgaaatggatcgttcaacaaatattacaggtaaaacagatatttcatttgatttaattcgttaggcggccgctcgctaccttaggaccgttatagttattaccctgttatccctaaccggtggaggcttcttgttatgacatgtttttttggtgttcaatcaatgccacgtgcttcaaaacaacaagctcgttatgctgttggtcgttgtttaatgttatggtcatcaaatgatgttacacaacaaggttcacgtccaaaaacaaaattaaatattatgggtgaagctgttattgctgaagtttcaacacaattatcagaagttgttggtgttattgaacgtcatttagaaccaacattattagctgttcatttatatggttcagctgttgatggtggtttaaaaccacattcagatattgatttattagttacagttacagttcgtttagatgaaacaacacgtcgtgctttaattaatgatttattagaaacatcagcttcaccaggtgaatcagaaattttacgtgctgttgaagttacaattgttgttcatgatgatattattccatggcgttatccagctaaacgtgaattacaatttggtgaatggcaacgtaatgatattttagctggtatttttgaaccagctacaattgatattgatttagctattttattaacaaaagctcgtgaacattcagttgctttagttggtccagctgctgaagaattatttgatccagttccagaacaagatttatttgaagctttaaatgaaacattaacattatggaattcaccaccagattgggctggtgatgaacgtaatgttgttttaacattatcacgtatttggtattcagctgttacaggtaaaattgctccaaaagatgttgctgctgattgggctatggaacgtttaccagctcaatatcaaccagttattttagaagctcgtcaagcttatttaggtcaagaagaagatcgtttagcttcacgtgctgatcaattagaagaatttgttcattatgttaaaggtgaaattacaaaagttgttggtaaataatctcgagctcaagcttcgaattctgcagtcgacggtaccgcgggcccgggatccacctgatctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacgcggccgcattaccctgttatccctattaattaagagctcgctaccttaagagaggatatcggcgcgcctttttttttaaactaaaataaatctggttaaccatacctggtttattttagtttatacacacttttcatatatatatacttaatagctaccataggcagttggcaggacgtccccttacgggacaaatgtatttattgttgcctgccaactgcctaatataaatattagtggacggatcccccgggctgcaggcatgcaagctaattcccgatctagtaacatagatgacaccgcgcgccgcggaagaatagaagttacaacgtaattctacgttgggttaatttattaaccccttaaaggggttgcatttagaatccaggcatcttgggtaaattatgtataataaaactatattaaaagttaattcattaaagccgtttatttaaaataaataaaaaattatgtcacgttatttaggccctcgtttgagagttattcgtcgtattggtaaattaagaggttttacgcgtaaaaaacctttccgtcgtgtatttaaaggttttggtggttttaaaggtaaagttattcctccaggtcaacatggtttaacaaaactattaaaaacaagaccatatgattcatctgaatcggattatctaatccgtttaaaagtaaaacaacgtttacgttttaactatggtattactgaacgtcaacttgttaactatgtacgtaaagctaaaaaaattaaagaatctacaggtcaagttttactacaatttttagaaatgcgtttagataatattgtattccgtttaaatatggcacctacaattccagcagcacgccaacttattagtcatggacatattcgtgtaaataataaaaaagtaaatattcctagctatatgtgtaaaccaaaagatgttatttctgtagcaatgaaacaacgttcgttacaacttgtaaataaaaacttacaagaatattaccgtcgtatgcgtttctataaaaaacgtttagaaaaaacattaccttttattttactaaaaattaaaccattaggtcttactagtgtaacagcagctgtagaacttattactaaaggaaacgtgcgagtaaataataagagtgtgaaaacgccaaattatatttgtcgtccacgagatacagtttctttaagaacaaaacaaggtattaaaaaagtatttttaaaaaattatttaaaaggttaatttaatcggtaaaattcccaggtagttgcacctttcccttcaggacgtccccttccccttcggacaataaataaatttgttgcctgccaacaaatttatttattgtattaaaatagaataaaatttatttgctgcggtagcaggtttacatacaatttatttattgtaccactccactggcgtccttcggagtatgtaaacatgcttggcactggtttacatacaatttatttattgtaccactgccactgcgtcctccttcggagtatgtaaacatgctaagtttacttgcccaatatttatattaggcagttggcaggcaactcactaaaatttatttacccgaaggacgtcccgaagggaaggggaaggaggacgtccccttacgggaatataaatattagtggacgtcagtggcggtaccactgccactggcgtcctatatttatatactcctaagtttacttgcccaatatttatatacccgaaggaaggggaagcatataaatatacttcgggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 4 - DNA, 547 bp, Chlamydomonas reinhardtii
gcctgccaactgcctatatttatatactgcgataaactttagtcccgaaggggtttacatatccgaaggaggaagcaggcagtggcggtaccacgccactggcgtcctaatataaatattgggcaagtaaacttagaataaaatttatttgctgcgttagcaggtttacatactcctaagtttacttgcccgaaggggaaggaggacgtcccctacgggaatataaatattagtggcagtggtacaataaataaattgtatgtaaaccccttcgggcaactaaagtttatcgcagtattaacatcctagtatataaatatcggcagttggcaggcaacaaatttatttattgtcccgtaaggggaaggggaaaacaattattattttactgcggagcagcttgttattagaaatttttattaaaaaaaaaataaaaatttgacaaaaaaaaataaaaaagttaaattaaaaacactgggaatgttctaacaatcataaaaaaatcaaaagggtttaaaatcccgacaaaatttaaactttaaagagt

SEQ ID NO: 5 - DNA, 32 bp, Artificial Sequence
  TR-F, Adapter primer (SacI), DNA Tomato
aagagctcgctgcagcaggatttgaaaaagga

SEQ ID NO: 6 - DNA, 34 bp, Artificial Sequence
  TR-R, Adapter primer (SacII), DNA Tomato
ttccgcggaactagtaattaattcccgcctttcg

SEQ ID NO: 7 - DNA, 33 bp, Artificial Sequence
  TL-F, Adapter primer (Xho1), DNA Tomato
aactcgagaccagaataagatgttgcggagaca

SEQ ID NO: 8 - DNA, 30 bp, Artificial Sequence
  TL-F, Adapter primer (Kpn1), DNA Tomato
aaaggtaccgattggggcgtggacataagg

SEQ ID NO: 9 - DNA, 34 bp, Artificial Sequence
  NifH atg Bgl11, Adapter primer (Bgl11), DNA Cyanobacteria
aaaagatctatgactgacgaaaacattagacaga

SEQ ID NO: 10 - DNA, 33 bp, Artificial Sequence
  NifH Stop Not1, Adapter primer (Not1), DNA Cyanobacteria
tttgcggccgctatttggtagcttctgcgggct

SEQ ID NO: 11 - DNA, 37 bp, Artificial Sequence
  NifD ATG, Adapter primer (Age1), DNA Cyanobacteria
aaaccggtggaggcagactatgacacctcctgaaaac

SEQ ID NO: 12 - DNA, 27 bp, Artificial Sequence
  NifD Stop, Adapter primer (XhoI), DNA Cyanobacteria
ttctcgagttaggcagcagccttagcc

SEQ ID NO: 13 - DNA, 34 bp, Artificial Sequence
  NifK ATG, Adapter primer (XhoI), DNA Cyanobacteria
aactcgaggagatacaacaatgcctcagaatcca

SEQ ID NO: 14 - DNA, 33 bp, Artificial Sequence
  NifK Stop, Adapter primer (NotI), DNA Cyanobacteria
aaaagcggccgctagcggatcaagtcaaaggag

SEQ ID NO: 15 - DNA, 31 bp, Artificial Sequence
  MCS F, Adapter primer (XhoI), DNA pPZP-RCS
aactcgagtccgcataaagaaccacccataa

SEQ ID NO: 16 - DNA, 33 bp, Artificial Sequence
  MCS R, Adapter primer (SacII), DNA pPZP-RCS
tttccgcggcgcgcggtgtcatctatgttacta

SEQ ID NO: 17 - DNA, 35 bp, Artificial Sequence
  TPsbA F, Adapter primer (BamHI), DNA Tomato
tttggatccatcatgaataaatgcaagcaaatacc

SEQ ID NO: 18 - DNA, 38 bp, Artificial Sequence
  TPsbA R, Adapter primer (AscI), DNA Tomato
tttggcgcgccgatcctagcctagtctataggaggttt

SEQ ID NO: 19 - DNA, 29 bp, Artificial Sequence
  Prrn165 R, Adapter Primer (BamHI) DNA Tomato)
ccaaccggtcgccgtcgttcaatgagaat

SEQ ID NO: 20 - DNA, 36 bp, Artificial Sequence
  Prrn16S R, Adapter primer (BamHI), DNA Tomato
tttggatccttcatagttgcattacttatagcttcc

SEQ ID NO: 21 - DNA, 33 bp, Artificial Sequence
  NifH-20 ATG, Adapter primer (BglII), DNA Cyanobacteria
aaaagatctaccaaccaattgcaggaaaagaga

SEQ ID NO: 22 - DNA, 38 bp, Artificial Sequence
  NifH-6 ATG, Adapter primer (BglII), DNA Cyanobacteria
aaaagatctaggaggagaacaatgactgacgaaaacat

SEQ ID NO: 23 - DNA, 35 bp, Artificial Sequence
  aada ATG, Adapter primer (AgeI), DNA pPZP-RCS
aaaccggtggaggcttcttgttatgacatgttttt

SEQ ID NO: 24 - DNA, 32 bp, Artificial Sequence
  aada STOP, Adapter primer (XhoI), DNA pPZP-RCS
aactcgagattatttgccgactaccttggtga

SEQ ID NO: 25 - DNA, 25 bp, Artificial Sequence
  Nif H, Adapter primer (N/A), DNA Cyanobacteria
atgactgacgaaaacattagacaga

SEQ ID NO: 26 - DNA, 30 bp, Artificial Sequence
  Derived from Chlorogloeopsos fritschii (NIF-H -K, #912, respectively)
ctcgagatgactgacgaaaacattagacag

SEQ ID NO: 27 - DNA, 31 bp, Artificial Sequence
  Nif-H XbaI, Adapter primer (XbaI), DNA Cyanobacteria
tctagaatgactgacgaaaacattagacaga

SEQ ID NO: 28 - DNA, 193 bp, Solanum lycopersicum
cgtcgttcaatgagaatggataagaggctcgtgggattgacgtgagggggcagggatgactatatttctgggagcgaactccgggcgaatatgaagcgcatggatacaagttatgccttggaatgaaagacaattccgaatccgctttgtctacgaacaaggaagctataagtaatgcaactatgaaggatct

SEQ ID NO: 29 - DNA, 984 bp, Artificial Sequence
  Derived from Cyanothece ATCC51142 (NIF-H; #42)
atgggccgcaacagccagggcttcctgacgacgatccgcatcatcgtcacgagcgcgacggaccagccgagcaacttcatccacagcctgagcaacaagcgcgagagcacgatgcgccagatcgcgttctacggcaagggcggcatcggcaagagcacgacgagccagaacacgatcgcggcgctggcggagacgaaccgcatcatgatcgtcggctgcgacccgaaggcggacagcacgcgcctgatgctgcacacgaaggcgcagacgacgatcctgcacctggcggcggagcgcggcacggtcgaggacatcgagctggaggaggtcctgctggagggctaccagggcgtcaagtgcgtcgagagcggcggcccggagccgggcgtcggctgcgcgggccgcggcatcatcacggcgatcaacttcctggaggaggagggcgcgtacgaggacctggacttcgtcagctacgacgtcctgggcgacgtcgtctgcggcggcttcgcgatgccgatccgcgagggcaaggcgcaggagatctacatcgtcacgagcggcgagatgatggcgatgtacgcggcgaacaacatcgcgcgcggcatcctgaagtacgcgcacacgggcggcgtccgcctgggcggcctgatctgcaacagccgcaacgtcaactgcgaggcggagctgatcgaggagctggcgcgccgcctgggcacgcagatgatccacttcgtcccgcgcagcaagcaggtccaggaggcggagctgcgccgcatgacggtcatcgagtacagcccggaccacccgcaggcgcaggagtaccgcgagctgagccgcaagatcgagaacaacacgaacctggtcatcccgacgccgatcacgatggaggagctggaggagctgctggtcgacttcggcatcctgggcggcgaggacgagtacgagaaggcgctgcaggcggacaaggcggcgacgaaggcgtag

SEQ ID NO: 30 - DNA, 1551 bp, Artificial Sequence
  Derived from Cyanothece ATCC51142 (NIF -D, #42)
atggttgcacctttcactggtctcaaatcttcagccactttccctgttacaaagaagcaaaaccttgacatcacttccattgctagcaatggtggaagagttagctggatggctactgtagaagataataaaaaattaattgctgatgtattatctacttatcctgaaaaagctgctaaaaaaagagctaaacatttaggagtatatgaagaaggagaagctgattgtggagtaaaatctaataaacaatctttacctggagtaatgactgctagaggatgtgcttatgctggatctaaaggagtagtatggggacctattaaagatatggtacatatttctcatggacctgtaggatgtggatattattcttggtctggaagaagaaattattatattggaactactggagtagattcttttggaactatgcaatttacttctgattttcaagaaagagatattgtatttggaggagataaaaaattagctaaaattattgatgaaattgaagaattatttcctttaaatggaggagtatctgtacaatctgaatgtcctgtaggattaattggagatgatattgaatctgtagctagaactaaatctaaagaaactggaaaatctgtagtacctgtaagatgtgaaggatttagaggagtatctcaatctttaggacatcatattgctaatgatatgattagagattgggtatttcctactgctgataaagaaaatgctgaaaaaggatttgaaggaactccttatgatgtagctattattggagattataatattggaggagatgcttggtcttctagaattttattagaagaaattggattaagagtagtagctcaatggtctggagatggaactttaactgaaatgaaagctactcctaatgtaaaattaaatttaattcattgttatagatctatgaattatatttctagacatatggaagaaaaatatggaattccttggttagaatataatttttttggaccttctaaaattgctgcttctttaagagaaattgcttctagatttgatgaaaaaattcaagctaaagctgaagaagtaattgaaaaatatagaaaacaatctgaagaaattattgctaaatatagacctagattagaaggaaaaactgtaatgatgatggtaggaggattaagacctagacatgtagtacctgcttttaaagatttaggaatggaaattattggaactggatatgaatttgctcatggagatgattataaaagaactactggatatgtagaagatgctactttaatttatgatgatgtaactggatatgaatttgaagaatttgtaaaagaattaaaacctgatttagtagctgctggaattaaagaaaaatatgtatttcaaaaaatggctttaccttttagacaaatgcattcttgggattattctggaccttatcatggatatgatggatttgctatttttgctagagatatggatttagctttaaattctcctacttggggattaattggaactccttggaataaataa

SEQ ID NO: 31 - DNA, 1536 bp, Artificial Sequence
  Derived from Cyanothece ATCC51142, (NIF-K, #42)
atggctcaaaatgtaaataatattaaagatcatgtagatttatttcatcaacctgaatatcaagaattatttgaaaataaaaaacaatttgaaggaatgcctactgctgaaaaagtacaagaagtagctgaatggactaaatcttgggaatatagagaaaaaaattttgctagagaagctttaactgtaaatcctgctaaagcttgtcaacctttaggagctttattagctgctattggatttgaaggaactttaccttttgtacatggatctcaaggatgtgtagcttattttagaactcatttaactagacattttaaagaacctgtatctgctgtatcttcttctatgactgaaaatgctgctgtatttggaggattaaaaaatatggtagatggattacaaaattcttatgctttatataaacctaaaatgattgctgtatgtactacttgtatggctgaagtaattggagatgatttaggagcttttttaggaaatgctagacaagatggagtaattcctgatgatttacctgtaccttttgctcatactccttcttttgtaggatctcatattactggatatgattctatgatgaaatctattttatctactttaactgaaggaaaaaaaaaagaaactactaatggaaaaattaattttattgctggatttgaaacttatattggaaatgtaagagctattaaaaatattatttctgcttttgatttagaaggaactttattatctgatactgaaatgtatttagattctcctaatttaggagaatttaaaatgtatcatgaaggaacttctttagaagatgctgctgattctattaatgctgaagctactgtaactttacaaacttatactactcctaaaactagagaatatattgaaaaaaaatggggacaaaaaacttatacttatagaccttggggagtaaaaggaactgatgaatttttaatgggattatctgaattaactggaaaacctattcctaaagaatttgaaattgctagaggaagagctgtagatgctatgactgatactcaagcttgggtacatggaaaaagagctgctgtatatggagatcctgatttagtaatgggattattacaatttatgttagaaatgggaattgaacctgtacatgtattagtaaataattctactaaagaatttgaagaagaagctaaagctttattagctgcttctccttatggacaacaagctactgtatggggaggaaaagatttatggcatatgagatctttattatttactgaacctgtagattttttagtaggaaattcttatgctaaatatttacaaagagatactaaaactcctttaattagaattggatatcctatttttgatagacatcatttacatagatattctactattggatatgaaggagctattaatttattaaattggattgctaatggattaatggatgaattagatagaaaaactgatactccttctgtaactgatatttcttttgatttagtaagatag

SEQ ID NO: 32 - DNA, 891 bp, Artificial Sequence
  Derived from Trichodesmum sp. IMS101 (NIF-H, #101)
atgagacaaattgctttttatggaaaaggaggaattggaaaatctactacttctcaaaatactttagctgctatggctaatagacatggacaaagaattatgattgtaggatgtgatcctaaagctgattctactagattaattttaaatgctaaagctcaaactactgtattacatgtagctgctgaaagaggagctgtagaagatgtagaattagatgaagtattaaaacctggatttggaggaattaaatgtgtagaatctggaggacctgaacctggagtaggatgtgctggaagaggaattattactgctattaattttttagaagaagaaggagcttatactgatttagattttgtatcttatgatgtattaggagatgtagtatgtggaggatttgctatgcctattagagaaaataaagctcaagaaatttatattgtatgttctggagaaatgatggctatgtatgctgctaataatattgctagaggagtattaaaatatgctcatgctggaggagtaagattaggaggattaatttgtaattctagaaaagtagatagagaaactgaattaattgaaaatttagctgctagattaaatactcaaatgattcattttgtacctagagataatgtagtacaaagagctgaaattagaagaatgactgtagaacaatatgctcctgaagataatcaagctcaagaatatgatcaattagctcaaaaaattattaataatgaaaaattaactattcctactcctttagaaatggatgaattagaagaattattaattgaatttggattattaggagatgaagaagatagacaaaaacaaattgctgctcaagatgctgctattaaagctactgctgctaaataa

SEQ ID NO: 33 - DNA, 1458 bp, Artificial Sequence
  Derived from Trichodesmum sp. IMS101 (NIF-D #101)
atggcttctgaaaaaattgaacaaaataaacaattaattcaagaagtattagatgcttatcctgctaaagctgctaaaagaagaaaaaaacatttaaatgtaattgaagaaaaaggagctgattgtggagtaaaatctaatgtaaaatctgtacctggagtaatgactactagaggatgtgcttttgctggagctaaaggagtagtatggggacctgtaaaagatatggtacatatttctcatggacctgtaggatgtggatattattcttgggctggaagaagaaattattataatggagtaactggagtagatacttttggaactatgcaatttacttctgattttcaagaaagagatattgtatttggaggagataaaaaattagctaaaattatgaatgaaattgaagaattatttcctttaaatgctggaattactattgaatctgaatgtcctgtaggattaattggagatgatattgaagctgtagctaaaaaagcttctaaagaattaaataaacctgtagtacctgtaagatgtgaaggatttagaggagtatctcaatctttaggacatcatattgctaatgatactgtaagagattgggtatatgaaccttctgctaaagtaactaatgaagaaattggatttgaaaaaactccttatgatgtatctttaattgctgattataatattggaggagatggatggtcttctagattattattagatgaaattggattaagagtagtatctcaagctactggagatggaacttataatgaagtatttatggctcctagagtaaatttaaatttaattcattgttatagatctatgaattatatttgtagatatatggaagaagaatatggaattccttgggtagaatttaatttttttggaccttctcaaattgctaaatctttaagaaaaattgcttctttttttgatgataaaattaaagaaaatactgaaaaagtaattgctagatatcaagaacaagctgatgctgtaattgctaaatatagacctagattagaaggaaaaaaagtaatgatgatggtaggaggattaagacctagacatattattcctgcttttgatgatttaggaatggaagtaattggaactggatatgaatttggacatggagatgattataaaagaactgctgattatgctcaagaaggaactttaatttatgatgatgtatctggatatgaatttgaagaatttgctaaaaaattaaaacctgatttaattgcttctggaattaaagaaaaatatgtatttcaaaaaatgggaatgccttttagacaaatgcattcttgggattattctggaccttatcatggatatgatggatttgctatttttgctagagatatggatttagctttaaattctcctacttggaatttaattaaagctccttggaaacaagctaaatag

SEQ ID NO: 34 - DNA, 1539 bp, Artificial Sequence
  Derived from Trichodesmum sp. IMS101 (NIF-K, #101)
atgtctcaaaatgtagataaaattaaagatcattttcaattatttcaagaacctgaatatcaagaaatgtttgctagaaaaagagaatttgaaggaggagcttctaaagaagaaattgaaagagtaagagaatggactaaatcttgggaatatagagaaaaaaattttgctagagaagctttaactattaatcctgctaaagcttgtcaacctttaggagctatttttgctgctgctggatttgaaggaactttaccttttgtacatggatctcaaggatgtgtagcttattttagatctcatttaactagaaattataaagaaccttttcaagctgtatcttcttctatgactgaagatgctgctgtatttggaggattaaaaaatatgattgatggattagctaattcttatgctttatataaacctaaaatgattgctttatgtactacttgtatggctgaagtaattggagatgatttaggatcttttattactaattctaaaaatgaaggagctgtacctcaagattttcctgtaccttttgctcatactccttcttttgtaggatctcatattactggatatgataatatgttaaaaggaattttaattgctttaactgatggaaaaaaaactgaaactgataatggaaaaattaattttattcctggatttgatccttatattggaaatattagagatttaaaaaatattttatctttaatggatgtaccttctactgtattagctgataatgctgaatcttttgattctcctaatttaggagaatttaaaatgtataatggaggaactactttagaagaagctggagattctattaatgctaaagctactatttcttttcaaaaatattctactcctaaaactttagaatatttaaaacaagaaggaggacaaaaaactgctacttatagacctattggagtaagaggaactgatgaatttttaatggctttatctgaattaactggaaaagctattcctgaagaattagaaattgaaagaggaagagtagtagatgctattactgattctcaagcttggttacatggaaaaagaattgctatttatggagatcctgatcatgtattaggattattaaattttactttagaattaggaatgcaacctgtacatgtagtagtaaataatggaaatgtagctggatttgaagaagaagctaaagaattattagctaatgatcctaatggaaaagaagctactgtatggattggaaaagatttatggcatttaagatctttattagatactgaacctgtagatttattaattggaaattcttatggaaaatttttacaaagagatactggaactcctttagtaagaattggatatcctatttttgatagacatcatcaacatagatattctattttaggatataaaggagcttttaatttaattaattggattgtaaatactattttagatgaattagatagaggatctatggatttaggagtaaatgatacttcttttgatttagtaagataa

SEQ ID NO: 35 - DNA, 894 bp, Artificial Sequence
  Derived from Chlorogloeopsos fritschii (NIF-H #912)
atgtctatcgataagaaaatccgtcaaatcgcattttacggaaaaggcggtatcggaaaatccactacctcacaaaacactcttgctgcaatggctgaaatgggtcaaagaatccttatcgttggatgcgatcctaaagcagacagtacccgccttatgcttcactctaaagctcaaaccaccgtacttcatcttgcagctgaacgtggtgctgttgaagatcttgaattagaggaagtaatgttaactggttttagaggagttaaatgcgtagagtctggtggtccagaaccaggtgttggatgtgcaggtcgaggaatcatcactgctatcaatttcttagaggaaaacggtgcatatcaagatgtagattttgtttcatacgatgttttaggtgatgtagtttgtggtggattcgctatgccgatccgtgagaacaaagcacaagaaatctatatcgtaaccagtggtgagatgatggctatgtacgcagctaataacatcgctagaggtatcttaaagtatgcacatactggaggggttcgattaggcgggttaatctgtaattcacgtaacgtagatcgtgaaatcgagttgatcgaaactttggctaaaagattgaatactcaaatgatccattacgttcctcgcgataacatcgtacaacatgccgagttgcgtcgcatgacagtaaatgaatatgcccctgattccaatcagtctaatgagtatcgtatcttggcgaataaaatcatcaataatgaaaatttgaaagtacctactcctatcgagatggaagaactagaagaactactgatcgaatttggtatcctggaatctgaagaaaatgctgctaaaatgatcgggactcctgctcagtcttccactaaataa

SEQ ID NO: 36 - DNA, 1494 bp, Artificial Sequence
  Derived from Chlorogloeopsos fritschii (NIF-D, #912)
atgactcctccagaaaatcaaaacctagtagatgagaataaagagttaatcaaagaagtgcttcaagcttaccctgaaaaggcacgaaaaaaaagagagaagcatttaaatgttcacgaagaaagtaaatccgattgtggtgtaaaatctaacataaagtccattccaggcgttatgactgctcgtgggtgcgcatatgctggtagcaaaggggtagtttggggtcctatcaaagatatgattcatatatcccacgggcctgtaggttgtgggtactggtcttggtccggaagacgtaattattacgttggtatcaccggaattaactctttcggtactatgcattttacttccgatttccaggaaagagacatcgtatttggaggtgataagaaattgaccaaaataattgatgagttagaaatcctttttccattaaatcgtggagtgtctattcaatcagaatgccctatcggtttgattggaggtgatatcgaagcagttgctaaaaaagcatctaagcagtatggaaaaccagtagttcctttaagatgtgagggtctgcgtggagtatcacaatctttaggtcatcacattgctaacgacgcaatcagagattggattttcccagaatttgataaggctaaaaaaaataatactatcgactttgaaccttcaccgtacgatgttgcacttattggagatcataacatcggtggagatgcttgggcatctcgtatgttattggaggaaatgggtctgagagtagttgctcaatggtcaggagatggtactttaaatgaacttattcaaggacctgcagctaaattggtactgattcactgctatcgttctatgaattacatctgtcgctcattggaggaacaatatggtatgccgtggatggaattcaacttttttggacctactaaaattgcagcttctcttcgtgagatcgcagctaaattcgacagtaaaattcaagaaaatgcagaaaaagttatcgctaaatacaccccggtaatgaatgcagtgcttgagaaatatcgccctcgtttggaaggtaacactgttatgctttacgtagggggtcttcgccctcgtcatgttgtacctgctttcgaagatttggggattaaagttgtaggtaccgggtatgaatttgcacataatgatgattacaaacgcactacacactatatcgataacgctactattatctacgatgatgttacagcctatgagttcgaagaatttgtaaaagctaaaaaacctgatcttattgcctctggtatcaaagaaaaatacgcttttcagaaaatgggtctaccttttcgacaaatgcatagttgggattattctggtccttatcatggttatgatcgttttgccatctttgctcgagatatggatcttgccctaaatagtcctacttggagtctaatcggtgcgccttggaaaaaagcggctactaaagcgaaagctgctgcttaa

SEQ ID NO: 37 - DNA, 1539 bp, Artificial Sequence
  Derived from Chlorogloeopsos fritschii (NIF-K, #912)
atgcctcaaaatccagagaaaattgtagatcatgtacaattatttcagcaacctgaatatcaagaacttttcaaaaataaacacgaacaatttgagggagctcattccccagaagaagtacagagagtatccgaatggactaaaggttgggagtacagagaaaagaactttgcacgtcaagctttaacagtaaatcctgcaaaaggatgccaaccagtaggtgctatcttcgcagctgtaggatttgaaggtactctacctttcgtacaaggatcccagggttgtgtagcatattttagaacacatttatcccgtcactacaaagaaccattctctgctgtatcctcatctatgactgaggatgcagctgtatttggaggtcttaacaatatgattgaaggattacaagtatcatatcaactatacaaacctaaaatgatcgcagtatgtacaacttgtatggctgaagtaattggtgatgatttaggagcattcatcacaaatgctaaaaacgcaggttctattccacaagattttcctgtaccattcgctcatactccttcatttgtaggatctcatacaaccggttatgataatatgatgaagggaatcctttcaaatttaactgagggtaaaaaaaaagataagaccaacggaaaaattaatttcatcccaggttttgatacttacgtaggaaataacagagaactgaaacgtatgttaggtcttatgggaattgattataccatcttagcagataattctgattacctggatgctcctaatactggtgaatatgatatgtacccgggcggtaccaaattagaggatgcagctgattcatgcaacgcagaagctactgtagcccttcagagatataccactaccaagactcgagaatacattgagaccaaatggaaacaacaaactaaagtattacgtcctttcggggtaaaaggtaccgacgaatttcttatgactttatctgaaatgaccgggaaaccgatccctcaagaacttgaggatgaacgaggtcgtttagtagacgctatgactgatagttatgcctggattcacgggaaaaaattcgctatctacggtgatccggaccttatttattctgtaaccagttttttacttgaattaggggccgaacctgtacatatcctttgtaataatggtgatgaagagttcaaaaaagagatggaagctattttatctgcgagtccgtttgggaaacaagctactgtatggatccagaaagatttgtggcatcttcgatctttgctttttactgaacctgtagatttttttgtaggtaacagttacgggaaatatttgtggcgcgacaccaaaattcctatggtacgtatcggttatcctctttttgatcgccaccatttgcatcgttatcctactttggggtatcaaggtggtttgaatttgcttaattgggtagtaaacactattttggatgaacttgatcgcagtagcaatatagcgggtaaaactgatatatcttttgatttgatccgttaa

SEQ ID NO: 38 - DNA, 1206 bp, Artificial Sequence
  Derived from Anabaena. (NIF-H, #AB)
atggcttcctctgtcatttcttcagcagctgttgccacacgcagcaatgttacacaagctagcatggttgcacctttcactggtctcaaatcttcagccactttccctgttacaaagaagcaaaaccttgacatcacttccattgctagcaatggtggaagagttagctgcgagctcaagcttcgaattctgcagtcgacggtaccgggccccccctcgaggtcgacaatgactgacgaaaacattagacagatagctttctacggtaaaggcggtatcggtaaatctaccacctcccaaaacacccttgcagctatggcagaaatgggtcaacgcatcatgatcgtaggttgcgaccctaaagctgactccacccgtttgatgctccacgctaaagcgcaaaccactgtacttcacttggctgctgaacgcggtgcagtagaagacttagaactacacgaagtaatgttgaccggtttccgtggcgttcgttgcgtagaatctggtggtccagaacccggtgtaggttgcgccggtcgtggtatcatcaccgccattaacttcttagaagaaaacggtgcttaccaagacctagacttcgtatcctacgacgtattgggtgacgttgtatgtggtggtttcgctatgcctatccgtgaaggtaaagcacaagaaatctacatcgttacctctggtgaaatgatggcgatgtatgctgctaacaacatcgctcgcggtattttgaaatatgctcactccggtggtgtacgcttgggtggtttgatttgtaacagccgtaaaactgaccgggaagccgaattgatcgaaaacttggctgaacgtttgaacactcaaatgattcacttcgtacctcgtgacaacatcgttcaacacgctgaattgcgtcggatgactgttaacgagtacgcaccagacagcaaccaaggtcaagagtaccgtgcattagccaagaaaatcatcaacaacgacaagctcaccattcctacaccaattgaaatggatgaactcgaagccctgttgatcgaatacggtattcttgatgatgattctaagcacgcagaaatcatcggtaagcccgcagaagctaccaaattggatccacctgatctagagtccgcaaaaatcaccagtctctctctacaaatctatctctctctatttttctccagaataatgtgtgagtag

SEQ ID NO: 39 - DNA, 1494 bp, Artificial Sequence
  Derived from Anabaena. (NIF-D #AB)
atgacacctcctgaaaacaagaatcttgtagatgaaaataaagaacttattaaagaagttctgaaagcttatcccgaaaaatctcgcaaaaaacgcgaaaagcacctcaacgtccacgaagaaaacaagtctgattgcggcgtaaagtctaacatcaaatccgttcctggtgtaatgaccgctcgtggttgtgcttatgcaggttctaagggtgtggtttggggtcctattaaggacatgattcacatcagccacgggcctgtaggttgcggttactggtcttggtctggtcgtcgtaactactacgttggtgtaactggtatcaactctttcggtaccatgcactttacatcagacttccaagaacgtgacatcgtgttcggtggtgacaaaaaactcgttaaactcattgaagaacttgacgttctgttccctctaaaccgtggtgtttccattcaatctgaatgtcccatcggtctaattggggatgacatcgaagctgtagctaagaaaacttctaagcaaattggtaagcctgttgtacccttacgttgcgaaggtttccgtggtgtatctcagtctttaggacaccacatcgctaacgacgctatccgtgactggattttcccagaatacgacaagctgaagaaagaaaacagactcgacttcgagccaagcccctatgatgtagccctaatcggtgactacaacatcggtggtgacgcttgggctagccgtatgttgttggaagaaatgggcttacgtgttgtagctcagtggtctggtgatggtactcttaacgagttgatccaaggccctgctgctaagttagtcctcatccactgctaccgttctatgaactacatctgccgtagtttggaagaacaatatggtatgccttggatggagttcaacttcttcggccccaccaagattgctgcttccttacgtgaaatcgcagctaagtttgattccaagattcaagaaaacgctgagaaggtaattgctaagtacacaccagtaatgaatgctgtacttgataagtaccgtcctcgtttggaaggtaacaccgtaatgttgtacgtaggtggtctacgtcctcgtcacgttgttcctgcttttgaagatttgggtatcaaagttatcggtacaggttacgagttcgcccacaacgacgactacaaacgtaccacccactacatcgataacgccaccatcatttatgatgacgttaccgcctacgaatttgaagagttcgtaaaagctaagaagcctgatttaatcgcttctggtattaaagagaagtatgtcttccaaaagatggctcttcccttccgtcaaatgcactcttgggattactccggcccttatcacggttacgacggatttgctatcttcgcccgtgacatggatttagcgctcaacagccccacttgggacttgattggcgctccttggaagaaagctgctacaaaagctaaggctgctgcctaa

SEQ ID NO: 40 - DNA, 1539 bp, Artificial Sequence
  Derived from Anabaena.  (NIF -K, #AB)
atgcctcagaatccagaaagaattgtagaccacgttgatctattcaaacagccagaatacaccgagctatttgaaaacaagagaaagaactttgaaggcgctcatcctcctgaagaagttgaaagagtgtctgaatggacaaaatcttgggactaccgcgaaaagaacttcgctcgtgaagctttaaccgttaaccctgctaaaggttgccaacctgtaggcgcgatgttcgctgctttgggttttgaaggtactctacctttcgtacaaggttctcaaggttgcgttgcttacttccgtacacacctcagccgtcactacaaagagccttgctccgcagtatcttcttccatgacagaagacgcagcagtattcggtggcttgaacaacatggttgaaggtatgcaggttgcttaccaactgtacaagcctaagatgattgctgtttgcaccacctgtatggctgaggttatcggtgatgacttaggtgcgttcatcaccaactccaagaatgctggttctattcctcaagatttccctgtaccctttgctcacacccccagtttcgttggttctcacgtaactggttacgacaacatgatgaagggtattctgtctaacttgacagaaggtaagaagaaagctaccagcaacggtaaaatcaacatcatccctggttttgatacctatgtaggtaacaaccgcgaagttaagcggatgttgggtgtaatgggtgttgattacacaatcctgtctgacagcagcgactactttgattcaccaaacactggtgagtacgaaatgtacccaggtggtaccaagttggaagatgcggctgattctatcaacgctaaagctactgttgctctccaagcttacaccacacccaagacccgtgagtatatcaaaacccagtggaagcaagaaacacaagtattgcgcccctttggtgttaagggtactgacgagttcttaacagctatctctgaattgaccggtaaggctattcctgaagagttggaaatcgaacgcggtcgtttagttgatgctatcaccgactcttacgcttggattcatggtaagaagttcgctatttacggcgatccagatttgatcatctccatcaccagcttcttgttagagatgggtgctgaaccagtacacatcctctgcaacaacggtgatgacaccttcaaaaaagaaatggaagctatcctcgctgctagcccatttggtaaagaagctaaagtctggattcaaaaagacttgtggcacttccgttccttgttgttcaccgagcctgtagacttcttcatcggtaactcctacggtaagtacctgtggcgcgataccaagatcccaatggtgcggattggttatcctctcttcgatcgccaccacttacaccgttattctactctcggctaccaaggtggtctaaatatcctcaactgggttgttaacaccctgttggatgaaatggatcgcagcaccaacatcactggtaagaccgatatctcctttgacttgatccgctaa

SEQ ID NO: 41 - DNA, 255 bp, Solanum lycopersicum
atggctagcgctaccggactcagatctatggcttcctctgtcatttcttcagcagctgttgccacacgcagcaatgttacacaagctagcatggttgcacctttcactggtctcaaatcttcagccactttccctgttacaaagaagcaaaaccttgacatcacttccattgctagcaatggtggaagagttagctggagctcaagcttcgaattctgcagtcgacggtaccgcgggcccgggatccacctgatc

SEQ ID NO: 42 - DNA, 1044 bp, Arabidopsis thaliana
gagtctctgtcagaagtcagaactatctctctctcttgacttgtctaggtgactcttatagagctgtgaatttaaatggagttggattggtgtgatggtgaagataaattcagtgtcaattttggatggctggtgcagttttttttttccctcttaacgaaaacaaaattgaatttgttaaccaaataaagtaatggaatcttcataagtcaaactcatttttgatgtagttaaccaaataatgtcttcactttctttctcaatcaattattaacttcttgtgagaaataaactttcctaataatttgttcacgtataaatgtttttttaggttgaaaatttgaagtacaattttctcttattagtttttgttttcaagatatatctttgaattaatattatataaaaacgcagcatgtagtagtataacattttgatcttgaaaataaaaaaatatcaatttcaaagtcttgaaaataaccttttttttttttggctatgagtcttgtgaagttaacttggtgaactctcgactcacgatgcatatcttaattgagcaacatgattcataattcatcttttcatgtctcttgccaacttccaatagagtgcattgacattatttttttaatacattatacatatatcatgcatgtttgtttctcatccatggagaatcatttttgaaatttttggatttttgaaattgcttaaaaagaaagaaaaaaactcaatttttattgtcatacaatgttttccaaaatatgtgtacatgttcttgcatatgagttaattagtgagggtttcctcaaaaagattgttgtatttttggaaaacaaagtttatttttggatattggttctgcattttacatctaatttgttttttaaatcataattttagaaaatctattactaaattaagaaatctatgaatggtttacttcattccgtaaaacactataaattgtcctaaactcatattcgctctcaagtcaaaactcttctattttcccctgtcttcatatccaacagagatctcttc

SEQ ID NO: 43 - DNA, 1386 bp, Solanum lycopersicum
atatctttttgtcatgatataatatgtttcttttagcaaaaatgaaatagttccgtcatttatcggatcatatttagatagattgttgtagatgacacttttatgaaattacaataatcataaatttatcacgggcacaattagcgacaatgaagaccaaccaaagttagggggcaaaaaccgaacaggccaaccaaagttatgggtgtgcaaaaaatcgaatcgatcgataaatcgaatcgaaaaaatgttattgggttatttatgttttttcgtgggtttataaaaaaaattattgaattattggttcggtttcaatttttattattgggttattgggtaaaccgataatccaataagacggtaataatttttattttacccttcataattatttattattagcaagttaatatataattagacactataattatatcaaattattagtactctaccaacttcagagttggctgatttactagtttttattgtttattcaaaacctaagaattaaagtaatgcgttcacaattgcagttatttgattttaattttagttttagtcttattggactgttttattttagttttaggttataatggcaggttatagcatttgcatcttagtaaaggtcagtaatttgattaacacaaaaaattccatactatattttggtggtaatatgtaattatagcattcgtgctattttctcgtattgatacaatttctcattatctttcttgttttactatatcaaaacatttagagaagtgagaagacatataatattttacggacattttcttattgggtaaactgaaaatcgaatcgataatgataaaaaactgataaattgaaatctgataaaaaatatcttgttgatttgttattgaattaacatatttaaaagctgaaaaccgataaatcgaacgataatatataaaatcgaaccgaaccgatcgatgcacacccttgaccaaatctgaagcacatatttatcgatctaaattttattaaagagattaatatcgaataatcatatacatatttcatatgtataacaaatttcaaatacacgtatctaatatatcgagtgatgcgacaaatacatgtatcggacgcaccaattgatatagaaaacgtaatattgaaaactaatgtaaagaaaagtaacttgatcctaaactaatcaagataagcccaataaatatacattgtcatctccaaaggcccaaaaatggcacaagatggcaggcccaataacgaagaaaagggcttgtaaaaccctaataaagtggcactggcagagcttacactttcattccatcaacaaaagaaaccctagaagccgcagtgccactgatttctctcctccagacgaag

SEQ ID NO: 44 - DNA, 333 bp, Cauliflower mosaic virus
aaacatgtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatg

SEQ ID NO: 45 - DNA, 1374 bp, Solanum lycopersicum
aatattaggtggtgatcacgataggatacttgaaaaaaaatattgcaagtagacaaaccttgtagtaattgagacaaaatatcagtagctgtcaacaccacaaatatcataaatacctcactggctgtggggtttgtgctcgtgataaaacttatatattagtaataaaaaagaaacatagccacgtgaatgaaataatggaggtgaaaacagtaatgaaatgacaataattgagttcttgatccaaatgattgtttaagtttccaaattgtttctagtaactcaacgcaaaatattcttcaacactttttgacaaaatttgattggatctaaactagaactagtttgtaggctttgtgtgttaagtgattacaataaggttaataaatagagtaattcaaatcaaagcaaaaacagaaaaagatcaagttgagtagagaacttgatcacgatttaaacattttactatatatatgaggagctacattaaggataataagacaatttcatgctcaacatggttataattcaaactgttcaaagcaccatccagtaataggtcctacagtgtgtgatttaattttaattaaagttttatacatcaaatatagacgaaaaaaataaaaaataaatcttatttttgataatatatataaatttgaatgcattatgtttaataactatctatttcatctcgtgttgtttgtccttttaaagattcttttttatatttaatgactaagctaaatgtatagtaaatgcgttgaatttgactgaactcaaacaaaaaaacataggaatattacatttcaacgcaataactcaaatggtcaatgcattcacacgctaaatcaacatttttatatatacgcattaccacttatcaatctattaagatttattcatctccttaaaaaaagacacatttaattcctttaattataaaggtatttatcaaaaaacacccaaaacattacatatatctttctcataacatcttatatacatttactaaaaataataactagtaactaatttcttttctaaaaatacacattttttttgaaagaaaagaaaatacttatttgagatgataaattaatttattgggcgttaagaagagaacaaaaagaaaataatggatgccaaaaatatcggtggtgaaaatcgggggacccatattcttctcagcctataatgtagggtccatcagttattgcgtgtttatgtattcccgcctctctcctcattaccactagaaatatcactctctttcccatttctcccttcatcttcatcttcatcttcatctcttcttcttcttattctctcattcctctcatcaatttttttcatcaaaaaaactaagagaaa

SEQ ID NO: 46 - DNA, 16890 bp, Artificial Sequence
  Plasmid DNA host by E.Coli Xl1B and or Agrobacteriun EHA105-transformed into plant cell
tgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatccaagctcaagctaagcttcagagccaccaccctcagagccgccaccagaaccaccaccagagccgccgccagcattgacaggaggcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggatcatccgggtctgtggcgggaactccacgaaaatatccgaacgcagcaagatatcgcggtccatctcggtcttgcctgggcagtcgccgccgacgccgttgatgtggacgccgggcccgatcatattgtcgctcaggatcgtggcgttgtgcttgtcggccgttgctgtcgtaatgatatcggcaccttcgaccgcctgttccgcagagatcccgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcaggtaccgcgatcgctcgcgacctgcaggcataaagccgtcagtgtccgcataaagaaccaacccataatacccataatagctgtttgccaaccggtcaacatgtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacgtcgagagttctcaacacaacatatacaaaacaaacgaatctcaagcaatcaagcattctacttctattgcagcaatttaaatcatttcttttaaagcaaaagcaattttctgaaaattttcaccatttacgaacgatagccatggctagcgctaccggactcagatctatggcttcctctgtcatttcttcagcagctgttgccacacgcagcaatgttacacaagctagcatggttgcacctttcactggtctcaaatcttcagccactttccctgttacaaagaagcaaaaccttgacatcacttccattgctagcaatggtggaagagttagctgcagagctcaaatgggccgcaacagccagggcttcctgacgacgatccgcatcatcgtcacgagcgcgacggaccagccgagcaacttcatccacagcctgagcaacaagcgcgagagcacgatgcgccagatcgcgttctacggcaagggcggcatcggcaagagcacgacgagccagaacacgatcgcggcgctggcggagacgaaccgcatcatgatcgtcggctgcgacccgaaggcggacagcacgcgcctgatgctgcacacgaaggcgcagacgacgatcctgcacctggcggcggagcgcggcacggtcgaggacatcgagctggaggaggtcctgctggagggctaccagggcgtcaagtgcgtcgagagcggcggcccggagccgggcgtcggctgcgcgggccgcggcatcatcacggcgatcaacttcctggaggaggagggcgcgtacgaggacctggacttcgtcagctacgacgtcctgggcgacgtcgtctgcggcggcttcgcgatgccgatccgcgagggcaaggcgcaggagatctacatcgtcacgagcggcgagatgatggcgatgtacgcggcgaacaacatcgcgcgcggcatcctgaagtacgcgcacacgggcggcgtccgcctgggcggcctgatctgcaacagccgcaacgtcaactgcgaggcggagctgatcgaggagctggcgcgccgcctgggcacgcagatgatccacttcgtcccgcgcagcaagcaggtccaggaggcggagctgcgccgcatgacggtcatcgagtacagcccggaccacccgcaggcgcaggagtaccgcgagctgagccgcaagatcgagaacaacacgaacctggtcatcccgacgccgatcacgatggaggagctggaggagctgctggtcgacttcggcatcctgggcggcgaggacgagtacgagaaggcgctgcaggcggacaaggcggcgacgaaggcgtagtaggatccacctgatctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacgcggccgcacccataatacccataatagctgtttgccatcgctaccttaggaccgttatagttaaccggtcaacatgtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacgtcgagagttctcaacacaacatatacaaaacaaacgaatctcaagcaatcaagcattctacttctattgcagcaatttaaatcatttcttttaaagcaaaagcaattttctgaaaattttcaccatttacgaacgatagccatggctagcgctaccggactcagatctatggcttcctctgtcatttcttcagcagctgttgccacacgcagcaatgttacacaagctagcatggttgcacctttcactggtctcaaatcttcagccactttccctgttacaaagaagcaaaaccttgacatcacttccattgctagcaatggtggaagagttagctgcagagctcaaatggttgcacctttcactggtctcaaatcttcagccactttccctgttacaaagaagcaaaaccttgacatcacttccattgctagcaatggtggaagagttagctggatggctactgtagaagataataaaaaattaattgctgatgtattatctacttatcctgaaaaagctgctaaaaaaagagctaaacatttaggagtatatgaagaaggagaagctgattgtggagtaaaatctaataaacaatctttacctggagtaatgactgctagaggatgtgcttatgctggatctaaaggagtagtatggggacctattaaagatatggtacatatttctcatggacctgtaggatgtggatattattcttggtctggaagaagaaattattatattggaactactggagtagattcttttggaactatgcaatttacttctgattttcaagaaagagatattgtatttggaggagataaaaaattagctaaaattattgatgaaattgaagaattatttcctttaaatggaggagtatctgtacaatctgaatgtcctgtaggattaattggagatgatattgaatctgtagctagaactaaatctaaagaaactggaaaatctgtagtacctgtaagatgtgaaggatttagaggagtatctcaatctttaggacatcatattgctaatgatatgattagagattgggtatttcctactgctgataaagaaaatgctgaaaaaggatttgaaggaactccttatgatgtagctattattggagattataatattggaggagatgcttggtcttctagaattttattagaagaaattggattaagagtagtagctcaatggtctggagatggaactttaactgaaatgaaagctactcctaatgtaaaattaaatttaattcattgttatagatctatgaattatatttctagacatatggaagaaaaatatggaattccttggttagaatataatttttttggaccttctaaaattgctgcttctttaagagaaattgcttctagatttgatgaaaaaattcaagctaaagctgaagaagtaattgaaaaatatagaaaacaatctgaagaaattattgctaaatatagacctagattagaaggaaaaactgtaatgatgatggtaggaggattaagacctagacatgtagtacctgcttttaaagatttaggaatggaaattattggaactggatatgaatttgctcatggagatgattataaaagaactactggatatgtagaagatgctactttaatttatgatgatgtaactggatatgaatttgaagaatttgtaaaagaattaaaacctgatttagtagctgctggaattaaagaaaaatatgtatttcaaaaaatggctttaccttttagacaaatgcattcttgggattattctggaccttatcatggatatgatggatttgctatttttgctagagatatggatttagctttaaattctcctacttggggattaattggaactccttggaataaataagtaggatccacctgatctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacgcggccgcattaccctgttatccctaaccggtcaacatgtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacgtcgagagttctcaacacaacatatacaaaacaaacgaatctcaagcaatcaagcattctacttctattgcagcaatttaaatcatttcttttaaagcaaaagcaattttctgaaaattttcaccatttacgaacgatagccatggctagcgctaccggactcagatctatggcttcctctgtcatttcttcagcagctgttgccacacgcagcaatgttacacaagctagcatggttgcacctttcactggtctcaaatcttcagccactttccctgttacaaagaagcaaaaccttgacatcacttccattgctagcaatggtggaagagttagctgcagagctcaaatggctcaaaatgtaaataatattaaagatcatgtagatttatttcatcaacctgaatatcaagaattatttgaaaataaaaaacaatttgaaggaatgcctactgctgaaaaagtacaagaagtagctgaatggactaaatcttgggaatatagagaaaaaaattttgctagagaagctttaactgtaaatcctgctaaagcttgtcaacctttaggagctttattagctgctattggatttgaaggaactttaccttttgtacatggatctcaaggatgtgtagcttattttagaactcatttaactagacattttaaagaacctgtatctgctgtatcttcttctatgactgaaaatgctgctgtatttggaggattaaaaaatatggtagatggattacaaaattcttatgctttatataaacctaaaatgattgctgtatgtactacttgtatggctgaagtaattggagatgatttaggagcttttttaggaaatgctagacaagatggagtaattcctgatgatttacctgtaccttttgctcatactccttcttttgtaggatctcatattactggatatgattctatgatgaaatctattttatctactttaactgaaggaaaaaaaaaagaaactactaatggaaaaattaattttattgctggatttgaaacttatattggaaatgtaagagctattaaaaatattatttctgcttttgatttagaaggaactttattatctgatactgaaatgtatttagattctcctaatttaggagaatttaaaatgtatcatgaaggaacttctttagaagatgctgctgattctattaatgctgaagctactgtaactttacaaacttatactactcctaaaactagagaatatattgaaaaaaaatggggacaaaaaacttatacttatagaccttggggagtaaaaggaactgatgaatttttaatgggattatctgaattaactggaaaacctattcctaaagaatttgaaattgctagaggaagagctgtagatgctatgactgatactcaagcttgggtacatggaaaaagagctgctgtatatggagatcctgatttagtaatgggattattacaatttatgttagaaatgggaattgaacctgtacatgtattagtaaataattctactaaagaatttgaagaagaagctaaagctttattagctgcttctccttatggacaacaagctactgtatggggaggaaaagatttatggcatatgagatctttattatttactgaacctgtagattttttagtaggaaattcttatgctaaatatttacaaagagatactaaaactcctttaattagaattggatatcctatttttgatagacatcatttacatagatattctactattggatatgaaggagctattaatttattaaattggattgctaatggattaatggatgaattagatagaaaaactgatactccttctgtaactgatatttcttttgatttagtaagataggatccacctgatctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacgcggccgcattaccctgttatccctattaattaagagctcgctaccttaagagaggatatcggcgcgcctctagaatttaaatggatcctacgtactcgaggaattcaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaattcgggcacgaacccagtggacataagcctcgttcggttcgtaagctgtaatgcaagtagcgtaactgccgtcacgcaactggtccagaaccttgaccgaacgcagcggtggtaacggcgcagtggcggttttcatggcttcttgttatgacatgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctagctagaaattcgttcaagccgacgccgcttcgccggcgttaactcaagcgattagatgcactaagcacataattgctcacagccaaactatcaggtcaagtctgcttttattatttttaagcgtgcataataagccctacacaaattgggagatatatcatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgtcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgt

SEQ ID NO: 47 - DNA, 16728 bp, Artificial Sequence
  Plasmid DNA host by E.Coli Xl1B and or Agrobacteriun EHA105-transformed into plant cell
tgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatccaagctcaagctaagcttcagagccaccaccctcagagccgccaccagaaccaccaccagagccgccgccagcattgacaggaggcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggatcatccgggtctgtggcgggaactccacgaaaatatccgaacgcagcaagatatcgcggtccatctcggtcttgcctgggcagtcgccgccgacgccgttgatgtggacgccgggcccgatcatattgtcgctcaggatcgtggcgttgtgcttgtcggccgttgctgtcgtaatgatatcggcaccttcgaccgcctgttccgcagagatcccgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcaggtaccgcgatcgctcgcgacctgcaggcataaagccgtcagtgtccgcataaagaaccaacccataatacccataatagctgtttgccaaccggtcaacatgtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacgtcgagagttctcaacacaacatatacaaaacaaacgaatctcaagcaatcaagcattctacttctattgcagcaatttaaatcatttcttttaaagcaaaagcaattttctgaaaattttcaccatttacgaacgatagccatggctagcgctaccggactcagatctatggcttcctctgtcatttcttcagcagctgttgccacacgcagcaatgttacacaagctagcatggttgcacctttcactggtctcaaatcttcagccactttccctgttacaaagaagcaaaaccttgacatcacttccattgctagcaatggtggaagagttagctgcagagctcaaatgagacaaattgctttttatggaaaaggaggaattggaaaatctactacttctcaaaatactttagctgctatggctaatagacatggacaaagaattatgattgtaggatgtgatcctaaagctgattctactagattaattttaaatgctaaagctcaaactactgtattacatgtagctgctgaaagaggagctgtagaagatgtagaattagatgaagtattaaaacctggatttggaggaattaaatgtgtagaatctggaggacctgaacctggagtaggatgtgctggaagaggaattattactgctattaattttttagaagaagaaggagcttatactgatttagattttgtatcttatgatgtattaggagatgtagtatgtggaggatttgctatgcctattagagaaaataaagctcaagaaatttatattgtatgttctggagaaatgatggctatgtatgctgctaataatattgctagaggagtattaaaatatgctcatgctggaggagtaagattaggaggattaatttgtaattctagaaaagtagatagagaaactgaattaattgaaaatttagctgctagattaaatactcaaatgattcattttgtacctagagataatgtagtacaaagagctgaaattagaagaatgactgtagaacaatatgctcctgaagataatcaagctcaagaatatgatcaattagctcaaaaaattattaataatgaaaaattaactattcctactcctttagaaatggatgaattagaagaattattaattgaatttggattattaggagatgaagaagatagacaaaaacaaattgctgctcaagatgctgctattaaagctactgctgctaaataaggatccacctgatctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacgcggccgcacccataatacccataatagctgtttgccatcgctaccttaggaccgttatagttaaccggtcaacatgtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacgtcgagagttctcaacacaacatatacaaaacaaacgaatctcaagcaatcaagcattctacttctattgcagcaatttaaatcatttcttttaaagcaaaagcaattttctgaaaattttcaccatttacgaacgatagccatggctagcgctaccggactcagatctatggcttcctctgtcatttcttcagcagctgttgccacacgcagcaatgttacacaagctagcatggttgcacctttcactggtctcaaatcttcagccactttccctgttacaaagaagcaaaaccttgacatcacttccattgctagcaatggtggaagagttagctgcagagctcaaatggcttctgaaaaaattgaacaaaataaacaattaattcaagaagtattagatgcttatcctgctaaagctgctaaaagaagaaaaaaacatttaaatgtaattgaagaaaaaggagctgattgtggagtaaaatctaatgtaaaatctgtacctggagtaatgactactagaggatgtgcttttgctggagctaaaggagtagtatggggacctgtaaaagatatggtacatatttctcatggacctgtaggatgtggatattattcttgggctggaagaagaaattattataatggagtaactggagtagatacttttggaactatgcaatttacttctgattttcaagaaagagatattgtatttggaggagataaaaaattagctaaaattatgaatgaaattgaagaattatttcctttaaatgctggaattactattgaatctgaatgtcctgtaggattaattggagatgatattgaagctgtagctaaaaaagcttctaaagaattaaataaacctgtagtacctgtaagatgtgaaggatttagaggagtatctcaatctttaggacatcatattgctaatgatactgtaagagattgggtatatgaaccttctgctaaagtaactaatgaagaaattggatttgaaaaaactccttatgatgtatctttaattgctgattataatattggaggagatggatggtcttctagattattattagatgaaattggattaagagtagtatctcaagctactggagatggaacttataatgaagtatttatggctcctagagtaaatttaaatttaattcattgttatagatctatgaattatatttgtagatatatggaagaagaatatggaattccttgggtagaatttaatttttttggaccttctcaaattgctaaatctttaagaaaaattgcttctttttttgatgataaaattaaagaaaatactgaaaaagtaattgctagatatcaagaacaagctgatgctgtaattgctaaatatagacctagattagaaggaaaaaaagtaatgatgatggtaggaggattaagacctagacatattattcctgcttttgatgatttaggaatggaagtaattggaactggatatgaatttggacatggagatgattataaaagaactgctgattatgctcaagaaggaactttaatttatgatgatgtatctggatatgaatttgaagaatttgctaaaaaattaaaacctgatttaattgcttctggaattaaagaaaaatatgtatttcaaaaaatgggaatgccttttagacaaatgcattcttgggattattctggaccttatcatggatatgatggatttgctatttttgctagagatatggatttagctttaaattctcctacttggaatttaattaaagctccttggaaacaagctaaataggatccacctgatctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacgcggccgctcgctaccttaggaccgttatagttaattaccctgttatccctaaccggtcaacatgtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacgtcgagagttctcaacacaacatatacaaaacaaacgaatctcaagcaatcaagcattctacttctattgcagcaatttaaatcatttcttttaaagcaaaagcaattttctgaaaattttcaccatttacgaacgatagccatggctagcgctaccggactcagatctatggcttcctctgtcatttcttcagcagctgttgccacacgcagcaatgttacacaagctagcatggttgcacctttcactggtctcaaatcttcagccactttccctgttacaaagaagcaaaaccttgacatcacttccattgctagcaatggtggaagagttagctgcagagctcaaatgtctcaaaatgtagataaaattaaagatcattttcaattatttcaagaacctgaatatcaagaaatgtttgctagaaaaagagaatttgaaggaggagcttctaaagaagaaattgaaagagtaagagaatggactaaatcttgggaatatagagaaaaaaattttgctagagaagctttaactattaatcctgctaaagcttgtcaacctttaggagctatttttgctgctgctggatttgaaggaactttaccttttgtacatggatctcaaggatgtgtagcttattttagatctcatttaactagaaattataaagaaccttttcaagctgtatcttcttctatgactgaagatgctgctgtatttggaggattaaaaaatatgattgatggattagctaattcttatgctttatataaacctaaaatgattgctttatgtactacttgtatggctgaagtaattggagatgatttaggatcttttattactaattctaaaaatgaaggagctgtacctcaagattttcctgtaccttttgctcatactccttcttttgtaggatctcatattactggatatgataatatgttaaaaggaattttaattgctttaactgatggaaaaaaaactgaaactgataatggaaaaattaattttattcctggatttgatccttatattggaaatattagagatttaaaaaatattttatctttaatggatgtaccttctactgtattagctgataatgctgaatcttttgattctcctaatttaggagaatttaaaatgtataatggaggaactactttagaagaagctggagattctattaatgctaaagctactatttcttttcaaaaatattctactcctaaaactttagaatatttaaaacaagaaggaggacaaaaaactgctacttatagacctattggagtaagaggaactgatgaatttttaatggctttatctgaattaactggaaaagctattcctgaagaattagaaattgaaagaggaagagtagtagatgctattactgattctcaagcttggttacatggaaaaagaattgctatttatggagatcctgatcatgtattaggattattaaattttactttagaattaggaatgcaacctgtacatgtagtagtaaataatggaaatgtagctggatttgaagaagaagctaaagaattattagctaatgatcctaatggaaaagaagctactgtatggattggaaaagatttatggcatttaagatctttattagatactgaacctgtagatttattaattggaaattcttatggaaaatttttacaaagagatactggaactcctttagtaagaattggatatcctatttttgatagacatcatcaacatagatattctattttaggatataaaggagcttttaatttaattaattggattgtaaatactattttagatgaattagatagaggatctatggatttaggagtaaatgatacttcttttgatttagtaagataaggatccacctgatctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacgcggccgcattaccctgttatccctattaattaagagctcgctaccttaagagaggatatcggcgcgcctctagaatttaaatggatcctacgtactcgaggaattcaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaattcgggcacgaacccagtggacataagcctcgttcggttcgtaagctgtaatgcaagtagcgtaactgccgtcacgcaactggtccagaaccttgaccgaacgcagcggtggtaacggcgcagtggcggttttcatggcttcttgttatgacatgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctagctagaaattcgttcaagccgacgccgcttcgccggcgttaactcaagcgattagatgcactaagcacataattgctcacagccaaactatcaggtcaagtctgcttttattatttttaagcgtgcataataagccctacacaaattgggagatatatcatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgtcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgt

SEQ ID NO: 48 - DNA, 12181 bp, Artificial Sequence
  Plasmid DNA host by E.Coli Xl1B
ctgaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacccgattggggcgtggacataagggtctttatgacacaatcaacaattcgcttcattttcaattaggccttgctctagcttctttaggggttattacttctttggtagctcaacacatgtactctttacctgcttatgcattcatagcacaagactttactactcaagctgcattatatacccaccaccaatatatcgcaggattcatcatgacaggagcttttgctcatggagctatatttttcattagagattacaatccggagcaaaatgaagataatgtattggcaagaatgttagatcataaagaagctatcatatctcatttaagttgggccagcctctttctgggattccataccctgggactttatgttcataatgatgtcatgcttgcctttggcactccggagaagcaaatcttgattgaaccgatatttgctcaatggatacaatccgctcatggtaaaacttcatatgggttcgatgtacttttatcttcaacgactggcccagcattcaatgcgggtcgaagcatctggttgccgggttggttaaatgctgttaatgaaaatagtaattcattattcttaacaataggtcctggagactttttggttcatcatgctattgcccttggtttacatacaactacattgatcttagtaaaaggtgctttagatgcacgtggttccaagttaatgccagataaaaaggatttcggttatagttttccgtgcgatggcccaggacgaggcggtacttgtgatatttcggcatgggacgcgttttatttggcagttttttggatgttaaatactattggatgggttactttttattggcattggaagcacatcacattatggcaaggtaacgtttcacagtttaatgaatcttccacttatttgatgggctggttaagggattatttatggttaaactcttcacaacttatcaatggatataacccttttggtatgaatagtttatcggtttgggcatggatgttcttatttggacatcttgtttgggctactggatttatgttcttaatttcttggcgtggatattggcaggaattgattgaaactttagcatgggctcacgaacgcacacctttggccaatttgattcgatggagagataaaccagtggccctttctattgtacaagcaagattggttggattagctcacttttctgtaggttatatattcacttatgcggctttcttgattgcctctacgtcgggcaaatttggttaattaatgtgtgtattcgcgataatctcatttctttcgacggagaagggggtccaccttcttctatttctacatctaggattcgacttgtatcatggatactaataggaattcaaccattatggcaaggaaaagtttgattcagagggagaagaagaggcaaaaattggaacagaaatatcattcgattcgtcgatcctcaaaaaaagaaataagcaaggttccgtcgttgagtgacaaatgggaaatttatggaaagttacaatccctaccacggaatagtgcacctacacgccttcatcgacgttgttttttgaccggaaggccgagagctaactatcgagactttggcctatccggacacatacttcgtgaaatggttcatgcatgtttgttgccaggagcaacaagatcaagttggtaaggattaacgcttcatttctatttctatggtcgatgatcatagaagcccctttaccattctgtataaatgggctattctatttgtacagatagggtggaggggcgcatttaatccttgtttatctattagttttcagttcttatctttggcgcggggtagagcagtttggtagctcgcaaggctcataaccttgaggtcacgggttcaaatcctgtctccgcaacatcttattctggtctcgaggtccgcataaagaaccacccataatacccataatagctgtttgccaaccggtcgccgtcgttcaatgagaatggataagaggctcgtgggattgacgtgagggggcagggatgactatatttctgggagcgaactccgggcgaatatgaagcgcatggatacaagttatgccttggaatgaaagacaattccgaatccgctttgtctacgaacaaggaagctataagtaatgcaactatgaaggatctatgtctatcgataagaaaatccgtcaaatcgcattttacggaaaaggcggtatcggaaaatccactacctcacaaaacactcttgctgcaatggctgaaatgggtcaaagaatccttatcgttggatgcgatcctaaagcagacagtacccgccttatgcttcactctaaagctcaaaccaccgtacttcatcttgcagctgaacgtggtgctgttgaagatcttgaattagaggaagtaatgttaactggttttagaggagttaaatgcgtagagtctggtggtccagaaccaggtgttggatgtgcaggtcgaggaatcatcactgctatcaatttcttagaggaaaacggtgcatatcaagatgtagattttgtttcatacgatgttttaggtgatgtagtttgtggtggattcgctatgccgatccgtgagaacaaagcacaagaaatctatatcgtaaccagtggtgagatgatggctatgtacgcagctaataacatcgctagaggtatcttaaagtatgcacatactggaggggttcgattaggcgggttaatctgtaattcacgtaacgtagatcgtgaaatcgagttgatcgaaactttggctaaaagattgaatactcaaatgatccattacgttcctcgcgataacatcgtacaacatgccgagttgcgtcgcatgacagtaaatgaatatgcccctgattccaatcagtctaatgagtatcgtatcttggcgaataaaatcatcaataatgaaaatttgaaagtacctactcctatcgagatggaagaactagaagaactactgatcgaatttggtatcctggaatctgaagaaaatgctgctaaaatgatcgggactcctgctcagtcttccactaaataatagcggccgcaacccataatacccataatagctgtttgccatcgctaccttaggaccgttatagttaaccggtggaggcagactatgactcctccagaaaatcaaaacctagtagatgagaataaagagttaatcaaagaagtgcttcaagcttaccctgaaaaggcacgaaaaaaaagagagaagcatttaaatgttcacgaagaaagtaaatccgattgtggtgtaaaatctaacataaagtccattccaggcgttatgactgctcgtgggtgcgcatatgctggtagcaaaggggtagtttggggtcctatcaaagatatgattcatatatcccacgggcctgtaggttgtgggtactggtcttggtccggaagacgtaattattacgttggtatcaccggaattaactctttcggtactatgcattttacttccgatttccaggaaagagacatcgtatttggaggtgataagaaattgaccaaaataattgatgagttagaaatcctttttccattaaatcgtggagtgtctattcaatcagaatgccctatcggtttgattggaggtgatatcgaagcagttgctaaaaaagcatctaagcagtatggaaaaccagtagttcctttaagatgtgagggtctgcgtggagtatcacaatctttaggtcatcacattgctaacgacgcaatcagagattggattttcccagaatttgataaggctaaaaaaaataatactatcgactttgaaccttcaccgtacgatgttgcacttattggagatcataacatcggtggagatgcttgggcatctcgtatgttattggaggaaatgggtctgagagtagttgctcaatggtcaggagatggtactttaaatgaacttattcaaggacctgcagctaaattggtactgattcactgctatcgttctatgaattacatctgtcgctcattggaggaacaatatggtatgccgtggatggaattcaacttttttggacctactaaaattgcagcttctcttcgtgagatcgcagctaaattcgacagtaaaattcaagaaaatgcagaaaaagttatcgctaaatacaccccggtaatgaatgcagtgcttgagaaatatcgccctcgtttggaaggtaacactgttatgctttacgtagggggtcttcgccctcgtcatgttgtacctgctttcgaagatttggggattaaagttgtaggtaccgggtatgaatttgcacataatgatgattacaaacgcactacacactatatcgataacgctactattatctacgatgatgttacagcctatgagttcgaagaatttgtaaaagctaaaaaacctgatcttattgcctctggtatcaaagaaaaatacgcttttcagaaaatgggtctaccttttcgacaaatgcatagttgggattattctggtccttatcatggttatgatcgttttgccatctttgctcgagatatggatcttgccctaaatagtcctacttggagtctaatcggtgcgccttggaaaaaagcggctactaaagcgaaagctgctgcttaataactcgagagatacaacaatgcctcaaaatccagagaaaattgtagatcatgtacaattatttcagcaacctgaatatcaagaacttttcaaaaataaacacgaacaatttgagggagctcattccccagaagaagtacagagagtatccgaatggactaaaggttgggagtacagagaaaagaactttgcacgtcaagctttaacagtaaatcctgcaaaaggatgccaaccagtaggtgctatcttcgcagctgtaggatttgaaggtactctacctttcgtacaaggatcccagggttgtgtagcatattttagaacacatttatcccgtcactacaaagaaccattctctgctgtatcctcatctatgactgaggatgcagctgtatttggaggtcttaacaatatgattgaaggattacaagtatcatatcaactatacaaacctaaaatgatcgcagtatgtacaacttgtatggctgaagtaattggtgatgatttaggagcattcatcacaaatgctaaaaacgcaggttctattccacaagattttcctgtaccattcgctcatactccttcatttgtaggatctcatacaaccggttatgataatatgatgaagggaatcctttcaaatttaactgagggtaaaaaaaaagataagaccaacggaaaaattaatttcatcccaggttttgatacttacgtaggaaataacagagaactgaaacgtatgttaggtcttatgggaattgattataccatcttagcagataattctgattacctggatgctcctaatactggtgaatatgatatgtacccgggcggtaccaaattagaggatgcagctgattcatgcaacgcagaagctactgtagcccttcagagatataccactaccaagactcgagaatacattgagaccaaatggaaacaacaaactaaagtattacgtcctttcggggtaaaaggtaccgacgaatttcttatgactttatctgaaatgaccgggaaaccgatccctcaagaacttgaggatgaacgaggtcgtttagtagacgctatgactgatagttatgcctggattcacgggaaaaaattcgctatctacggtgatccggaccttatttattctgtaaccagttttttacttgaattaggggccgaacctgtacatatcctttgtaataatggtgatgaagagttcaaaaaagagatggaagctattttatctgcgagtccgtttgggaaacaagctactgtatggatccagaaagatttgtggcatcttcgatctttgctttttactgaacctgtagatttttttgtaggtaacagttacgggaaatatttgtggcgcgacaccaaaattcctatggtacgtatcggttatcctctttttgatcgccaccatttgcatcgttatcctactttggggtatcaaggtggtttgaatttgcttaattgggtagtaaacactattttggatgaacttgatcgcagtagcaatatagcgggtaaaactgatatatcttttgatttgatccgttaataggcggccgctcgctaccttaggaccgttatagttattaccctgttatccctaaccggtggaggcttcttgttatgacatgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatctcgagctcaagcttcgaattctgcagtcgacggtaccgcgggcccgggatccacctgatctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacgcggccgcattaccctgttatccctattaattaagagctcgctaccttaagagaggatatcggcgcgccgatcctagcctagtctataggaggttttgaaaagaaaggagcaataatcattttcttgttctgtcaagagggtgctattgctcctttctttttttttttttctttactaatttcctagtattttactgacatagacttttttgtttacattatcgaaaaagaaagagagggtatttgcttgcatttattcatgatggatcccccgggctgcaggcatgcaagctaattcccgatctagtaacatagatgacaccgcgcgccgcggaactagtaattaattcccgcctttcgctttttgggggtggaaggcaaaagaaaacgtaggggagggatagaatcactacactatcacggccaactataccaactccttaatgtaaggatatatttaatgctatttatgaaattcaataataaaaagaaatagtaaaaaaattactttatcttggatcttgggcggatagcgggaatcgaacccgcatcttctccttggcaaagagaaattttaccattcgaccatatccgcatttttttgttcttgatacacaatatgtacccacatatatgatatataaccggatcttatttgtgcagtgccgggacacatattctcttcggaacgattccaataattttgttaattatattctttttattcaagaagtttgacccccctctaatttttttgttttctttatttgatttgcattttctttggggacttagattcaaatttaatgtgtctcacaaccgagaaaaattaggggggtcattttggttttgggtctgcgacgaataggttcaagagatgagagaattaaggatacccaccagaaagactaatccaatccataaggaggtaccagaaaatacaacatttttgttacttgaccagccatcaggagaagcaaatacaacgggtacgctaatcaataagattaatgaagtagcaattaatgcaaaaacagccaattggaaagcaagagtcatgcttttaatcctccaagctaccaacaaatgaactatataccatttgatccctctatcagccaaaaaatattaattgtgataaaatatgtcatcgagggattttactttatcatgaatccattgattctatatgacttattactactccccctttcgcactttattcgtacatggagtggggtggggggaaatggaattttcttttttatttcacaaatggacatgctagatcatatatctatatacggatagatagatcgatcggcggattcgcacctgagatctttctacagatagtgggggtatccacccctatagccatgttctattcggaggaataaaataaaaatagtctttcggagagatggctgagtggttgatagccccggtcttgaaaaccggtatagttttgaacaaagaactatcgagggttcgaatccctctctctccttttttgctaattgaatagatttttttatttagtggttttgcccaatctgctatccgaaagaaaagggaatggctcggctatcccacctagccaagccagaaaaatagattagatataaattagataaaataaatgagttgaaaaaaaaaaaagaaaaaaggaatacttaagctgattccaagatgtatgattgaatcaaagtaatttgtacttcattcaagcattggatctcctgtctcatatcaattaagaggggtcatggaaagaacaggttcaaagtcgcgatcaattcctttttcaaatcctgctgcagcgagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 49 - DNA, 16791 bp, Artificial Sequence
  Plasmid DNA host by E.Coli Xl1B
tgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatccaagctcaagctaagcttcagagccaccaccctcagagccgccaccagaaccaccaccagagccgccgccagcattgacaggaggcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggatcatccgggtctgtggcgggaactccacgaaaatatccgaacgcagcaagatatcgcggtccatctcggtcttgcctgggcagtcgccgccgacgccgttgatgtggacgccgggcccgatcatattgtcgctcaggatcgtggcgttgtgcttgtcggccgttgctgtcgtaatgatatcggcaccttcgaccgcctgttccgcagagatcccgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcaggtaccgcgatcgctcgcgacctgcaggcataaagccgtcagtgtccgcataaagaaccaacccataatacccataatagctgtttgccaaccggtcaacatgtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacgtcgagagttctcaacacaacatatacaaaacaaacgaatctcaagcaatcaagcattctacttctattgcagcaatttaaatcatttcttttaaagcaaaagcaattttctgaaaattttcaccatttacgaacgatagccatggctagcgctaccggactcagatctatggcttcctctgtcatttcttcagcagctgttgccacacgcagcaatgttacacaagctagcatggttgcacctttcactggtctcaaatcttcagccactttccctgttacaaagaagcaaaaccttgacatcacttccattgctagcaatggtggaagagttagctgcagagctcaagcttcgaattctatgtctatcgataagaaaatccgtcaaatcgcattttacggaaaaggcggtatcggaaaatccactacctcacaaaacactcttgctgcaatggctgaaatgggtcaaagaatccttatcgttggatgcgatcctaaagcagacagtacccgccttatgcttcactctaaagctcaaaccaccgtacttcatcttgcagctgaacgtggtgctgttgaagatcttgaattagaggaagtaatgttaactggttttagaggagttaaatgcgtagagtctggtggtccagaaccaggtgttggatgtgcaggtcgaggaatcatcactgctatcaatttcttagaggaaaacggtgcatatcaagatgtagattttgtttcatacgatgttttaggtgatgtagtttgtggtggattcgctatgccgatccgtgagaacaaagcacaagaaatctatatcgtaaccagtggtgagatgatggctatgtacgcagctaataacatcgctagaggtatcttaaagtatgcacatactggaggggttcgattaggcgggttaatctgtaattcacgtaacgtagatcgtgaaatcgagttgatcgaaactttggctaaaagattgaatactcaaatgatccattacgttcctcgcgataacatcgtacaacatgccgagttgcgtcgcatgacagtaaatgaatatgcccctgattccaatcagtctaatgagtatcgtatcttggcgaataaaatcatcaataatgaaaatttgaaagtacctactcctatcgagatggaagaactagaagaactactgatcgaatttggtatcctggaatctgaagaaaatgctgctaaaatgatcgggactcctgctcagtcttccactaaataaggatccacctgatctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacgcggccgcacccataatacccataatagctgtttgccatcgctaccttaggaccgttatagttaaccggtcaacatgtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacgtcgagagttctcaacacaacatatacaaaacaaacgaatctcaagcaatcaagcattctacttctattgcagcaatttaaatcatttcttttaaagcaaaagcaattttctgaaaattttcaccatttacgaacgatagccatggctagcgctaccggactcagatctatggcttcctctgtcatttcttcagcagctgttgccacacgcagcaatgttacacaagctagcatggttgcacctttcactggtctcaaatcttcagccactttccctgttacaaagaagcaaaaccttgacatcacttccattgctagcaatggtggaagagttagctgcagagctcaaatgactcctccagaaaatcaaaacctagtagatgagaataaagagttaatcaaagaagtgcttcaagcttaccctgaaaaggcacgaaaaaaaagagagaagcatttaaatgttcacgaagaaagtaaatccgattgtggtgtaaaatctaacataaagtccattccaggcgttatgactgctcgtgggtgcgcatatgctggtagcaaaggggtagtttggggtcctatcaaagatatgattcatatatcccacgggcctgtaggttgtgggtactggtcttggtccggaagacgtaattattacgttggtatcaccggaattaactctttcggtactatgcattttacttccgatttccaggaaagagacatcgtatttggaggtgataagaaattgaccaaaataattgatgagttagaaatcctttttccattaaatcgtggagtgtctattcaatcagaatgccctatcggtttgattggaggtgatatcgaagcagttgctaaaaaagcatctaagcagtatggaaaaccagtagttcctttaagatgtgagggtctgcgtggagtatcacaatctttaggtcatcacattgctaacgacgcaatcagagattggattttcccagaatttgataaggctaaaaaaaataatactatcgactttgaaccttcaccgtacgatgttgcacttattggagatcataacatcggtggagatgcttgggcatctcgtatgttattggaggaaatgggtctgagagtagttgctcaatggtcaggagatggtactttaaatgaacttattcaaggacctgcagctaaattggtactgattcactgctatcgttctatgaattacatctgtcgctcattggaggaacaatatggtatgccgtggatggaattcaacttttttggacctactaaaattgcagcttctcttcgtgagatcgcagctaaattcgacagtaaaattcaagaaaatgcagaaaaagttatcgctaaatacaccccggtaatgaatgcagtgcttgagaaatatcgccctcgtttggaaggtaacactgttatgctttacgtagggggtcttcgccctcgtcatgttgtacctgctttcgaagatttggggattaaagttgtaggtaccgggtatgaatttgcacataatgatgattacaaacgcactacacactatatcgataacgctactattatctacgatgatgttacagcctatgagttcgaagaatttgtaaaagctaaaaaacctgatcttattgcctctggtatcaaagaaaaatacgcttttcagaaaatgggtctaccttttcgacaaatgcatagttgggattattctggtccttatcatggttatgatcgttttgccatctttgctcgagatatggatcttgccctaaatagtcctacttggagtctaatcggtgcgccttggaaaaaagcggctactaaagcgaaagctgctgcttaaggatccacctgatctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacgcggccgctcgctaccttaggaccgttatagttaattaccctgttatccctaaccggtcaacatgtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacgtcgagagttctcaacacaacatatacaaaacaaacgaatctcaagcaatcaagcattctacttctattgcagcaatttaaatcatttcttttaaagcaaaagcaattttctgaaaattttcaccatttacgaacgatagccatggctagcgctaccggactcagatctatggcttcctctgtcatttcttcagcagctgttgccacacgcagcaatgttacacaagctagcatggttgcacctttcactggtctcaaatcttcagccactttccctgttacaaagaagcaaaaccttgacatcacttccattgctagcaatggtggaagagttagctgcagagctcaagcttcgaattctatgcctcaaaatccagagaaaattgtagatcatgtacaattatttcagcaacctgaatatcaagaacttttcaaaaataaacacgaacaatttgagggagctcattccccagaagaagtacagagagtatccgaatggactaaaggttgggagtacagagaaaagaactttgcacgtcaagctttaacagtaaatcctgcaaaaggatgccaaccagtaggtgctatcttcgcagctgtaggatttgaaggtactctacctttcgtacaaggatcccagggttgtgtagcatattttagaacacatttatcccgtcactacaaagaaccattctctgctgtatcctcatctatgactgaggatgcagctgtatttggaggtcttaacaatatgattgaaggattacaagtatcatatcaactatacaaacctaaaatgatcgcagtatgtacaacttgtatggctgaagtaattggtgatgatttaggagcattcatcacaaatgctaaaaacgcaggttctattccacaagattttcctgtaccattcgctcatactccttcatttgtaggatctcatacaaccggttatgataatatgatgaagggaatcctttcaaatttaactgagggtaaaaaaaaagataagaccaacggaaaaattaatttcatcccaggttttgatacttacgtaggaaataacagagaactgaaacgtatgttaggtcttatgggaattgattataccatcttagcagataattctgattacctggatgctcctaatactggtgaatatgatatgtacccgggcggtaccaaattagaggatgcagctgattcatgcaacgcagaagctactgtagcccttcagagatataccactaccaagactcgagaatacattgagaccaaatggaaacaacaaactaaagtattacgtcctttcggggtaaaaggtaccgacgaatttcttatgactttatctgaaatgaccgggaaaccgatccctcaagaacttgaggatgaacgaggtcgtttagtagacgctatgactgatagttatgcctggattcacgggaaaaaattcgctatctacggtgatccggaccttatttattctgtaaccagttttttacttgaattaggggccgaacctgtacatatcctttgtaataatggtgatgaagagttcaaaaaagagatggaagctattttatctgcgagtccgtttgggaaacaagctactgtatggatccagaaagatttgtggcatcttcgatctttgctttttactgaacctgtagatttttttgtaggtaacagttacgggaaatatttgtggcgcgacaccaaaattcctatggtacgtatcggttatcctctttttgatcgccaccatttgcatcgttatcctactttggggtatcaaggtggtttgaatttgcttaattgggtagtaaacactattttggatgaacttgatcgcagtagcaatatagcgggtaaaactgatatatcttttgatttgatccgttaagatccacctgatctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacgcggccgcattaccctgttatccctattaattaagagctcgctaccttaagagaggatatcggcgcgcctctagaatttaaatggatcctacgtactcgaggaattcaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaattcgggcacgaacccagtggacataagcctcgttcggttcgtaagctgtaatgcaagtagcgtaactgccgtcacgcaactggtccagaaccttgaccgaacgcagcggtggtaacggcgcagtggcggttttcatggcttcttgttatgacatgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctagctagaaattcgttcaagccgacgccgcttcgccggcgttaactcaagcgattagatgcactaagcacataattgctcacagccaaactatcaggtcaagtctgcttttattatttttaagcgtgcataataagccctacacaaattgggagatatatcatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgtcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgt

(19) United States
(12) Patent Application Publication
(10) Pub. No.: US 2014/0196178 A1
(43) Pub. Date: Jul. 10, 2014

(54) PLANT SELF NITROGEN FIXATION BY MIMICKING PROKARYOTIC PATHWAYS

(71) Applicant: Adi Zaltsman, Port Jefferson, NY (US)

(72) Inventor: Adi Zaltsman, Port Jefferson, NY (US)

(21) Appl. No.: 14/093,920

(22) Filed: Dec. 2, 2013

Related U.S. Application Data
(60) Provisional application No. 61/858,218, filed on Jul. 25, 2013.
(60) Provisional application No. 61/732,490, filed on Dec. 3, 2012.

(51) Int. Cl. C12N 15/82

(52) U.S. Cl. CPC C12N 15/8243
USPC 800/306; 800/298; 800/320.1; 800/320.2; 800/317.3; 800/312; 800/314; 800/317.4; 800/320.3; 800/317.2; 800/320; 426/549; 426/618; 426/622; 131/360; 131/352

(57) ABSTRACT

A method for engineering a transgenic plant that replicates the nitrogen fixation mechanism of photosynthetic bacteria, such as cyanobacteria, or that of other bacteria, by targeting to or expressing in plant plastids bacterial nif (nitrogen fixation) genes is provided. A method for reducing the overall concentration of nitrogen in soil is provided using a plant that replicates the nitrogen fixation mechanism of photosynthetic bacteria. Progeny of a plant that replicates the nitrogen fixation mechanism of photosynthetic bacteria is also provided.

### CROSS REFERENCE TO RELATED APPLICATIONS

This application claims priority to U.S. Provisional Application No. 61/858,218 filed on Jul. 25, 2013 and U.S. Provisional Application No. 61/732,490 filed Dec. 3, 2012, which are each incorporated herein by reference, in its entirety.

### FIELD OF THE INVENTION

The present disclosure relates to a novel transgenic plant that replicates the nitrogen fixation mechanism of photosynthetic bacteria, such as, for example, cyanobacteria or other bacteria by targeting or expressing bacterial nif (nitrogen fixation) genes to produce the structural proteins of the enzyme nitrogenase and by introducing synthetic DNA sequences coding for proteins homologous to the structural proteins of nitrogenase and methods of producing such a plant.

### BACKGROUND

Plants are living organisms belonging to the kingdom Plantae. All higher plants have the vascular tissues xylem (mostly water and mineral transports) and phloem (mostly sugar and other metabolites transport). Vascular plants include all seed-bearing plants (gymnosperms and angiosperms) and the pteridophytes (including ferns, lycophytes, and horsetails), which are also called tracheophytes. These are eukaryotes having an organized cell structure that includes a nucleus (in most cells) and chloroplasts (or other types of plastids) having the ability to use light, such as sunlight, as an energy source for carbon fixation during photosynthesis. Other types of plants include, for example, algae, mosses and fungi.

The general limiting factor for crop productivity of agricultural crops is the nitrogen content in soil and water. The supply of this element has been reduced over time as agricultural production attempts to keep pace with the increased demands of a growing population and the reduced availability of farmland. Nitrogen is one of the primary nutrients essential to all forms of life, including plants. However, nitrogen must first be converted to a form that plants can utilize. The phenomenon of Biological Nitrogen Fixation (BNF) is the conversion of atmospheric nitrogen (N₂) to ammonia (NH₃) using the enzyme nitrogenase. BNF is usually represented by the chemical equation: N₂+8H⁺+8e⁻+16ATP>>>2NH₃+H₂+16ADP+16Pi, or by saying that a nitrogen gas molecule has been reduced to two molecules of ammonia in the presence of eight protons and eight electrons that are consuming sixteen molecules of ATP (Adenosine TriPhosphate—the cell's energy molecule). This reaction consumes a tremendous amount of energy as N₂ contains a triple bond. The bond energy in a nitrogen molecule is about 225 kcal/mol. Few BNFs are performed in nature as a result of a symbiotic relationship between plants and several bacterial species that make up a “nitrogenase enzymatic complex.” The nitrogenase enzymatic complex consists of two proteins: a Fe-protein (an enzyme known as Nif-H) and a Mo—Fe protein (α and β subunits known as Nif-D-K). The nitrogenase complex is composed of a heterotetrameric (not the same units) MoFe (iron-molybdenum cofactor) protein that is transiently associated with a homodimeric (at least two of the same unit) Fe (iron cofactor) protein.

The Nif-H genes encode the iron protein and the Nif-D and K genes encode the molybdenum iron protein. Accordingly, Nif-D and Nif-K genes require Mo and Fe as cofactors in their final active form. Nif-D encodes the Nif-D protein, also known as an alpha subunit and the Nif-K encodes the Nif-K protein is known as a beta subunit. In other words, Nif-H is a dimer enzyme with 2 identical subunits (a total of 2 proteins), while Nif-D-K is a 2α2β dimer (a total of 4 proteins). All six subunits are essential and are required for its function. In nature, Nif-H has a great variety and contributes to biodiversity. The fact that there are multiple types of Nif-H provides the ability for adaptation to various natural conditions.

The bacterial species that produce the nitrogenase enzymatic complex include diazotrophs such as cyanobacteria, azotobacteraceae, rhizobia, and frankia. For example, in cyanobacteria, the Mo—Fe protein (Nif-D-K) binds atmospheric nitrogen (N₂) and the Fe-protein (Nif-H) then reduces it by an electron that is donated by ferredoxin. Ferredoxins are proteins that function as electron carriers in the photosynthetic electron transport chain that is similar, but not identical to the higher plant chloroplasts, Fe₂S₂ ferredoxins. The reduced Fe-protein will use ATP to transfer the (reduced) electron to the Mo—Fe protein that will then donate the electron to N₂ (nitrogenase reductase). By repeating this process several times, all three covalent bonds of N═N are reduced to 2NH₃.

However, only a few plant species can live in a symbiotic relationship with diazotrophs. For example, the pea plant from the legume family lives in symbiosis with bacteria from the rhizobia family. In particular, rhizobia bacteria penetrate the pea plant's roots creating root nodules that contain bacteria that fix nitrogen (to ammonia) while the plant donates carbon (sugar). Improving either the symbiosis, or extending the host range would therefore be beneficial for plant survival, but achieving this goal includes many challenges including the complexity of the process and lack of basic knowledge.

Nitrogen can also be fixed chemically using an artificial process. This method of fixing nitrogen is most commonly produced through a heat reaction known as the Harber process. This process requires high pressure and temperature for a relatively simple reaction. For the last hundred years, the demand for nitrogen fertilizer has steadily increased to more than 200 million metric tons per year. This consumption is likely to increase.

A non-symbiotic organism (e.g., a free living-organism that has no established symbiotic relationship with any microorganism to fix nitrogen) can accomplish self Biological Nitrogen Fixation (sBNF) if they each included the core enzyme nitrogenase. The existence of non-bacterial organisms like crop plants and algae and other plants that are capable of self-nitrogen fixation would be useful for several purposes such as reducing fertilization needs, reducing fertilization pollution, providing an eco-friendly crop production, enhanced crop production, improved oil content in plants, improved protein content in plants, the reduction of nitrogen contamination of water, and the enrichment of the carbon content relative to nitrogen and carbon in relation to a soil's organic phase. However, there are no known plants that are able to achieve these results for several reasons, one of which is the particular enzymatic complex of nitrogenase, which includes several proteins and genes for nitrogen fixation.

Almost all core enzymes that are used for atmospheric nitrogen fixation are very sensitive to oxygen. This characteristic makes it almost impossible to fix nitrogen in plants, because plants generate oxygen from water during the photosynthesis reaction. Cyanobacteria are considered to be the evolutionary ancestor (foundation) of chloroplasts. While the chloroplast has lost many of its original genes during evolution, cyanobacteria maintain many of the genes that the chloroplast lost. Cyanobacteria inhabit nearly all illuminated environments on Earth as photosynthetic organisms. They play a key role in the Biological Nitrogen Fixation (BNF) process facilitated by more than 20 nitrogen fixation (NIF) genes. Only three of these genes (Nif-D, Nif-K and Nif-H) are the NIF enzymes (nitrogenase). The rest of the NIF genes are involved in the complex assembly, process of the cofactors, and controlling of expression.

Leguminous crop plants such as soy, beans, or peas are well known for their unique ability to develop root nodules occupied by Rhizobium sp., symbiotic bacteria. These bacteria are capable of BNF and donation of ammonia to the plant. Most crop plants do not have this ability and the Rhizobium sp. will fail to interact with them. Several unpublished attempts were made to extend the “host” range of the Rhizibia, all of which failed.

Thus, what is needed is a system and method that includes transgenic plants that are capable of cellular fixation of nitrogen to produce ammonia at a significant rate that would make the system sustainable and commercially viable. The sections below describe such a system.

### SUMMARY

The present disclosure is directed to transgenic plants (genetically modified organisms or (CMOs)) transformed to be able to perform the process of auto/self fixation of nitrogen to produce their own usable source of nitrogen thereby reducing dependency on nitrogenous fertilizers as a source of nitrogen. The present disclosure also refers to plant cells, tissues, parts of plants or plant lines comprising the genes to transform these plants to enable them to perform the auto/self fixation of nitrogen.

In some embodiments, in accordance with the principles of the present disclosure, a plant exhibiting a modified self/auto nitrogen fixating profile is provided that is produced by a method comprising the steps of: introducing one or more plant cells with a recombinant nucleic acid sequence encoding Nif-H, Nif-D and Nif-K genes operatively linked to a promoter sequence and a terminator sequence; regenerating one or more plants from the plant cells and selecting one or more plants, cultivated from the plant cells, exhibiting enhanced nitrogen fixation such that one or more plants comprise the recombinant nucleic acid sequence encoding Nif-H, Nif-D and Nif-K genes. In some embodiments, the recombinant nucleic acid sequence encoding Nif-H, Nif-D and Nif-K genes is obtained from a single organism. In some embodiments, the recombinant nucleic acid sequence encoding Nif-H, Nif-D and Nif-K genes is obtained from different organisms. For example, in some embodiments, the recombinant nucleic acid sequence encoding at least one of the Nif-H, Nif-D and Nif-K genes is obtained from a first organism and the recombinant nucleic acid sequence encoding at least one of the Nif-H, Nif-D and Nif-K genes is obtained from a second organism that is different than the first species. In some embodiments, the recombinant nucleic acid sequence encoding Nif-H, Nif-D and Nif-K genes occurs in nature. In some embodiments, the recombinant nucleic acid sequence encoding Nif-H, Nif-D and Nif-K genes is synthetic and is not found in nature.

In some embodiments, in accordance with the principles of the present disclosure, a plant exhibiting a modified self/auto nitrogen fixating profile is provided that is produced by a method comprising the following steps: contacting a plant cell with a recombinant nucleic acid sequence encoding one of SEQ. ID. NO. 1, SEQ. ID. NO. 2 and SEQ. ID. NO. 3; regenerating one or more plants from the plant cell; and selecting one or more plants cultivated from the plant cell, wherein the selected plants each exhibit enhanced nitrogen fixation, wherein the selected one or more plants each comprise the recombinant nucleic acid sequence encoding SEQ. ID. No. 1, SEQ. ID. No. 2, SEQ. ID. NO. 3, SEQ. ID. NO. 46, SEQ. ID. NO. 47, SEQ. ID. NO. 48 or SEQ. ID. NO 49.

In some embodiments, in accordance with the principles of the present disclosure, a plant exhibiting enhanced nitrogen fixatation is provided that is produced by a method comprising the steps of: contacting a plant cell with a recombinant nucleic acid sequence encoding one of SEQ. ID. NO. 1, SEQ. ID. NO. 2, SEQ. ID. NO. 3, SEQ. ID. NO 46, SEQ. ID. NO 47, SEQ. ID. NO 48 or SEQ. ID. NO. 49; contacting the plant cell with at least one synthetic coding DNA sequence (sCDS) optimized for tomato chloroplast expression; regenerating one or more plants from the plant cell; and selecting one or more plants, cultivated from the plant cell exhibiting enhanced nitrogen fixation, wherein said one or more plants comprise the recombinant nucleic acid sequence encoding SEQ. ID. NO. 1, SEQ. ID. NO. 2, SEQ. ID. NO. 3, SEQ. ID. NO 46, SEQ. ID. NO 47, SEQ. ID. NO 48 or SEQ. ID. NO. 49 and the sCDS.

In some embodiments, in accordance with the principles of the present disclosure, a plant exhibiting enhanced nitrogen fixatation is provided that is produced by a method comprising the steps of: contacting one or more plant cells with a recombinant nucleic acid sequence encoding a Chimera Nif gene with plant transit peptide or signal for the chloroplast (plasid) accmolation as, Nif-H gene operatively linked to a first promoter; contacting the plant cells with a recombinant nucleic acid sequence encoding a Nif-D gene operatively linked to a second promoter; contacting the plant cells with a recombinant nucleic acid sequence encoding a Nif-K gene operatively linked to a third promoter; regenerating one or more plants from the plant cells; and selecting one or more plants cultivated from the plant cells, wherein the selected plants each exhibit enhanced nitrogen fixation, wherein the selected plants each comprise the recombinant nucleic acid sequence encoding the Nif-H gene, the recombinant nucleic acid sequence encoding the Nif-D gene and the recombinant nucleic acid sequence encoding the Nif-K gene. In some embodiments, the Nif-H gene is from a group consisting essentially of SEQ ID. NO. 29, SEQ ID. NO. 32, SEQ ID. NO. 35 and SEQ ID. NO. 38. In some embodiments, the Nif-D gene is selected from a group consisting essentially of SEQ ID. NO. 30, SEQ ID. NO. 33, SEQ ID. NO. 36 and SEQ ID. NO. 39. In some embodiments, the Nif-K gene is from a group consisting essentially of SEQ ID. NO. 31, SEQ ID. NO. 34, SEQ ID. NO. 37 and SEQ ID. NO. 40. In some embodiments, the first, second and third promoters are selected from a group consisting essentially of SEQ. ID. NOs. 41-45.

The present disclosure is also directed to a transgenic plant-derived commercial product, which is derived from a transgenic plant according to method described in the present disclosure.

A method for reducing the overall concentration of nitrogen in soil is also described according to the principles of the present disclosure. The method comprises placing at least one transgenic plant described in the present disclosure in contact with soil in which the level of nitrogen is to be reduced; and allowing the plant to grow and fix nitrogen obtained from the soil for their metabolic results in reducing the overall concentration of nitrogen in the soil. These and other principles are further described using the figures and in the Detailed Description.

### BRIEF DESCRIPTION OF THE DRAWINGS

FIG. 1 shows the schematic structure of nitrogenase complex.

FIG. 2 shows a schematic representation of the steps used for producing a plasmid configured to be inserted into a plastid during tomato chloroplast transformation.

FIG. 3 shows a schematic representation of the steps for producing the plasmid for tomato chloroplast transformation and the expression of the Nif-H, Nif-D, and Nif-K genes (PGE 011).

FIG. 4 shows Nif-H, Nif-D and Nif-K genes cloned as a single operon and inserted into a C. reinhardtii chloroplast.

FIG. 5 shows the development of tomato plants at three weeks post-germination.

FIG. 6 shows the accumulation of Nif-H in plant plasmids.

FIG. 7 shows the schematic structure of the plant transformation plasmids.

### DETAILED DESCRIPTION

The present disclosure is directed to transgenic plants enabled to fix nitrogen, products produced from such plants, a method of producing the transgenic plants and a method of reducing nitrogen in soil or water using the transgenic plants.

The present disclosure may be understood more readily by reference to the following detailed description of the disclosure taken in connection with the accompanying drawing figures, which form a part of this disclosure. It is to be understood that this disclosure is not limited to the specific compositions, devices, methods, conditions or parameters described and/or shown herein, and that the terminology used herein is for the purpose of describing particular embodiments by way of example only and is not intended to be limiting of the claimed disclosure. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

For the purposes of this specification and appended claim, unless otherwise indicated, all numbers expressing quantities of ingredients, percentages or proportions of materials, reaction conditions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques.

Notwithstanding the numerical ranges and parameters set forth herein, the broad scope of the present disclosure are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a range of “1 to 10” includes any and all subranges between (and including) the minimum value of 1 and the maximum value of 10, that is, any and all subranges having a minimum value of equal to or greater than 1 and a maximum value of equal to or less than 10, e.g., 5.5 to 10.

The headings in this application are not meant to limit the disclosure in any way; embodiments under any one heading may be used in conjunction with embodiments under any other heading.

It is noted that, as used in this specification and the appended claims, the singular forms “a,” “an,” and “the,” include plural referents unless expressly and unequivocally limited to one referent. Thus, for example, reference to “an intervertebral disc implant” includes one, two, three or more intervertebral disc implants.

The following discussion includes a description of compositions, genetic procedures, associated transgenic cells necessary to transform plants to be able to fix their own nitrogen and the resulting transgenic plants, as well as, methods of using transgenic plants (genetically modified organisms (GMOs) to reduce the need or demand for nitrogen fertilization in accordance with the principles of the present disclosure. Alternate embodiments are also disclosed. Reference will now be made in detail to certain embodiments of the present disclosure, examples of which are illustrated in the accompanying drawings and discussed in the examples. While the present disclosure will be described in conjunction with the illustrated embodiments, it will be understood that they are not intended to limit the present disclosure to those embodiments. On the contrary, the present disclosure is intended to cover all alternatives, modifications, and equivalents that may be included within the present disclosure, as defined by the appended claims.

Turning now to the composition and methods of the present disclosure, the compositions and methods provided herein are for the production of organic nitrogen in the plant through activity of heterologous nitrogenase, so the plant can produce ammonia.

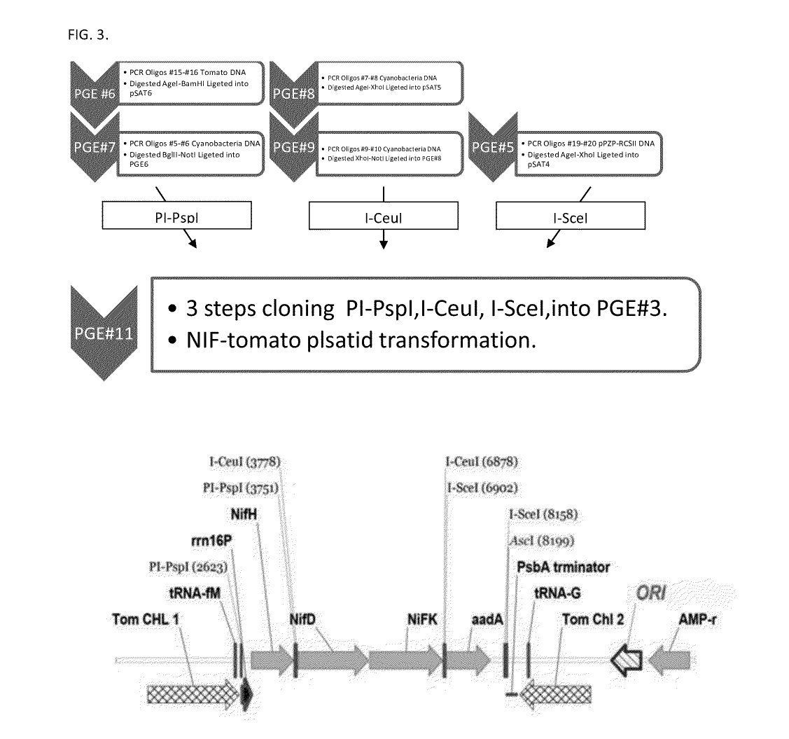

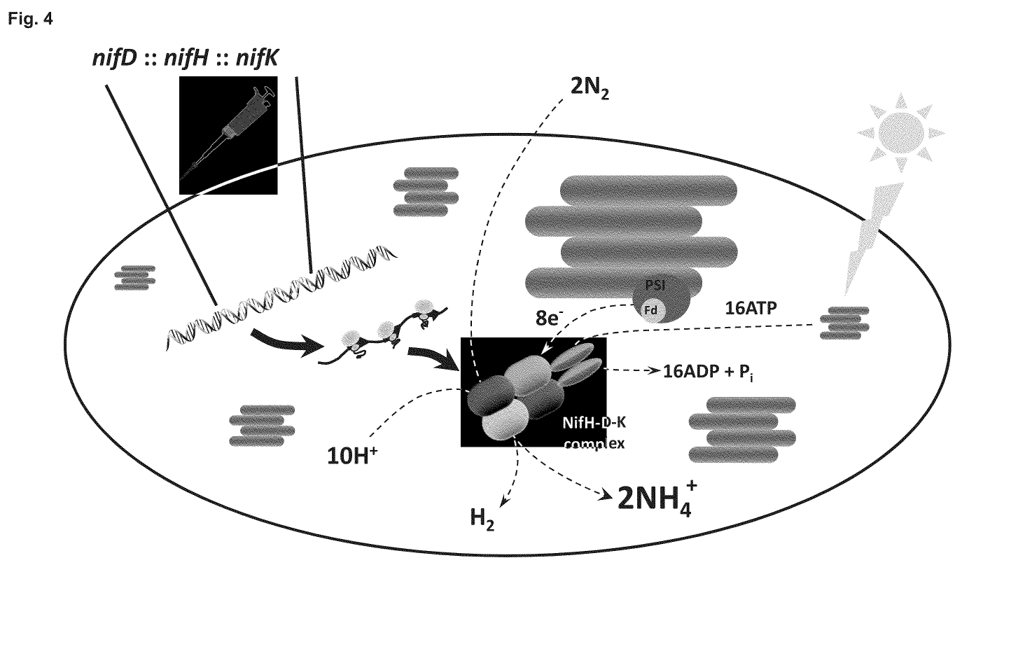

In some embodiments, in accordance with the present disclosure, a target plant is genetically transformed with the DNA containing the gene sequences from cyanobacteria having Nif-H, Nif-D and/or Nif-K (SEQ. ID. NOs. 1-3 and/or 29-40). In the alternative, the target plant can be genetically transformed with similar genes from other bacteria under specific conditions to be expressed in the plant plastid (promoter and starting translation sequences, as well as homologous recombinant sequences). This is not limited to the plastid (chloroplast). FIG. 3 and FIG. 4 show a plasmid used in the plastid transformation.

The target plant, which refers to the plant cell or tissue that will have the new genes or gene introduced to it, will be genetically transformed with the “new” DNA containing the gene sequencing of the Nif-H, Nif-D and Nif-K from cyanobacteria (i.e., Cyanobacterium Anabaena PCC 7120 (SEQ. ID. Nos. 1-2) (Or other Cyanobacteria (SEQ I.D. NOs. 29-40), or other bacteria (only under specific conditions when there is an accumulation of the Nif proteins, “enzyme”, in the plastid). A plant transformed to have these genetic features has the ability to function as a self or auto nitrogen fixation machine. Self or auto nitrogen fixation refers to the cell (plant cell), the plant, or parts of plants like roots or leaves that can fix nitrogen into ammonia. This is not by symbiosis with other organisms like the rhizobium. An alternative sequence may be used for the target plant. For example, the Nif-D can be replaced by the coding sequence of each of the other Nif-D, and the same role applied to Nif-K and Nif-H, respectively.

Once the plant is genetically modified as described herein, it is capable of self biological nitrogen fixation and can be exploited for the production of nitrogen fixation in plants. The newly engineered plant will produce nitrogen in a form available for the plant's use or animal use.

Any plant can use this invention like the tomato plant (Lycopersicum sp.) or the tobacco plant (Nicotana tabacom). These can be used as a commercial product, as an agricultural dissemination, as agricultural root-stock (using grafting technique) or as a model plant organism for plant research.

In some embodiments, the source of the Nif genes (referred to as coding sequences or mRNA, RNA or DNA) can be bacterial carriers. The Nif genes are found in unique photosynthetic bacteria like cyanobacterium. Expression of the Nif-H gene in a plant cell, in a plastid or a chloroplast allows the Nif-H protein time to assemble into an active enzyme that can functionally serve as the obligate electron donor to the Nif D-K complex. This expression allows the Nif-H to function as nitrogenase reductase. In some embodiments, the Nif genes are created synthetically and are not found in nature. See, for example, SEQ. ID. NOs. 29-40.

The expression of the Nif-D gene and the Nif-K gene in a plant cell or in a chloroplast allows for the accumulation of the Nif-D-K proteins. The above gene expression within the plant cell and/or plastid is known as a Nitrogenase Mo—Fe protein and functions as sub unit nitrogenase. The combination of the above will produce a plant that is capable of fixing/providing some or all of its nitrogen needs. Plants that can fix nitrogen will be more robust because it will not need to be dependent on symbiosis bacteria, nor will it be dependent on chemical or organic sources of nitrogen. The use of Nif-H, Nif-D and Nif-K genes in plant cells mimics the function of the bacteria or prokaryotic core pathway in nitrogen fixation.

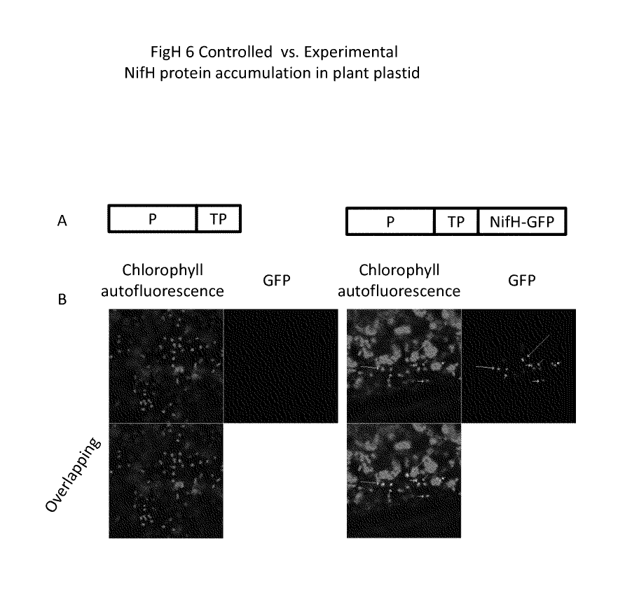

Plant cells that accumulate the Nif protein in any combination of Nif-H, Nif-K and Nif-D will fix nitrogen into one of the consumable forms (ammonia (NH₃) and then into ammonium (NH₄). The expression from the nuclei of one or all of the above can target it to the plant plastid to allow for a better control on the time and accumulation of the function Nif complex (Nif-H and Nif-D-K), as shown FIG. 6.

A method for producing a transgenic plant with a modified self/auto nitrogen fixating profile is provided wherein the transgenic plant comprises in its genome or in its plastid, genome a combination of Nif-H, Nif-D and Nif-K genes so as to allow the plant to mimic bacteria or prokaryotic core pathway for nitrogen fixation. The method comprises transforming a host plant by inserting recombinant Nif-H, Nif-D and Nif-K genes into the genome of the host plant wherein the Nif-H, Nif-D and Nif-K genes are respectively operably linked to a promoter sequence, a terminator sequence and optionally to a DNA sequence encoding a targeting signal or a transit peptide, all active in said host plant. In some embodiments, the recombinant Nif-H, Nif-D and Nif-K genes are included in SEQ ID. NO. 1. In some embodiments, the recombinant Nif-H, Nif-D and Nif-K genes are included in SEQ ID. NO. 2. In some embodiments, the recombinant Nif-H, Nif-D and Nif-K genes are included in SEQ ID. NO. 3. As would be appreciated by one of ordinary skill in the art, because SEQ ID. NOs. 1 and 2 each include the promoter identified by SEQ ID. NO. 4A, it is not necessary to transform an additional promoter into the host plant when using either SEQ. ID. NO. 1 or SEQ. ID. NO. 2. Likewise, because SEQ ID. NO. 3 includes the promoter identified by SEQ ID. NO. 4B, it is not necessary to transform an additional promoter into the host plant when using SEQ. ID. NO. 3. Alternatively, the sequences can be transformed into the nucleus by adding transit peptides and the product will acculmenate in the plastid.

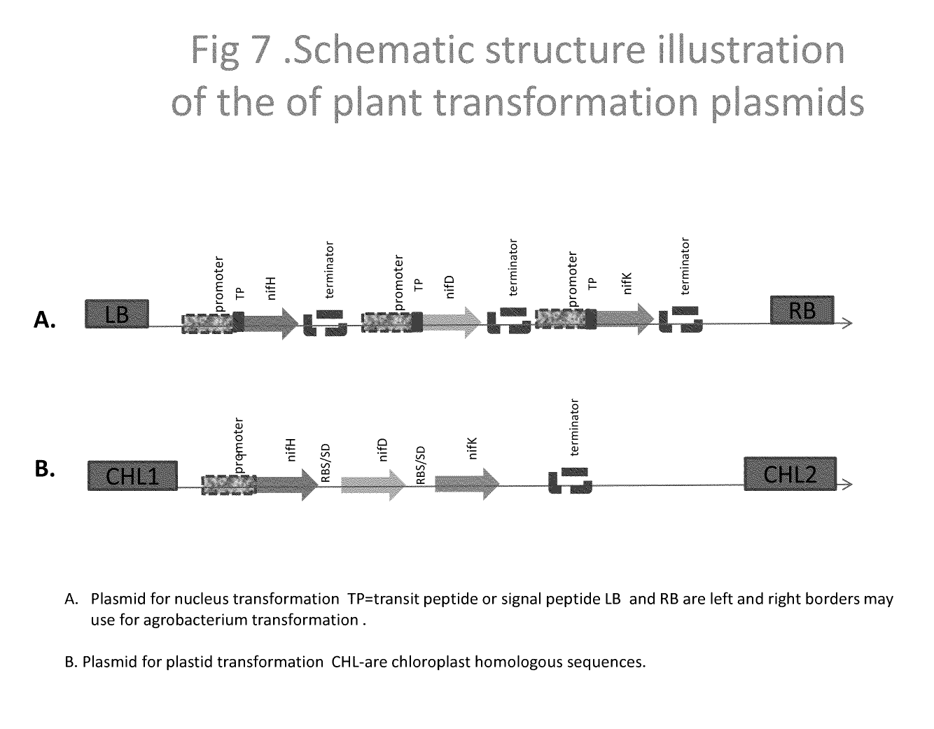

In some embodiments, a method is provided in accordance with the principles of the present disclosure that produces a transgenic plant with a modified self/auto nitrogen fixating profile comprising in its genome (plastid genome) a combination of Nif-H, Nif-D and Nif-K genes so as to allow the plant to mimic bacteria or prokaryotic core pathway for nitrogen fixation, wherein the method comprises the steps of: contacting one or more plant cells with a recombinant nucleic acid sequence encoding Nif-H, Nif-D and Nif-K genes operatively linked to a promoter, such as, for example, a rrn16 promoter; regenerating one or more plants from the plant cells; and selecting one or more plants cultivated from the plant cells, wherein the selected plants each exhibit enhanced nitrogen fixation, wherein the selected plants each comprise the recombinant nucleic acid sequence encoding the recombinant nucleic acid sequence operatively linked to the rrn16 promoter. This method is shown in portion B of FIG. 7. In one embodiment, the recombinant nucleic acid sequence encoding Nif-H, Nif-D and Nif-K genes is operatively linked to a single promoter, such as, for example, a rrn16 promoter. In some embodiments, the recombinant nucleic acid sequence includes at least a portion of at least one of SEQ. ID. NOs. 1-3 and SEQ. ID. NO. 49.

In some embodiments, a method is provided in accordance with the principles of the present disclosure that produces a transgenic plant with a modified self/auto nitrogen fixating profile comprising in its genome a combination of Nif-H, Nif-D and Nif-K genes so as to allow the plant to mimic bacteria or prokaryotic core pathway for nitrogen fixation, wherein the method comprises the steps of: contacting one or more plant cells with a recombinant nucleic acid sequence encoding a NifH gene operatively linked to a promoter; contacting the plant cells with a recombinant nucleic acid sequence encoding a Nif-D gene operatively linked to a promoter; contacting the plant cells with a recombinant nucleic acid sequence encoding a Nif-K gene operatively linked to a promoter; regenerating one or more plants from the plant cells; and selecting one or more plants cultivated from the plant cells, wherein the selected plants each exhibit enhanced nitrogen fixation, wherein the selected plants each comprise the recombinant nucleic acid sequence encoding the Nif-H gene, the Nif-D gene and the Nif-K gene, wherein the nucleic acid sequences encoding the Nif-H, Nif-D and Nif-K genes are each operatively linked to a promoter. This method is shown in portion A of FIG. 7.

In some embodiments, the recombinant nucleic acid sequences encoding the Nif genes (Nif-H, Nif-D, Nif-K) include at least three of a group consisting essentially of SEQ. ID. NOs. 29-40. In some embodiments, the recombinant nucleic acid sequence encoding the Nif-H gene is selected from a group consisting essentially of SEQ. ID. NOs. 29, 32, 35 and 38. In some embodiments, the recombinant nucleic acid sequence encoding the Nif-D gene is selected from a group consisting essentially of SEQ. ID. NOs. 30, 33, 36 and 39. In some embodiments, the recombinant nucleic acid sequence encoding the Nif-K gene is selected from a group consisting essentially of SEQ. ID. NOs. 31, 34, 37 and 40. In some embodiments, the promoter is selected from a group consisting essentially of SEQ. ID. NOs. 41-45. In some embodiments, the nucleic acid sequences encoding the Nif-H, Nif-D and Nif-K genes, respectively, are present in a single organism found in nature. In some embodiments, the nucleic acid sequences encoding the Nif-H, Nif-D and Nif-K genes, respectively, are present in a different organism found in nature. In some embodiments, each of the nucleic acid sequences encoding the Nif-H, Nif-D and Nif-K genes are synthetic and are not found in nature. In some embodiments, at least one of the nucleic acid sequences encoding the one of the Nif-H, Nif-D and Nif-K genes is/are found in nature and at least one of the nucleic acid sequences encoding one of the Nif-H, Nif-D and Nif-K genes is/are synthetic and is/are not found in nature. In some embodiments each of the nucleic acid sequences encoding one of the Nif-H, Nif-D and Nif-K genes includes a terminator. In some embodiments, at least one of the Nif-H, Nif-D and Nif-K genes is linked to a different promoter than another of the Nif-H, Nif-D and Nif-K genes. In some embodiments, the promoter comprises SEQ. ID. NO. 4 and/or SEQ. ID. NO. 28. In some embodiments, each of the Nif-H, Nif-D and Nif-K genes is linked to the same type of promoter, such as, for example, a rrn116 promoter.

In some embodiments, the methods discussed in any one of the preceding paragraphs include at least one of the following steps: identifying a target organism; determining whether the target organism is better suited for nucleaus transformation or plastid transformation; identifying a protein sequence of a donor organism that includes Nif genes; reverse translating the protein sequence into DNA using a universal genetic codon; optimizing a coding DNA sequence (CDS) to the target organism (i.e. tomato plant); synthesizing a synthetic CDS; adding a promoter terminator; transforming the CDS into a plant for stable or transit transformation; and assaying for nitroganese activity by one or more of the following assays: ARA, 15N stabile isotope incorporation or the ability to grow on nitrogen depletion media. In some embodiments, all of the Nif-H, Nif-D and Nif-K genes are linked to the same promoter as one operon, such as, for example, a rrn116 promoter.

In some embodiments the donor organism comprises single cell cyanobacteria that are photosynthetic and have nitrogen fixation ability. In some embodiments, the Nif genes of the donor organism are identified by blastN or by review of literature. In some embodiments, the Nif genes identified include Nif-H, Nif-D and Nif-K genes. In some embodiments, the coding DNA sequence for the target organism is optimized by using ad hoc codon usage of Rubisco so as to not neglect less frequently used codons, unlike common codon usage optimizers that use only the most frequent codon and neglect all others (Seq ID. NOs. 35-37). In some embodiments, the coding DNA sequence for the target organism is optimized by using standard codon optimyzer and most usage of Rubisco SEQ ID. NOs. 29-36. In some embodiments, the coding DNA sequence for the target organism is optimized by using total chloroplast known usage codon SEQ NOs. 38-40.

Provided in accordance with the principles of the present disclosure is a transgenic plant with a modified nitrogen fixation producing profile comprising in its genome recombinant Nif-H, Nif-D and Nif-K genes operably linked to a promoter sequence and a terminator sequence.

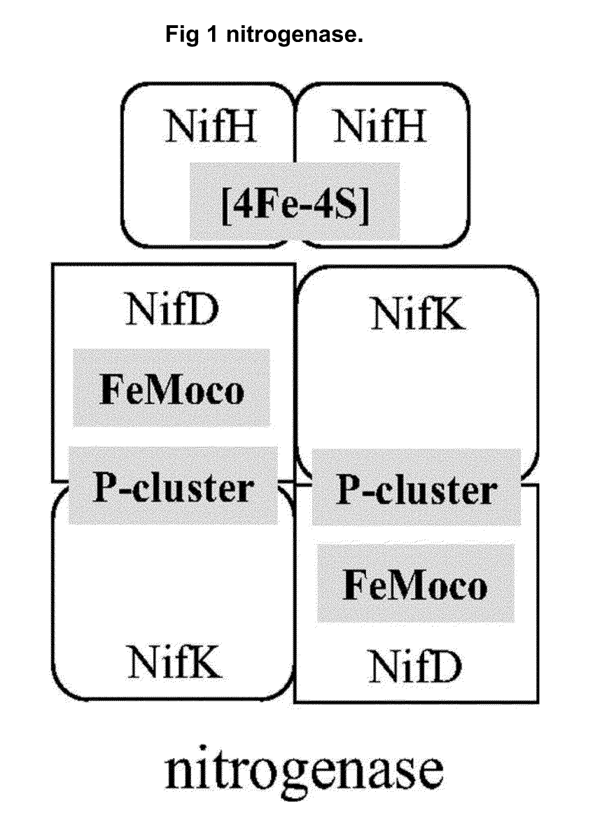

FIG. 1 shows the schematic structure of the nitrogenase complex. It is formed by two Nif-H proteins with four atoms of iron as the cofactor (homodimer). The Nif-K proteins form a hetrodimer with two subunits of Nif-D and two subunits of Nif-K with molybdenum as the cofactor.

In another aspect of the present disclosure self or autonomous biological nitrogen fixation (sBNF) crop plants are provided. For example, tomato plants (Lycopersicon sp.) can carry an expressed gene for the nitrogenase reductase enzyme (Nif-H and NifD-K) so that it can mimic the bacterial pathway. The nucleic acid sequence for the nitrogenase reductase enzyme (Nif-H and NifD-K) are provided as SEQ. ID. Nos. 1 and 2, as shown in FIG. 6, SEQ. ID. NO. 29.

In another embodiment in accordance with the principles of the disclosure, self or autonomous BNF in crop plants and/or in any other plant with optimization of the Nif genes coding sequence in the target plant organelle (plastid) using codons to replace the original bacterial sequence are provided. For example, tomato plants (Lycopersicon sp.) can carry an expressed gene for the nitrogenase reductase enzyme namely, Nif-H and NifD-K so that it can mimic the bacterial pathway.

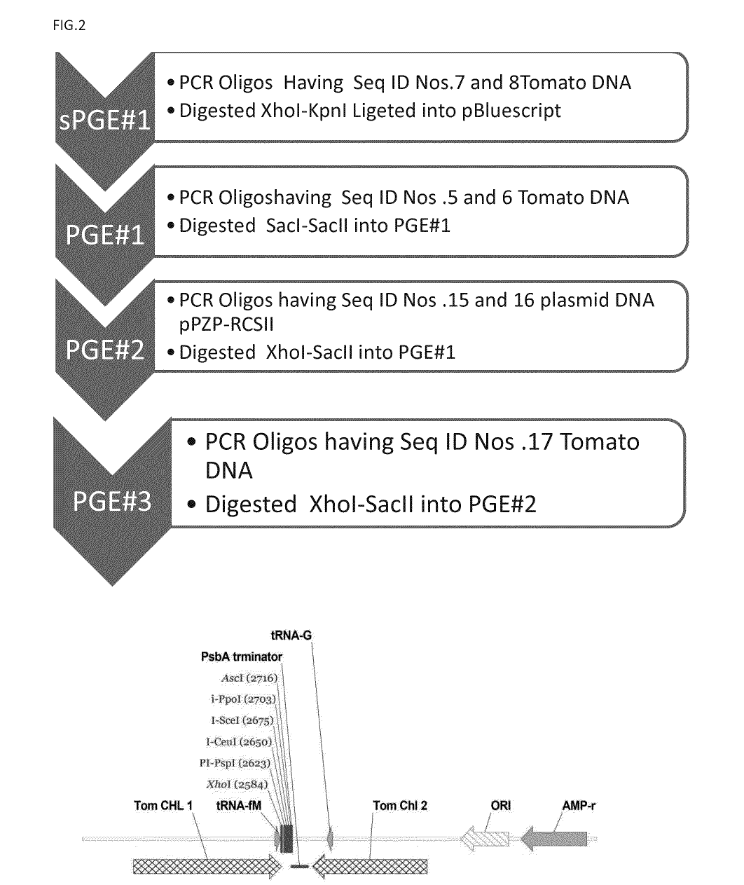

FIGS. 2 and 3 show a schematic representation of the steps used to produce the plasmid for plasmid for tomato chloroplast transformation. (PGE 003): A Polymerase Chain Reaction (PCR) was used to amplify DNA by specific primers or oligos (Table 1) flow by digestion with restrictions to enzyme and ligation into the plasmid upper part. After 4 steps the product contains 2 chloroplast homologues sites known as tRNA-FM and t-RNA-G, with several unique cloning sites flowing by the terminator of PsbA.

FIG. 3 shows the schematic representation of the steps for producing the plasmid for tomato chloroplast transformation and the expression of the Nif-H, Nif-D, and Nif-K genes necessary for tomato plant transformation. A polymerase chain reaction (PCR) was used to amplify DNA by specific primers or oligos (Table 1) to be followed by digestion with restriction enzymes and ligation into a plasmid. After one or two steps, the plasmid contains the Nif genes and coding sequences are made. The three genes, a Nif gene, a reporter gene, and a selection marker such as, for example, aadA are cloned as one cluster by using a cutter such as, for example, a restriction enzyme.

FIG. 4 shows the Nif-H, Nif-D and Nif-K genes as a single operon inserted into a chloroplast of a C. reinhardtii or other chloroplasts. The chloroplast contains prokaryotic gene expression systems, allowing the operon to be expressed. Electrons and ATP donated by the photosynthesis allow the nitrogenase enzyme to act, resulting in production of NH₄. Algal ferredoxin (Fd) mediates electron transfer from the photosystem I (PSI) to the Nif-H/Fe-protein of the nitrogenase. The present disclosure suggests that the Nif complex is less sensitive to oxygen due to its original source and that the Fe-protein (Nif-H) is able to transfer forward the electrons to nif-K to aid in the auto/self fixation of nitrogen.

It is envisioned that the principles of the present disclosure may be applied to any plants, such as, for example, rice, corn potatoes, squash melons, tobacco, cotton Arabidopsis, and trees like apples, cherries, walnuts, and also green algae, as well as other plants. For example, to apply the technology to chlamydomonas (green algae) the coding sequence was optimized by synthetic gene synthesis having SEQ. ID. No. 3.

Another embodiment of this disclosure provides a method of using transgenic plants (genetically modified organisms (CMOs)) to reduce the need or demand for nitrogen fertilization. Another embodiment of this disclosure provides a method of using transgenic plants (genetically modified organisms (CMOs)) to replace the nitrogen fertilization (e.g. the GMOs can be used as a plant fertilizer).

The Examples provided below describe the genetic manipulation of a plants, i.e. tomato plants, to have the ability to fix its own nitrogen are achieved.

### Example

### Creating Plasmid for Tomato Plastid Transformation

In the present disclosure, prokaryotic or bacterial nitrogenase and BNF are exploited for the production of ammonia and/or ammonium in plants. These transgenic plants have the ability to produce all or part of its nitrogen demands once transformed with plasmids having the code for BNF. Crops suitable for human consumption like tomatoes, rice or wheat, and suitable as horticultural plants like flowers, grass and trees, including other plants and algae can also be transformed to auto/self fix nitrogen. Since these plasmids do not exist in nature, synthetic biology is used to create the artificial plasmid that enables cells/plants to auto/self fix nitrogen. The artificial plasmid contains a modification of the CDS and the controls sequences as well to carry out novel tasks of expression and accumulation of nitrogenase in plant, for example.

### Example

### Isolation of Genetic Material

Plant DNA Isolation: Young tomato plants at 3-4 weeks were used to isolate genomic (nuclease) and plastid DNA using a DNeasy Plant Mini Kit (Qiagene Germany). The pure DNA was used to complete PCR amplification for the DNA sequence of interest.

The Tomato DNA was amplified by specific PCR reactions using SEQ. ID. Nos. 5, 6, 7, and 8 (See table 1). This resulted in tomato recombination sites (SEQ. ID. Nos. 5, 6, 7, and 8 (See table 1) and termination of PsbA signals (SEQ. ID. Nos. 17 and 18). All three PCR products are from (tomato) plastid DNA.

PsbA is the gene for the D1 protein (also known as PsbA), which forms the reaction core of the Photosystem II Reaction Center. It is well known as a constitutively expressed gene.

The PCR amplified DNA was then used for digestion by restriction enzymes to create cohesive ends or sticky ends on the SacI-SacII, XhoI-KpnI, and BamHI-AscI that attached to each of the PCR products SEQ. ID. Nos. 5, 6, 7, 8, 17 and 18 respectively.

Cohesive ends or sticky ends are terms used when the restriction enzyme creates either a 3′ or 5′ overhang. These overhangs are in most cases palindromic (symmetric). Each cohesive end will be ligated exclusively to its complimentary sequence. Ligated or ligation refers to the reaction of covalent linking of two ends of DNA molecules that is usually preformed by an enzyme like T4-DNA ligase, but is not limited to DNA or DNA ligase, which “glues” the DNA fragments together.

The PCR restricted DNA was cloned into pBluescript plasmid in a three state reaction as shown in FIG. 2, by incubation with T4-DNA ligase enzyme (NEB.USA), completed in the recommended factory conditions.

To add unique restrictions for multiple cloning sites (MCS) that are non-palindromic, the MCS of pPZP-RCSII was amplified by PCR and cloned into the XhoI-SacI site as shown in FIG. 2 and FIG. 7.

The new plasmid named PGE 003 consists of the AMP-R or BLA gene and the ORI of the pBluescript and the tomato chloroplast site for homologous recombination (Tom Chl1 and 2 in FIG. 2), and the PsbA terminator from the tomato, with the MCS artificial DNA sequences of pPZP-RCSII.

The PGE 003 plasmid produced using this procedure has unique Multiple Cloning Site MCS-unique DNA with restriction sites allow to use restriction enzyme for cloning consisting of the flowing restriction enzymes: XhoI, Pi-PspI, I-CeuI, I-SceI, I-PpoI, and AscI allowing it to clone genes of interest in order and orientation. Since the Pi-PspI, I-CeuI, I-SceI, and I-PpoI, are not palindromic, the sticky end is not symmetrical. The ligation will therefore take place only in one direction.

### Example

### Cloning of Nif Genes and Expression

Tomato DNA was amplified by specific PCR reactions using oligos having SEQ. ID. Nos. 19 AND 20 and cloned into an AgeI-BamHI site of pSAT6-MCS creating PGE 006. This is a temporary plasmid with DNA of interest in-between the Pi-PspI sites. By this cloning step, the promoter of the rrn16 gene from the tomato DNA was cloned.

The pSAT-vectors are serials of vectors (plasmid) that use homing endonucleases. Homing endonucleases are restriction enzymes that usually have a long recognition site. This characteristic makes them “rare cutters” because the frequency of their cutting in any randomly chosen DNA is rare. For example, pSAT4 has 2 repeats of the site for I-SceI AGTTACGCTAGGGATAACAGGGTAATATAG SEQ. ID. No. 25 (available as Genbank SEQ. ID. No. DQ005466) and these are 30 bases compared to 6-8 of a regular restriction enzyme.

PCR on Cyanobacteria ATCC7120 DNA were amplified by a specific PCR reaction using oligos having SEQ. ID. Nos. 9 and 10 (see table 1) and cloned into (restriction site) BgIII-NotI sites of (Plasmid for plant Genetic Engineering)(PGE) 006 creating PGE 007 which is a plasmid with the Nif-H gene under the control of a rrn16 promoter.

The rrn16-promoter refers to the 5′ DNA sequence of the plastid gene for ribosomal RNA 16S that is a well-known expression promoter in plastids and is used in the present disclosure, however other inducible promoters are also possible. That is, the promoter is selected to be inducible under any condition where it would be desirable to cause the plant to have auto/self nitrogen production and/or enhanced nitrogen uptake, assimilation or use capabilities. For example, suitable promoters may include, but are not limited to, those which are induced by application of sources of nitrogen, stress inducible, wound inducible or induced by application of other chemicals. Transgenic plants containing the genetic construct of the present disclosure exhibit enhanced agronomic characteristics over control plants. The particular agronomic characteristic which is enhanced usually depends on the nature of the promoter and can include enhanced stress tolerance and/or more efficient nitrogen uptake, storage or metabolism allowing the plants of the present invention to be cultivated with little to no nitrogen fertilizer input and in nitrogen starved conditions or allowing faster growth, greater vegetative and/or reproductive yield under normal growing conditions.

PCR on Cyanobacteria ATCC7120 DNA was amplified by specific PCR reactions using oligos having SEQ. ID. Nos. 11 and 12 (see table 1) and cloned into AgeI-XhoI sites of pSAT5-MCS creating PGE 008, a plasmid with the Nif-D gene.

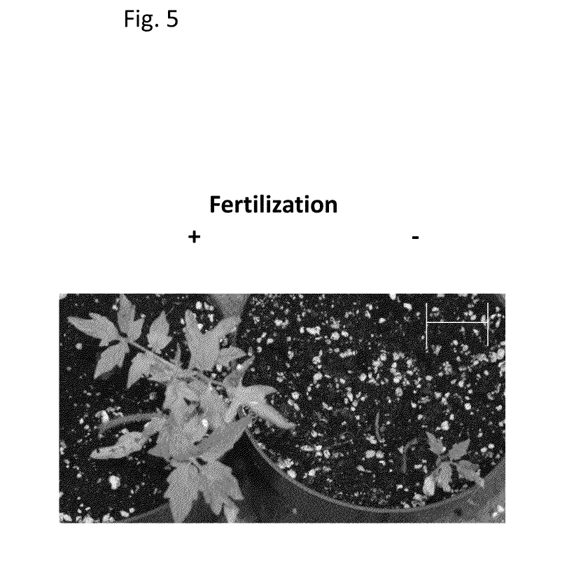

PCR on Cyanobacteria ATCC7120 DNA was amplified by specific PCR reactions using oligos having SEQ. ID. Nos. 13 and 14 (see table 1) and cloned into XhoI-NotI sites of PGE 008 creating plasmid with Nif-D and Nif-K genes (FIG. 5 upper center). PCR on pPZP-RCSII DNA was amplified by a specific PCR reaction using oligos having SEQ. ID. Nos. 23 and 24 (see table 1) and was cloned into AgeI-XhoI sites of pSAT4, that created PGE 005 plasmid with aadA genes (FIG. 4 upper right). The aadA is the reporter gene for the plant transformation. This gene encodes the enzyme aminoglycoside 3′ adenylyltransferase that inactivates spectinomycin and streptomycin by adenylation, and prevents binding to chloroplast ribosomes, which allows the plant to grow on media that contains the antibiotic. Alternatively after the basic design on the computer, the entire nucleic acids sequence is submitted into commercial software and produce the synthetic plasmid, by synthesis overlapping oligos (oligos are usually short DNA sequences) sequences with overlapping and consecutive bases connected to each other to create the entire sequence. Using this technology allows to create new sequences expressed in a cell-specific optimal. The plasmids PGE having SEQ. ID. NOs. 9, 11, and 13 were digested by PI-PspI, I-CeuI and I-SceI respectively, and donated the genes of interest, e.g. Nif-H, Nif-D, Nif-K and aadA into the same site on having SEQ. ID. NO. 7 where the new plasmid having SEQ. ID. NO. 15 was used for the plastid.

PGE having SEQ. ID. NO. 2 was then bound to gold particles and bombarded into the leaf tissue of a tomato plant.

Tomato chloroplast transformation allows the incorporation of foreign DNA based on homologous recombination between known sites of the plant plastid DNA (trnfM and tmG) in the tomato plant. In general, a few hundred base pairs (BPs) of each side enables it to transfer a few thousand new BP in between (Nif genes) (see FIG. 5). The use of plasmid DNA carries these genes of interest which: 1) maintains the DNA in bacteria like E. coli; 2) promotes mutagenesis of the CDS; and 3) binds the plasmid DNA to gold particles and bombards it to the tomato plant's leaf tissue. Adding a selection marker like aadA and selecting the transfected tissue on plant media containing streptomycin or spectinomycin antibiotics will result in killing the cells that do not contain the “marker”. After a few weeks, cells that survive will contain the transgenic plastid (chloroplast) homoplastid suggesting that all of the chloroplasts are identical and contain the new genes, which can be proven by a simple site-specific PCR test. The plant's leaf will develop into a new plant (regeneration) depending on the hormone's concentration in the media.

The transgenic plant will express the Nif operon, of Nif-H Nif-D and Nif-K or any two out of these three. The term “operon” refers to a few genes organized in DNA sequence order and transcripted together. The gene referred to is the DNA sequence or a partial of DNA sequence that can be transcripted to a RNA molecule and/or translated to a protein or peptide.

After bombardment of the plasmid DNA to the leaf, heterologous recombination takes place. As a normal part of the plant's cell life cycle the two molecules (plasmid DNA and plastid DNA) will replace parts of the plant's DNA according to the similarity between the recombination sites. As a result, an extra piece of the plasmid DNA will be introduced into the plastid DNA.

As the plant tissue is exposed to antibiotics, only plastids that can avoid being harmed by the antibiotic (are resistant to the antibiotic) will survive, and eventually after a few generations, only plastids that are transgenic will be present in each cell. At this point the plant, cell, or tissue involved will be considered a homoplamic.

Sterile tomato plants (L. esculentum var. IAC-Santa Clara) were raised in Magenta boxes (double boxes with a connector element) from surface-sterilized seeds germinated on an MS medium. For biolistic bombardment, young leaves were harvested from three to four week-old plants (approximately 15 cm high) produced from outgrowing axillary meristems in stem cuttings. Homoplasmic transplastomic plants and wild-type control plants were transferred to the soil and grown to maturity in a phytochamber (16 hours in light, 8 hours dark, at 24° C.). Control plants were grown under identical conditions.

Plastid transformation of the tomato plant was achieved by biolistic bombardment of young sterile tomato leaves with plasmid DNA-coated gold particles of 0.6 μm diameter using the DuPont PDS1000He biolistic gun and 1,100 p.s.i. rupture disks (BioRad Laboratories, Hercules, Calif.). Bombarded leaf samples were cut into small pieces (3×3 mm), transferred to a RMOP medium containing spectinomycin (300-500 mg/L), and incubated under dim light (25 μE; 16 h light, 8 h dark) for three to four months. Primary spectinomycin-resistant lines were identified as yellow or pale green growing calli. Callus pieces were transferred to the same medium for further propagation and isolation of homoplasmic transplastomic tissue. For plant regeneration, homoplasmic callus tissue was transferred onto the surface of agar-solidified MS medium containing 0.2 mg/L IAA and 3 mg/L BAP. Alternatively, shoot induction was obtained with the same medium but 2 mg/L zeatin instead of BAP. For rooting, regenerated shoots were transferred into boxes containing phytohormone-free MS medium. RNOP medium is MS supported with growth hormones: NAA 0.1 mg/L, BAP 1 mg/L, and Vitamins Thiamine 1 mg/L, Myo-inositol 100 mg/l and 30 g of Sucrose as a carbon source.

Alternative genes or proteins may be used for this invention for example genes set forth in Table 2, since microorganisms like cyanobacteria have microbial diversity. Since some genes are not identical but similar and function almost identically, different sources for the Nif-D, Nif-H, Nif-K and CDS can be used.

FIG. 5 shows the development of tomato plants at three weeks post germinating. The left pot was supplied with a single dose of fertilizer at the time of planting and in the right pot no fertilizer was added to the plant. They were kept together under normal field conditions and the size marker on the right is 1 inch.

FIG. 6 shows targeting and accumulation of Nif-H in plant cells. In particular, (A) a non-heterocystous cyanobacteria NifH from Leptolyngbya nodulosa served as a template for the synthesis of Nif-H-GFP. For the transit peptide 210 bases were added encoding the tomato Rubisco transit peptide at the 5′ end. Empty vector was used as control. (B) shows chloroplast autofluorescence and GFP photographed by confocal microscopy. Overlap (merge) images demonstrate that TP-NifH-GFP can accumulate at the leaf chloroplast and cannot be detected at other parts and organelles of the cell.

Another example that can be used is the single cell algae Chlamidomonas reinhardtii that can be used as model for biofuel or biodiesel production or as a green fertilizer, it is genetically transformed with DNA containing the gene sequences of the NifH and NifD and NifK from cyanobacteria SEQ Nos. 1-3 (or other nitrogen-fixing bacteria) with specific elements allowing expression in the plastid, i.e., promoter and translation initiation sequences as well as homologous recombination sequences.

The resulting genetically modified C. reinhardtii will carry and express the genes for the nitrogenase reductase enzyme, mimicking the bacterial pathway for nitrogen fixation. To exploit BNF for nitrogen fixation in plants through genetically engineered C. reinhardtii produces nitrogen in an organic form available for plant or animal consumption. Many algae can be used with this technology with only minor adjustments for commercial products, producing faster growth and environmentally friendly results that cost 15-35% less than traditional fertilization. The source for the Nif genes when transforming algae is photosynthetic cyanobacterium. Using these Nif genes ensures that the transformed algae will be able to utilize the enzyme for its metabolism.

The present disclosure describes a new plasmid that allows expression of the Nif genes as one operon. The unique configuration avoids complications of previous experiments, which resulted in expression of both individual and separate genes.

Additional sources of Nif genes and isolation of Nif genes will be completed as bacterial genome sequences become public knowledge. The Nif genes of cyanobacteria available by amplifying the specific target gene. For example, in the genomic sequence of Nostoc sp. FCC 7120 accession number BA000019.2 (NCBI), Nif-H can easily be amplified by PCR using the first and last 24 bases of the gene, starting at ATGACTGACGAAAACATTAGACAG (SEQ. ID, No. 3) and ending at ATGACTGACGAAAACATTAGACAGA (SEQ. ID. No. 25). Adding restriction sites to the beginning of each primer allows one to clone the PCR product in a specific site that is not found within the coding sequence, such as the XhoI site for the first primer: CTCGAG-ATGACTGACGAAAACATTAGACAG (SEQ. ID. NO. 26 and the XbaI site for the second primer TCTAGA-ATGACTGACGAAAACATTAGACAGA (SEQ. ID, No. 27). This enables one to clone the NifH gene into the XhoI-XbaI sites. Ligation of the PCR product at the size of 900 bp into plasmid, such as pBluescript, followed by the selection on an ampicillin-containing LB-agar plate allows verification of the sequences. The same strategy will apply to cloning the Nif-D and Nif-K genes.

### Example

### Plastid Transformation

C. reinhardtii chloroplast transformation allowing incorporating foreign DNA based on homologous recombination between known sites of the plant plastid DNA like (trnfM and tmG) is shown in FIG. 2-3 and uses one of SEQ ID. NOs. 1-3.

In general, a few hundred base pairs of each side is enabled to transfer a few thousand new DNA base pair (BP) in between (Nif genes). The plasmid DNA carrying these genes: 1) maintains the DNA in bacteria like E. coli; 2) allows sequence and/or mutagenesis of the coding DNA sequences (CDS); and 3) is available for genetic transformation.

The genetic transformation (inserting a plasmid into a pastid—see FIG. 7B) includes the following steps: 1) adsorbing DNA, such as, for example, a nucleic acid sequence encoding SEQ. ID. NO. 1, SEQ. ID. NO. 2 or SEQ. ID. NO. 3 onto gold particles and bombarding the DNA onto a C. reinhardtii cell; 2) adding a selection marker, such as, for example aadA, and selecting the transformed cells on algal growth media containing an antibiotic, such as, for example, spectinomycin so as to eliminate the cells that do not contain the transgene; 3) storing the cells that contain the transgenic plastids; and 4) verifying the sequence using simple site-specific PCR.

Once modified, C. reinhardtii will be tested for nitrogen fixation by acetylene reduction, for example. This process measures the amount of acetylene, which is source of nitrogen, using a gas chromatographer so as to provide direct proof of the Nif enzyme activity. Growing the transformed bacteria and comparing it to the wild type confirms that the transformed genes are active, thus allowing C. reinhardtii to fix nitrogen.

### Example

### Nucleus Transformation (See FIG. 7A)

Preparation of Tobacco Plants:

Tobacco plants are grown for 3 to 4 weeks post-germination in Majenta boxes, on MS medium at 22° C. in a long day growth chamber.

Preparation of Agrobacterium Culture:

An agrobacterium culture is prepared by growing seed culture overnight (28° C., 250 rpm, from a freshly re-streaked LB plate), in 3 ml LB+antibiotic (e.g., rifampin* 10 mg/l and spectinomycin 100 mg/ml)+plasmid Seq # PGE0048 or PGE0066 or PGE0088 or PGE0148. 1 or 2 ml is diluted into 50 ml LB without antibiotic and grown for 2 to 6 hours at 28° C. until OD600=0.6 to 1.0.

Transformation Procedure:

The bacterial culture is poured into a sterile Petri dish. Leaves of the tobacco plants are cut into smaller pieces, such as, for example, discs or squares so that the smaller pieces are generally about 2 cm×2 cm pieces.

The pieces of the tobacco plants are added to the bacterial culture in the Petri dish. The Petri dish is incubated for about 20 minutes at 22-25° C. The pieces of the tobacco plants are dried on sterile filter paper to eliminate any excess liquid. The dried pieces of the tobacco plants are placed onto a MR plate (if possible, adaxial side down). The plate is sealed with parafilm.

Alternatively, one or more leaves of the tobacco plants may be injected with OD 0.1 of bacterial suspension. The injected leaves are then given 48-72 hours to recover. The injected leaves are analyzed for transit expression of the Nif genes. After 48-72 hours, the plants are closed in chamber with 15N stable isotope for 96 hours and then were analyzed for incoupration of 15N into the plant amino acid and or other molecol by mass spectrometry.

Co-Cultivation:

Incubate the leaves/pieces for 3 days in a growth chamber at 22° C. (long days). After 3 days, the leaves/pieces was/were washed by rinsing in sterile water with Timentin 100 mg/l in the water. The leaves/pieces are put onto MRTK, with the leaves/pieces being spaced apart from one another to reduce the risk of agrobacterium re-growth). The leaves/pieces are placed in a growth chamber for about 2-3 weeks at 22° C. (long days). Calli will appear on the edges of the leaves/pieces. When small buds appear on the calli, replate them on MSTK plates while preferably removing surrounding callus and leaf tissue without damaging the plantlet. After about 3 weeks (sometimes more) small plants will develop and roots will appear, then small plants can be replated on MST (in majenta boxes, cut the roots before replating). Then replate on MS majenta boxes every 3-4 weeks.

Medium Composition

MR=MS medium (1 L), MS powder 4.4 g, Sucrose 30 g, MES 0.5 g pH 5.8 Agar 8.0 g. After autoclave, add BAP 1 ml of stock solution (1 mg/ml) and NAA 0.1 ml of stock solution (1 mg/ml).

MRTK=Same as MR, but after autoclave, add Timentin 300 mg/L final (from a filter sterilized stock solution 300 mg/ml) and Kanamycin 50 mg/L final (from a filter sterilized stock solution 50 mg/ml).

MSTK=Same as MRTK without BAP and NAA and with timentin 300 mg/L final and Kanamycin 30 mg/L final.

MST=Same as MSTK, without Kanamycin, wherein Timentin can be reduced to 100 mg/L, and completely removed in the further replating).

Sequence ID No. 1:
Tomato plastid transformation vector PGE 11, complete sequence
Plasmid DNA 1 . . . 12166
Tomato, Cyanobacteria other sequences; artificial sequences; vectors.

| | |
|---|---|
| DEFINITION | Tomato plastid transformation and cloning vector PGE11 |
| | complete sequence. |
| ACCESSION | PGE 0011 |
| KEYWORDS | Tomato plastid transformation; Plant Nif expression region; |
| SOURCE | Plant Genetic Engineering 11 |
| ORGANISM | Escherichia coli XI1B |
| AUTHORS | Zaltsman Adi |
| TITLE | Plasmid for tomato stable transformation and expression of NifH |
| | and NifD-K cluster, modified plasmid vectors |
| FEATURES | Location/Qualifiers |
| CDS | complement(11181 . . . 12038) |
| | AMP^{R} (BLA) product = “beta-lactamase” |
| | /protein_id = “AAA66380.1 |
| rep_origin | complement(10404 . . . 11018) |
| | ORI-PUC |
| misc_feature | complement(8470 . . . 9959) |
| | Tom\Chl\2—Tomato plastid sequences for homologous |
| | recombination |
| misc_feature | 659 . . . 2582 |
| | Tom\CHL\1—Tomato plastid sequences for homologous |
| | recombination |
| CDS | complement(8660 . . . 8730) |
| | tRNA-G |
| CDS | 2495 . . . 2568 |
| | tRNA-fM |
| terminator | 8205 . . . 8399 |
| | PsbA\trminator |
| promoter | 2644 . . . 2836 |
| | rrn16P |
| CDS | 6926 . . . 7855 |
| | aadA Spc/Strep^{R} |
| CDS | 5316 . . . 6851 |
| | Artificial NifK |
| CDS | 3806 . . . 5296 |
| | Artificial Artificial NifD |
| CDS | 2837 . . . 3721 |
| | Artificial NifH |

| | |
|---|---|
| 1 | ctgaaattgt aagcgttaat attttgttaa aattcgcgtt aaatttttgt taaatcagct |
| 61 | cattttttaa ccaataggcc gaaatcggca aaatccctta taaatcaaaa gaatagaccg |
| 121 | agatagggtt gagtgttgtt ccagtttgga acaagagtcc actattaaag aacgtggact |
| 181 | ccaacgtcaa agggcgaaaa accgtctatc agggcgatgg cccactacgt gaaccatcac |
| 241 | cctaatcaag ttttttgggg tcgaggtgcc gtaaagcact aaatcggaac cctaaaggga |
| 301 | gcccccgatt tagagcttga cggggaaagc cggcgaacgt ggcgagaaag gaagggaaga |
| 361 | aagcgaaagg agcgggcgct agggcgctgg caagtgtagc ggtcacgctg cgcgtaacca |
| 421 | ccacacccgc cgcgcttaat gcgccgctac agggcgcgtc cattcgccat tcaggctgcg |
| 481 | caactgttgg gaagggcgat cggtgcgggc ctcttcgcta ttacgccagc tggcgaaagg |
| 541 | gggatgtgct gcaaggcgat taagttgggt aacgccaggg ttttcccagt cacgacgttg |
| 601 | taaaacgacg gccagtgagc gcgcgtaata cgactcacta tagggcgaat tgggtacccg |
| 661 | attggggcgt ggacataagg gtctttatga cacaatcaac aattcgcttc attttcaatt |
| 721 | aggccttgct ctagcttctt taggggttat tacttctttg gtagctcaac acatgtactc |
| 781 | tttacctgct tatgcattca tagcacaaga ctttactact caagctgcat tatataccca |
| 841 | ccaccaatat atcgcaggat tcatcatgac aggagctttt gctcatggag ctatattttt |
| 901 | cattagagat tacaatccgg agcaaaatga agataatgta ttggcaagaa tgttagatca |
| 961 | taaagaagct atcatatctc atttaagttg ggccagcctc tttctgggat tccataccct |
| 1021 | gggactttat gttcataatg atgtcatgct tgcctttggc actccggaga agcaaatctt |
| 1081 | gattgaaccg atatttgctc aatggataca atccgctcat ggtaaaactt catatgggtt |
| 1141 | cgatgtactt ttatcttcaa cgactggccc agcattcaat gcgggtcgaa gcatctggtt |
| 1201 | gccgggttgg ttaaatgctg ttaatgaaaa tagtaattca ttattcttaa caataggtcc |
| 1261 | tggagacttt ttggttcatc atgctattgc ccttggttta catacaacta cattgatctt |
| 1321 | agtaaaaggt gctttagatg cacgtggttc caagttaatg ccagataaaa aggatttcgg |
| 1381 | ttatagtttt ccgtgcgatg gcccaggacg aggcggtact tgtgatattt cggcatggga |
| 1441 | cgcgttttat ttggcagttt tttggatgtt aaatactatt ggatgggtta ctttttattg |
| 1501 | gcattggaag cacatcacat tatggcaagg taacgtttca cagtttaatg aatcttccac |
| 1561 | ttatttgatg ggctggttaa gggattattt atggttaaac tcttcacaac ttatcaatgg |
| 1621 | atataaccct tttggtatga atagtttatc ggtttgggca tggatgttct tatttggaca |
| 1681 | tcttgtttgg gctactggat ttatgttctt aatttcttgg cgtggatatt ggcaggaatt |
| 1741 | gattgaaact ttagcatggg ctcacgaacg cacacctttg gccaatttga ttcgatggag |
| 1801 | agataaacca gtggcccttt ctattgtaca agcaagattg gttggattag ctcacttttc |
| 1861 | tgtaggttat atattcactt atgcggcttt cttgattgcc tctacgtcgg gcaaatttgg |
| 1921 | ttaattaatg tgtgtattcg cgataatctc atttctttcg acggagaagg gggtccacct |
| 1981 | tcttctattt ctacatctag gattcgactt gtatcatgga tactaatagg aattcaacca |
| 2041 | ttatggcaag gaaaagtttg attcagaggg agaagaagag gcaaaaattg gaacagaaat |
| 2101 | atcattcgat tcgtcgatcc tcaaaaaaag aaataagcaa ggttccgtcg ttgagtgaca |
| 2161 | aatgggaaat ttatggaaag ttacaatccc taccacggaa tagtgcacct acacgccttc |
| 2221 | atcgacgttg ttttttgacc ggaaggccga gagctaacta tcgagacttt ggcctatccg |
| 2281 | gacacatact tcgtgaaatg gttcatgcat gtttgttgcc aggagcaaca agatcaagtt |
| 2341 | ggtaaggatt aacgcttcat ttctatttct atggtcgatg atcatagaag cccctttacc |
| 2401 | attctgtata aatgggctat tctatttgta cagatagggt ggaggggcgc atttaatcct |
| 2461 | tgtttatcta ttagttttca gttcttatct ttggcgcggg gtagagcagt ttggtagctc |
| 2521 | gcaaggctca taaccttgag gtcacgggtt caaatcctgt ctccgcaaca tcttattctg |
| 2581 | gtctcgaggt ccgcataaag aaccacccat aatacccata atagctgttt gccaaccggt |
| 2641 | cgccgtcgtt caatgagaat ggataagagg ctcgtgggat tgacgtgagg gggcagggat |
| 2701 | gactatattt ctgggagcga actccgggcg aatatgaagc gcatggatac aagttatgcc |
| 2761 | ttggaatgaa agacaattcc gaatccgctt tgtctacgaa caaggaagct ataagtaatg |
| 2821 | caactatgaa ggatctatga ctgatgaaaa tattagacaa attgcttttt atggaaaagg |
| 2881 | aggaattgga aaatctacta cttctcaaaa tactttagct gctatggctg aaatgggaca |
| 2941 | aagaattatg attgtaggat gtgatcctaa agctgattct actagattaa tgttacattc |
| 3001 | taaagctcaa actactgtat tacatttagc tgctgaaaga ggagctgtag aagatttaga |
| 3061 | attacatgaa gtaatgttaa ctggatttag aggagtaaaa tgtgtagaat ctggaggacc |
| 3121 | tgaacctgga gtaggatgtg ctggaagagg aattattact gctattaatt ttttagaaga |
| 3181 | aaatggagct tatcaagatt tagattttgt atcttatgat gtattaggag atgtagtatg |
| 3241 | tggaggattt gctatgccta ttagagaagg aaaagctcaa gaaatttata ttgtaacttc |
| 3301 | tggagaaatg atggctatgt atgctgctaa taatattgct agaggaattt taaaatatgc |
| 3361 | tcattctgga ggagtaagat taggaggatt aatttgtaat tctagaaaag tagatagaga |
| 3421 | agatgaatta attatgaatt tagctgaaag attaaatact caaatgattc attttgtacc |
| 3481 | tagagataat attgtacaac atgctgaatt aagaagaatg actgtaaatg aatatgctcc |
| 3541 | tgattctaat caaggacaag aatatagagc tttagctaaa aaaattatta ataatgataa |
| 3601 | attaactatt cctactccta tggaaatgga tgaattagaa gctttattaa ttgaatatgg |
| 3661 | attattagat gatgatacta aacattctga aattattgga aaacctgctg aagctactaa |
| 3721 | atagcggccg caacccataa tacccataat agctgtttgc catcgctacc ttaggaccgt |
| 3781 | tatagttaac cggtggaggc agactatgac tcctcctgaa aataaaaatt tagtagatga |
| 3841 | aaataaagaa ttaattcaag aagtattaaa agcttatcct gaaaaatcta gaaaaaaaag |
| 3901 | agaaaaacat ttaaatgtac atgaagaaaa taaatctgat tgtggagtaa aatctaatat |
| 3961 | taaatctgta cctggagtaa tgactgctag aggatgtgct tatgctggat ctaaaggagt |
| 4021 | agtatgggga cctattaaag atatgattca tatttctcat ggacctgtag gatgtggata |
| 4081 | ttggtcttgg tctggaagaa gaaattatta tgtaggagta actggaatta attcttttgg |
| 4141 | aactatgcat tttacttctg attttcaaga aagagatatt gtatttggag gagataaaaa |
| 4201 | attaactaaa ttaattgaag aattagatgt attatttcct ttaaatagag gagtatctat |
| 4261 | tcaatctgaa tgtcctattg gattaattgg agatgatatt gaagctgtag ctaaaaaaac |
| 4321 | ttctaaacaa attggaaaac ctgtagtacc tttaagatgt gaaggattta gaggagtatc |
| 4381 | tcaatcttta ggacatcata ttgctaatga tgctattaga gattggattt ttcctgaata |
| 4441 | tgataaatta aaaaaagaaa atagattaga ttttgaacct tctccttatg atgtagcttt |
| 4501 | aattggagat tataatattg gaggagatgc ttgggcttct agaatgttat tagaagaaat |
| 4561 | gggattaaga gtagtagctc aatggtctgg agatggaact ttaaatgaat taattcaagg |
| 4621 | acctgctgct aaattagtat taattcattg ttatagatct atgaattata tttgtagatc |
| 4681 | tttagaagaa caatatggaa tgccttggat ggaatttaat ttttttggac ctactaaaat |
| 4741 | tgctgcttct ttaagagaaa ttgctgctaa atttgattct aaaattcaag aaaatgctga |
| 4801 | aaaagtaatt gctaaatata ctcctgtaat gaatgctgta ttagataaat atagacctag |
| 4861 | attagaagga aatactgtaa tgttatatgt aggaggatta agacctagac atgtagtacc |
| 4921 | tgcttttgaa gatttaggaa ttaaagtagt aggaactgga tatgaatttg ctcataatga |
| 4981 | tgattataaa agaactactc attatattga taatgctact attatttatg atgatgtaac |
| 5041 | tgcttatgaa tttgaagaat ttgtaaaagc taaaaaacct gatttaattg cttctggaat |
| 5101 | taaagaaaaa tatgtatttc aaaaaatggg attacctttt agacaaatgc attcttggga |
| 5161 | ttattctgga ccttatcatg gatatgatgg atttgctatt tttgctagag atatggattt |
| 5221 | agctttaaat tctcctactt ggtctttaat tggagctcct tggaaaaaag ctgctgctaa |
| 5281 | agctaaagct gctgcttaac tcgagagata caacaatgcc tcaaaatcct gaaagaactg |
| 5341 | tagatcatgt agatttattt aaacaacctg aatatactga attatttgaa aataaaagaa |
| 5401 | aaaattttga aggagctcat cctcctgaag aagtagaaag agtatctgaa tggactaaat |
| 5461 | cttgggatta tagagaaaaa aattttgcta gagaagcttt aactgtaaat cctgctaaag |
| 5521 | gatgtcaacc tgtaggagct atgtttgctg ctttaggatt tgaaggaact ttaccttttg |
| 5581 | tacaaggatc tcaaggatgt gtagcttatt ttagaactca tttatctaga cattataaag |
| 5641 | aaccttgttc tgctgtatct tcttctatga ctgaagatgc tgctgtattt ggaggattaa |
| 5701 | ataatatgat tgaaggaatg caagtatctt atcaattata taaacctaaa atgattgctg |
| 5761 | tatgtactac ttgtatggct gaagtaattg gagatgattt aggagctttt attactaatt |
| 5821 | ctaaaaatgc tggatctatt cctcaagatt ttcctgtacc ttttgctcat actccttctt |
| 5881 | ttgtaggatc tcatattact ggatatgata atatgatgaa aggaatttta tctaatttaa |
| 5941 | ctgaaggaaa aaaaaaagct acttctaatg gaaaaattaa ttttattcct ggatttgata |
| 6001 | cttatgtagg aaataataga gaattaaaaa gaatgatggg agtaatggga gtagattata |
| 6061 | ctattttatc tgattcttct gattattttg attctcctaa tatgggagaa tatgaaatgt |
| 6121 | atcctggagg aactaaatta gaagatgctg ctgattctat taatgctaaa gctactgtag |
| 6181 | ctttacaagc ttatactact cctaaaacta gagaatatat taaaactcaa tggaaacaag |
| 6241 | aaactcaagt attaagacct tttggagtaa aaggaactga tgaattttta actgctgtat |
| 6301 | ctgaattaac tggaaaagct attcctgaag aattagaaat tgaaagagga agattagtag |
| 6361 | atgctattac tgattcttat gcttggattc atggaaaaaa atttgctatt tatggagatc |
| 6421 | ctgatttaat tatttctatt acttcttttt tattagaaat gggagctgaa cctgtacata |
| 6481 | ttttatgtaa taatggagat gatactttta aaaaagaaat ggaagctatt ttagctgctt |
| 6541 | ctccttttgg aaaagaagct aaagtatgga ttcaaaaaga tttatggcat tttagatctt |
| 6601 | tattatttac tgaacctgta gattttttta ttggaaattc ttatggaaaa tatttatgga |
| 6661 | gagatacttc tattcctatg gtaagaattg gatatccttt atttgataga catcatttac |
| 6721 | atagatattc tactttagga tatcaaggag gattaaatat tttaaattgg gtagtaaata |
| 6781 | ctttattaga tgaaatggat agatctacta atattactgg aaaaactgat atttcttttg |
| 6841 | atttaattag ataggcggcc gctcgctacc ttaggaccgt tatagttatt accctgttat |
| 6901 | ccctaaccgg tggaggcttc ttgttatgac atgttttttt ggggtacagt ctatgcctcg |
| 6961 | ggcatccaag cagcaagcgc gttacgccgt gggtcgatgt ttgatgttat ggagcagcaa |
| 7021 | cgatgttacg cagcagggca gtcgccctaa aacaaagtta aacatcatgg gggaagcggt |
| 7081 | gatcgccgaa gtatcgactc aactatcaga ggtagttggc gtcatcgagc gccatctcga |
| 7141 | accgacgttg ctggccgtac atttgtacgg ctccgcagtg gatggcggcc tgaagccaca |
| 7201 | cagtgatatt gatttgctgg ttacggtgac cgtaaggctt gatgaaacaa cgcggcgagc |
| 7261 | tttgatcaac gaccttttgg aaacttcggc ttcccctgga gagagcgaga ttctccgcgc |
| 7321 | tgtagaagtc accattgttg tgcacgacga catcattccg tggcgttatc cagctaagcg |
| 7381 | cgaactgcaa tttggagaat ggcagcgcaa tgacattctt gcaggtatct tcgagccagc |
| 7441 | cacgatcgac attgatctgg ctatcttgct gacaaaagca agagaacata gcgttgcctt |
| 7501 | ggtaggtcca gcggcggagg aactctttga tccggttcct gaacaggatc tatttgaggc |
| 7561 | gctaaatgaa accttaacgc tatggaactc gccgcccgac tgggctggcg atgagcgaaa |
| 7621 | tgtagtgctt acgttgtccc gcatttggta cagcgcagta accggcaaaa tcgcgccgaa |
| 7681 | ggatgtcgct gccgactggg caatggagcg cctgccggcc cagtatcagc ccgtcatact |
| 7741 | tgaagctaga caggcttatc ttggacaaga agaagatcgc ttggcctcgc gcgcagatca |
| 7801 | gttggaagaa tttgtccact acgtgaaagg cgagatcacc aaggtagtcg gcaaataatc |
| 7861 | tcgagctcaa gcttcgaatt ctgcagtcga cggtaccgcg ggcccgggat ccacctgatc |
| 7921 | tagagtccgc aaaaatcacc agtctctctc tacaaatcta tctctctcta tttttctcca |
| 7981 | gaataatgtg tgagtagttc ccagataagg gaattagggt tcttataggg tttcgctcat |
| 8041 | gtgttgagca tataagaaac ccttagtatg tatttgtatt tgtaaaatac ttctatcaat |
| 8101 | aaaatttcta attcctaaaa ccaaaatcca gtgacgcggc cgcattaccc tgttatccct |
| 8161 | attaattaag agctcgctac cttaagagag gatatcggcg cgccgatcct agcctagtct |
| 8221 | ataggaggtt ttgaaaagaa aggagcaata atcattttct tgttctgtca agagggtgct |
| 8281 | attgctcctt tctttttttt tttttcttta ctaatttcct agtattttac tgacatagac |
| 8341 | ttttttgttt acattatcga aaaagaaaga gagggtattt gcttgcattt attcatgatg |
| 8401 | gatcccccgg gctgcaggca tgcaagctaa ttcccgatct agtaacatag atgacaccgc |
| 8461 | gcgccgcgga actagtaatt aattcccgcc tttcgctttt tgggggtgga aggcaaaaga |
| 8521 | aaacgtaggg gagggataga atcactacac tatcacggcc aactatacca actccttaat |
| 8581 | gtaaggatat atttaatgct atttatgaaa ttcaataata aaaagaaata gtaaaaaaat |
| 8641 | tactttatct tggatcttgg gcggatagcg ggaatcgaac ccgcatcttc tccttggcaa |
| 8701 | agagaaattt taccattcga ccatatccgc atttttttgt tcttgataca caatatgtac |
| 8761 | ccacatatat gatatataac cggatcttat ttgtgcagtg ccgggacaca tattctcttc |
| 8821 | ggaacgattc caataatttt gttaattata ttctttttat tcaagaagtt tgacccccct |
| 8881 | ctaatttttt tgttttcttt atttgatttg cattttcttt ggggacttag attcaaattt |
| 8941 | aatgtgtctc acaaccgaga aaaattaggg gggtcatttt ggttttgggt ctgcgacgaa |
| 9001 | taggttcaag agatgagaga attaaggata cccaccagaa agactaatcc aatccataag |
| 9061 | gaggtaccag aaaatacaac atttttgtta cttgaccagc catcaggaga agcaaataca |
| 9121 | acgggtacgc taatcaataa gattaatgaa gtagcaatta atgcaaaaac agccaattgg |
| 9181 | aaagcaagag tcatgctttt aatcctccaa gctaccaaca aatgaactat ataccatttg |
| 9241 | atccctctat cagccaaaaa atattaattg tgataaaata tgtcatcgag ggattttact |
| 9301 | ttatcatgaa tccattgatt ctatatgact tattactact ccccctttcg cactttattc |
| 9361 | gtacatggag tggggtgggg ggaaatggaa ttttcttttt tatttcacaa atggacatgc |
| 9421 | tagatcatat atctatatac ggatagatag atcgatcggc ggattcgcac ctgagatctt |
| 9481 | tctacagata gtgggggtat ccacccctat agccatgttc tattcggagg aataaaataa |
| 9541 | aaatagtctt tcggagagat ggctgagtgg ttgatagccc cggtcttgaa aaccggtata |
| 9601 | gttttgaaca aagaactatc gagggttcga atccctctct ctcctttttt gctaattgaa |
| 9661 | tagatttttt tatttagtgg ttttgcccaa tctgctatcc gaaagaaaag ggaatggctc |
| 9721 | ggctatccca cctagccaag ccagaaaaat agattagata taaattagat aaaataaatg |
| 9781 | agttgaaaaa aaaaaaagaa aaaaggaata cttaagctga ttccaagatg tatgattgaa |
| 9841 | tcaaagtaat ttgtacttca ttcaagcatt ggatctcctg tctcatatca attaagaggg |
| 9901 | gtcatggaaa gaacaggttc aaagtcgcga tcaattcctt tttcaaatcc tgctgcagcg |
| 9961 | agctccagct tttgttccct ttagtgaggg ttaattgcgc gcttggcgta atcatggtca |
| 10021 | tagctgtttc ctgtgtgaaa ttgttatccg ctcacaattc cacacaacat acgagccgga |
| 10081 | agcataaagt gtaaagcctg gggtgcctaa tgagtgagct aactcacatt aattgcgttg |
| 10141 | cgctcactgc ccgctttcca gtcgggaaac ctgtcgtgcc agctgcatta atgaatcggc |
| 10201 | caacgcgcgg ggagaggcgg tttgcgtatt gggcgctctt ccgcttcctc gctcactgac |
| 10261 | tcgctgcgct cggtcgttcg gctgcggcga gcggtatcag ctcactcaaa ggcggtaata |
| 10321 | cggttatcca cagaatcagg ggataacgca ggaaagaaca tgtgagcaaa aggccagcaa |
| 10381 | aaggccagga accgtaaaaa ggccgcgttg ctggcgtttt tccataggct ccgcccccct |
| 10441 | gacgagcatc acaaaaatcg acgctcaagt cagaggtggc gaaacccgac aggactataa |
| 10501 | agataccagg cgtttccccc tggaagctcc ctcgtgcgct ctcctgttcc gaccctgccg |
| 10561 | cttaccggat acctgtccgc ctttctccct tcgggaagcg tggcgctttc tcatagctca |
| 10621 | cgctgtaggt atctcagttc ggtgtaggtc gttcgctcca agctgggctg tgtgcacgaa |
| 10681 | ccccccgttc agcccgaccg ctgcgcctta tccggtaact atcgtcttga gtccaacccg |
| 10741 | gtaagacacg acttatcgcc actggcagca gccactggta acaggattag cagagcgagg |
| 10801 | tatgtaggcg gtgctacaga gttcttgaag tggtggccta actacggcta cactagaagg |
| 10861 | acagtatttg gtatctgcgc tctgctgaag ccagttacct tcggaaaaag agttggtagc |
| 10921 | tcttgatccg gcaaacaaac caccgctggt agcggtggtt tttttgtttg caagcagcag |
| 10981 | attacgcgca gaaaaaaagg atctcaagaa gatcctttga tcttttctac ggggtctgac |
| 11041 | gctcagtgga acgaaaactc acgttaaggg attttggtca tgagattatc aaaaaggatc |
| 11101 | ttcacctaga tccttttaaa ttaaaaatga agttttaaat caatctaaag tatatatgag |
| 11161 | taaacttggt ctgacagtta ccaatgctta atcagtgagg cacctatctc agcgatctgt |
| 11221 | ctatttcgtt catccatagt tgcctgactc cccgtcgtgt agataactac gatacgggag |
| 11281 | ggcttaccat ctggccccag tgctgcaatg ataccgcgag acccacgctc accggctcca |
| 11341 | gatttatcag caataaacca gccagccgga agggccgagc gcagaagtgg tcctgcaact |
| 11401 | ttatccgcct ccatccagtc tattaattgt tgccgggaag ctagagtaag tagttcgcca |
| 11461 | gttaatagtt tgcgcaacgt tgttgccatt gctacaggca tcgtggtgtc acgctcgtcg |
| 11521 | tttggtatgg cttcattcag ctccggttcc caacgatcaa ggcgagttac atgatccccc |
| 11581 | atgttgtgca aaaaagcggt tagctccttc ggtcctccga tcgttgtcag aagtaagttg |
| 11641 | gccgcagtgt tatcactcat ggttatggca gcactgcata attctcttac tgtcatgcca |
| 11701 | tccgtaagat gcttttctgt gactggtgag tactcaacca agtcattctg agaatagtgt |
| 11761 | atgcggcgac cgagttgctc ttgcccggcg tcaatacggg ataataccgc gccacatagc |
| 11821 | agaactttaa aagtgctcat cattggaaaa cgttcttcgg ggcgaaaact ctcaaggatc |
| 11881 | ttaccgctgt tgagatccag ttcgatgtaa cccactcgtg cacccaactg atcttcagca |
| 11941 | tcttttactt tcaccagcgt ttctgggtga gcaaaaacag gaaggcaaaa tgccgcaaaa |
| 12001 | aagggaataa gggcgacacg gaaatgttga atactcatac tcttcctttt tcaatattat |
| 12061 | tgaagcattt atcagggtta ttgtctcatg agcggataca tatttgaatg tatttagaaa |
| 12121 | aataaacaaa taggggttcc gcgcacattt ccccgaaaag tgccac |
| // | |

Sequence ID No. 2:
Tomato plastid transformation vector PGE 0011T, complete sequence
Plasmid DNA 1 . . . 12166 artificial optimizes CDS.
Tomato, Cyanobacteria other sequences; artificial sequences; vectors.

| | |
|---|---|
| DEFINITION | Tomato plastid transformation and cloning vector PGE11 |
| | complete sequence. |
| ACCESSION | PGE 0011T |
| KEYWORDS | Tomato plastid transformation; Plant Nif expression region; |
| SOURCE | Plant Genetic Engineering 0011T |
| ORGANISM | Escherichia coli XI1B |
| REFERENCE 1 | (bases 1 to 2979) |
| AUTHORS | Zaltsman Adi |
| TITLE | Plasmid for tomato stable transformation and expression of NifH |
| | and NifD-K cluster, modified plasmid vectors |
| source | 1 . . . 12166 |
| | organism = original Cloning vector pBluescript |
| | with NifH,D,K genes of Cyanobacteria and aadA and |
| | artificial sequences |
| CDS Coli BLA | complement(11181 . . . 12038) |
| rep_origin ORIE1 | complement(10404 . . . 11018) |
| misc_feature | complement(8470 . . . 9959) |
| | Tom\Chl\2 |
| misc_feature | 659 . . . 2582 |
| | /label = Tom\CHL\1 |
| CDS TRNA-G | complement(8660 . . . 8730) |
| CDS tRNA-fM | 2495 . . . 2568 |
| terminator PsbA\trminator | 8205 . . . 8399 |
| promoterrrn16P | 2644 . . . 2836 |
| CDS aadA | 6926 . . . 7855 |
| CDS NifK | 5316 . . . 6851 |
| CDS NifD | 3806 . . . 5296 |
| CDS NifH | 2837 . . . 3721 |
| BASE COUNT 3643 a 2207 c 2651 g 3665 t | |

| | |
|---|---|
| 1 | ctgaaattgt aagcgttaat attttgttaa aattcgcgtt aaatttttgt taaatcagct |
| 61 | cattttttaa ccaataggcc gaaatcggca aaatccctta taaatcaaaa gaatagaccg |
| 121 | agatagggtt gagtgttgtt ccagtttgga acaagagtcc actattaaag aacgtggact |
| 181 | ccaacgtcaa agggcgaaaa accgtctatc agggcgatgg cccactacgt gaaccatcac |
| 241 | cctaatcaag ttttttgggg tcgaggtgcc gtaaagcact aaatcggaac cctaaaggga |
| 301 | gcccccgatt tagagcttga cggggaaagc cggcgaacgt ggcgagaaag gaagggaaga |
| 361 | aagcgaaagg agcgggcgct agggcgctgg caagtgtagc ggtcacgctg cgcgtaacca |
| 421 | ccacacccgc cgcgcttaat gcgccgctac agggcgcgtc cattcgccat tcaggctgcg |
| 481 | caactgttgg gaagggcgat cggtgcgggc ctcttcgcta ttacgccagc tggcgaaagg |
| 541 | gggatgtgct gcaaggcgat taagttgggt aacgccaggg ttttcccagt cacgacgttg |
| 601 | taaaacgacg gccagtgagc gcgcgtaata cgactcacta tagggcgaat tgggtacccg |
| 661 | attggggcgt ggacataagg gtctttatga cacaatcaac aattcgcttc attttcaatt |
| 721 | aggccttgct ctagcttctt taggggttat tacttctttg gtagctcaac acatgtactc |
| 781 | tttacctgct tatgcattca tagcacaaga ctttactact caagctgcat tatataccca |
| 841 | ccaccaatat atcgcaggat tcatcatgac aggagctttt gctcatggag ctatattttt |
| 901 | cattagagat tacaatccgg agcaaaatga agataatgta ttggcaagaa tgttagatca |
| 961 | taaagaagct atcatatctc atttaagttg ggccagcctc tttctgggat tccataccct |
| 1021 | gggactttat gttcataatg atgtcatgct tgcctttggc actccggaga agcaaatctt |
| 1081 | gattgaaccg atatttgctc aatggataca atccgctcat ggtaaaactt catatgggtt |
| 1141 | cgatgtactt ttatcttcaa cgactggccc agcattcaat gcgggtcgaa gcatctggtt |
| 1201 | gccgggttgg ttaaatgctg ttaatgaaaa tagtaattca ttattcttaa caataggtcc |
| 1261 | tggagacttt ttggttcatc atgctattgc ccttggttta catacaacta cattgatctt |
| 1321 | agtaaaaggt gctttagatg cacgtggttc caagttaatg ccagataaaa aggatttcgg |
| 1381 | ttatagtttt ccgtgcgatg gcccaggacg aggcggtact tgtgatattt cggcatggga |
| 1441 | cgcgttttat ttggcagttt tttggatgtt aaatactatt ggatgggtta ctttttattg |
| 1501 | gcattggaag cacatcacat tatggcaagg taacgtttca cagtttaatg aatcttccac |
| 1561 | ttatttgatg ggctggttaa gggattattt atggttaaac tcttcacaac ttatcaatgg |
| 1621 | atataaccct tttggtatga atagtttatc ggtttgggca tggatgttct tatttggaca |
| 1681 | tcttgtttgg gctactggat ttatgttctt aatttcttgg cgtggatatt ggcaggaatt |
| 1741 | gattgaaact ttagcatggg ctcacgaacg cacacctttg gccaatttga ttcgatggag |
| 1801 | agataaacca gtggcccttt ctattgtaca agcaagattg gttggattag ctcacttttc |
| 1861 | tgtaggttat atattcactt atgcggcttt cttgattgcc tctacgtcgg gcaaatttgg |
| 1921 | ttaattaatg tgtgtattcg cgataatctc atttctttcg acggagaagg gggtccacct |
| 1981 | tcttctattt ctacatctag gattcgactt gtatcatgga tactaatagg aattcaacca |
| 2041 | ttatggcaag gaaaagtttg attcagaggg agaagaagag gcaaaaattg gaacagaaat |
| 2101 | atcattcgat tcgtcgatcc tcaaaaaaag aaataagcaa ggttccgtcg ttgagtgaca |
| 2161 | aatgggaaat ttatggaaag ttacaatccc taccacggaa tagtgcacct acacgccttc |
| 2221 | atcgacgttg ttttttgacc ggaaggccga gagctaacta tcgagacttt ggcctatccg |
| 2281 | gacacatact tcgtgaaatg gttcatgcat gtttgttgcc aggagcaaca agatcaagtt |
| 2341 | ggtaaggatt aacgcttcat ttctatttct atggtcgatg atcatagaag cccctttacc |
| 2401 | attctgtata aatgggctat tctatttgta cagatagggt ggaggggcgc atttaatcct |
| 2461 | tgtttatcta ttagttttca gttcttatct ttggcgcggg gtagagcagt ttggtagctc |
| 2521 | gcaaggctca taaccttgag gtcacgggtt caaatcctgt ctccgcaaca tcttattctg |
| 2581 | gtctcgaggt ccgcataaag aaccacccat aatacccata atagctgttt gccaaccggt |
| 2641 | cgccgtcgtt caatgagaat ggataagagg ctcgtgggat tgacgtgagg gggcagggat |
| 2701 | gactatattt ctgggagcga actccgggcg aatatgaagc gcatggatac aagttatgcc |
| 2761 | ttggaatgaa agacaattcc gaatccgctt tgtctacgaa caaggaagct ataagtaatg |
| 2821 | caactatgaa ggatctatga ctgatgaaaa tattagacaa attgcttttt atggaaaagg |
| 2881 | aggaattgga aaatctacta cttctcaaaa tactttagct gctatggctg aaatgggaca |
| 2941 | aagaattatg attgtaggat gtgatcctaa agctgattct actagattaa tgttacattc |
| 3001 | taaagctcaa actactgtat tacatttagc tgctgaaaga ggagctgtag aagatttaga |
| 3061 | attacatgaa gtaatgttaa ctggatttag aggagtaaaa tgtgtagaat ctggaggacc |
| 3121 | tgaacctgga gtaggatgtg ctggaagagg aattattact gctattaatt ttttagaaga |
| 3181 | aaatggagct tatcaagatt tagattttgt atcttatgat gtattaggag atgtagtatg |
| 3241 | tggaggattt gctatgccta ttagagaagg aaaagctcaa gaaatttata ttgtaacttc |
| 3301 | tggagaaatg atggctatgt atgctgctaa taatattgct agaggaattt taaaatatgc |
| 3361 | tcattctgga ggagtaagat taggaggatt aatttgtaat tctagaaaag tagatagaga |
| 3421 | agatgaatta attatgaatt tagctgaaag attaaatact caaatgattc attttgtacc |
| 3481 | tagagataat attgtacaac atgctgaatt aagaagaatg actgtaaatg aatatgctcc |
| 3541 | tgattctaat caaggacaag aatatagagc tttagctaaa aaaattatta ataatgataa |
| 3601 | attaactatt cctactccta tggaaatgga tgaattagaa gctttattaa ttgaatatgg |
| 3661 | attattagat gatgatacta aacattctga aattattgga aaacctgctg aagctactaa |
| 3721 | atagcggccg caacccataa tacccataat agctgtttgc catcgctacc ttaggaccgt |
| 3781 | tatagttaac cggtggaggc agactatgac tcctcctgaa aataaaaatt tagtagatga |
| 3841 | aaataaagaa ttaattcaag aagtattaaa agcttatcct gaaaaatcta gaaaaaaaag |
| 3901 | agaaaaacat ttaaatgtac atgaagaaaa taaatctgat tgtggagtaa aatctaatat |
| 3961 | taaatctgta cctggagtaa tgactgctag aggatgtgct tatgctggat ctaaaggagt |
| 4021 | agtatgggga cctattaaag atatgattca tatttctcat ggacctgtag gatgtggata |
| 4081 | ttggtcttgg tctggaagaa gaaattatta tgtaggagta actggaatta attcttttgg |
| 4141 | aactatgcat tttacttctg attttcaaga aagagatatt gtatttggag gagataaaaa |
| 4201 | attaactaaa ttaattgaag aattagatgt attatttcct ttaaatagag gagtatctat |
| 4261 | tcaatctgaa tgtcctattg gattaattgg agatgatatt gaagctgtag ctaaaaaaac |
| 4321 | ttctaaacaa attggaaaac ctgtagtacc tttaagatgt gaaggattta gaggagtatc |
| 4381 | tcaatcttta ggacatcata ttgctaatga tgctattaga gattggattt ttcctgaata |
| 4441 | tgataaatta aaaaaagaaa atagattaga ttttgaacct tctccttatg atgtagcttt |
| 4501 | aattggagat tataatattg gaggagatgc ttgggcttct agaatgttat tagaagaaat |
| 4561 | gggattaaga gtagtagctc aatggtctgg agatggaact ttaaatgaat taattcaagg |
| 4621 | acctgctgct aaattagtat taattcattg ttatagatct atgaattata tttgtagatc |
| 4681 | tttagaagaa caatatggaa tgccttggat ggaatttaat ttttttggac ctactaaaat |
| 4741 | tgctgcttct ttaagagaaa ttgctgctaa atttgattct aaaattcaag aaaatgctga |
| 4801 | aaaagtaatt gctaaatata ctcctgtaat gaatgctgta ttagataaat atagacctag |
| 4861 | attagaagga aatactgtaa tgttatatgt aggaggatta agacctagac atgtagtacc |
| 4921 | tgcttttgaa gatttaggaa ttaaagtagt aggaactgga tatgaatttg ctcataatga |
| 4981 | tgattataaa agaactactc attatattga taatgctact attatttatg atgatgtaac |
| 5041 | tgcttatgaa tttgaagaat ttgtaaaagc taaaaaacct gatttaattg cttctggaat |
| 5101 | taaagaaaaa tatgtatttc aaaaaatggg attacctttt agacaaatgc attcttggga |
| 5161 | ttattctgga ccttatcatg gatatgatgg atttgctatt tttgctagag atatggattt |
| 5221 | agctttaaat tctcctactt ggtctttaat tggagctcct tggaaaaaag ctgctgctaa |
| 5281 | agctaaagct gctgcttaac tcgagagata caacaatgcc tcaaaatcct gaaagaactg |
| 5341 | tagatcatgt agatttattt aaacaacctg aatatactga attatttgaa aataaaagaa |
| 5401 | aaaattttga aggagctcat cctcctgaag aagtagaaag agtatctgaa tggactaaat |
| 5461 | cttgggatta tagagaaaaa aattttgcta gagaagcttt aactgtaaat cctgctaaag |
| 5521 | gatgtcaacc tgtaggagct atgtttgctg ctttaggatt tgaaggaact ttaccttttg |
| 5581 | tacaaggatc tcaaggatgt gtagcttatt ttagaactca tttatctaga cattataaag |
| 5641 | aaccttgttc tgctgtatct tcttctatga ctgaagatgc tgctgtattt ggaggattaa |
| 5701 | ataatatgat tgaaggaatg caagtatctt atcaattata taaacctaaa atgattgctg |
| 5761 | tatgtactac ttgtatggct gaagtaattg gagatgattt aggagctttt attactaatt |
| 5821 | ctaaaaatgc tggatctatt cctcaagatt ttcctgtacc ttttgctcat actccttctt |
| 5881 | ttgtaggatc tcatattact ggatatgata atatgatgaa aggaatttta tctaatttaa |
| 5941 | ctgaaggaaa aaaaaaagct acttctaatg gaaaaattaa ttttattcct ggatttgata |
| 6001 | cttatgtagg aaataataga gaattaaaaa gaatgatggg agtaatggga gtagattata |
| 6061 | ctattttatc tgattcttct gattattttg attctcctaa tatgggagaa tatgaaatgt |
| 6121 | atcctggagg aactaaatta gaagatgctg ctgattctat taatgctaaa gctactgtag |
| 6181 | ctttacaagc ttatactact cctaaaacta gagaatatat taaaactcaa tggaaacaag |
| 6241 | aaactcaagt attaagacct tttggagtaa aaggaactga tgaattttta actgctgtat |
| 6301 | ctgaattaac tggaaaagct attcctgaag aattagaaat tgaaagagga agattagtag |
| 6361 | atgctattac tgattcttat gcttggattc atggaaaaaa atttgctatt tatggagatc |
| 6421 | ctgatttaat tatttctatt acttcttttt tattagaaat gggagctgaa cctgtacata |
| 6481 | ttttatgtaa taatggagat gatactttta aaaaagaaat ggaagctatt ttagctgctt |
| 6541 | ctccttttgg aaaagaagct aaagtatgga ttcaaaaaga tttatggcat tttagatctt |
| 6601 | tattatttac tgaacctgta gattttttta ttggaaattc ttatggaaaa tatttatgga |
| 6661 | gagatacttc tattcctatg gtaagaattg gatatccttt atttgataga catcatttac |
| 6721 | atagatattc tactttagga tatcaaggag gattaaatat tttaaattgg gtagtaaata |
| 6781 | ctttattaga tgaaatggat agatctacta atattactgg aaaaactgat atttcttttg |
| 6841 | atttaattag ataggcggcc gctcgctacc ttaggaccgt tatagttatt accctgttat |
| 6901 | ccctaaccgg tggaggcttc ttgttatgac atgttttttt ggggtacagt ctatgcctcg |
| 6961 | ggcatccaag cagcaagcgc gttacgccgt gggtcgatgt ttgatgttat ggagcagcaa |
| 7021 | cgatgttacg cagcagggca gtcgccctaa aacaaagtta aacatcatgg gggaagcggt |
| 7081 | gatcgccgaa gtatcgactc aactatcaga ggtagttggc gtcatcgagc gccatctcga |
| 7141 | accgacgttg ctggccgtac atttgtacgg ctccgcagtg gatggcggcc tgaagccaca |
| 7201 | cagtgatatt gatttgctgg ttacggtgac cgtaaggctt gatgaaacaa cgcggcgagc |
| 7261 | tttgatcaac gaccttttgg aaacttcggc ttcccctgga gagagcgaga ttctccgcgc |
| 7321 | tgtagaagtc accattgttg tgcacgacga catcattccg tggcgttatc cagctaagcg |
| 7381 | cgaactgcaa tttggagaat ggcagcgcaa tgacattctt gcaggtatct tcgagccagc |
| 7441 | cacgatcgac attgatctgg ctatcttgct gacaaaagca agagaacata gcgttgcctt |
| 7501 | ggtaggtcca gcggcggagg aactctttga tccggttcct gaacaggatc tatttgaggc |
| 7561 | gctaaatgaa accttaacgc tatggaactc gccgcccgac tgggctggcg atgagcgaaa |
| 7621 | tgtagtgctt acgttgtccc gcatttggta cagcgcagta accggcaaaa tcgcgccgaa |
| 7681 | ggatgtcgct gccgactggg caatggagcg cctgccggcc cagtatcagc ccgtcatact |
| 7741 | tgaagctaga caggcttatc ttggacaaga agaagatcgc ttggcctcgc gcgcagatca |
| 7801 | gttggaagaa tttgtccact acgtgaaagg cgagatcacc aaggtagtcg gcaaataatc |
| 7861 | tcgagctcaa gcttcgaatt ctgcagtcga cggtaccgcg ggcccgggat ccacctgatc |
| 7921 | tagagtccgc aaaaatcacc agtctctctc tacaaatcta tctctctcta tttttctcca |
| 7981 | gaataatgtg tgagtagttc ccagataagg gaattagggt tcttataggg tttcgctcat |
| 8041 | gtgttgagca tataagaaac ccttagtatg tatttgtatt tgtaaaatac ttctatcaat |
| 8101 | aaaatttcta attcctaaaa ccaaaatcca gtgacgcggc cgcattaccc tgttatccct |
| 8161 | attaattaag agctcgctac cttaagagag gatatcggcg cgccgatcct agcctagtct |
| 8221 | ataggaggtt ttgaaaagaa aggagcaata atcattttct tgttctgtca agagggtgct |
| 8281 | attgctcctt tctttttttt tttttcttta ctaatttcct agtattttac tgacatagac |
| 8341 | ttttttgttt acattatcga aaaagaaaga gagggtattt gcttgcattt attcatgatg |
| 8401 | gatcccccgg gctgcaggca tgcaagctaa ttcccgatct agtaacatag atgacaccgc |
| 8461 | gcgccgcgga actagtaatt aattcccgcc tttcgctttt tgggggtgga aggcaaaaga |
| 8521 | aaacgtaggg gagggataga atcactacac tatcacggcc aactatacca actccttaat |
| 8581 | gtaaggatat atttaatgct atttatgaaa ttcaataata aaaagaaata gtaaaaaaat |
| 8641 | tactttatct tggatcttgg gcggatagcg ggaatcgaac ccgcatcttc tccttggcaa |
| 8701 | agagaaattt taccattcga ccatatccgc atttttttgt tcttgataca caatatgtac |
| 8761 | ccacatatat gatatataac cggatcttat ttgtgcagtg ccgggacaca tattctcttc |
| 8821 | ggaacgattc caataatttt gttaattata ttctttttat tcaagaagtt tgacccccct |
| 8881 | ctaatttttt tgttttcttt atttgatttg cattttcttt ggggacttag attcaaattt |
| 8941 | aatgtgtctc acaaccgaga aaaattaggg gggtcatttt ggttttgggt ctgcgacgaa |
| 9001 | taggttcaag agatgagaga attaaggata cccaccagaa agactaatcc aatccataag |
| 9061 | gaggtaccag aaaatacaac atttttgtta cttgaccagc catcaggaga agcaaataca |
| 9121 | acgggtacgc taatcaataa gattaatgaa gtagcaatta atgcaaaaac agccaattgg |
| 9181 | aaagcaagag tcatgctttt aatcctccaa gctaccaaca aatgaactat ataccatttg |
| 9241 | atccctctat cagccaaaaa atattaattg tgataaaata tgtcatcgag ggattttact |
| 9301 | ttatcatgaa tccattgatt ctatatgact tattactact ccccctttcg cactttattc |
| 9361 | gtacatggag tggggtgggg ggaaatggaa ttttcttttt tatttcacaa atggacatgc |
| 9421 | tagatcatat atctatatac ggatagatag atcgatcggc ggattcgcac ctgagatctt |
| 9481 | tctacagata gtgggggtat ccacccctat agccatgttc tattcggagg aataaaataa |
| 9541 | aaatagtctt tcggagagat ggctgagtgg ttgatagccc cggtcttgaa aaccggtata |
| 9601 | gttttgaaca aagaactatc gagggttcga atccctctct ctcctttttt gctaattgaa |
| 9661 | tagatttttt tatttagtgg ttttgcccaa tctgctatcc gaaagaaaag ggaatggctc |
| 9721 | ggctatccca cctagccaag ccagaaaaat agattagata taaattagat aaaataaatg |
| 9781 | agttgaaaaa aaaaaaagaa aaaaggaata cttaagctga ttccaagatg tatgattgaa |
| 9841 | tcaaagtaat ttgtacttca ttcaagcatt ggatctcctg tctcatatca attaagaggg |
| 9901 | gtcatggaaa gaacaggttc aaagtcgcga tcaattcctt tttcaaatcc tgctgcagcg |
| 9961 | agctccagct tttgttccct ttagtgaggg ttaattgcgc gcttggcgta atcatggtca |
| 10021 | tagctgtttc ctgtgtgaaa ttgttatccg ctcacaattc cacacaacat acgagccgga |
| 10081 | agcataaagt gtaaagcctg gggtgcctaa tgagtgagct aactcacatt aattgcgttg |
| 10141 | cgctcactgc ccgctttcca gtcgggaaac ctgtcgtgcc agctgcatta atgaatcggc |
| 10201 | caacgcgcgg ggagaggcgg tttgcgtatt gggcgctctt ccgcttcctc gctcactgac |
| 10261 | tcgctgcgct cggtcgttcg gctgcggcga gcggtatcag ctcactcaaa ggcggtaata |
| 10321 | cggttatcca cagaatcagg ggataacgca ggaaagaaca tgtgagcaaa aggccagcaa |
| 10381 | aaggccagga accgtaaaaa ggccgcgttg ctggcgtttt tccataggct ccgcccccct |
| 10441 | gacgagcatc acaaaaatcg acgctcaagt cagaggtggc gaaacccgac aggactataa |
| 10501 | agataccagg cgtttccccc tggaagctcc ctcgtgcgct ctcctgttcc gaccctgccg |
| 10561 | cttaccggat acctgtccgc ctttctccct tcgggaagcg tggcgctttc tcatagctca |
| 10621 | cgctgtaggt atctcagttc ggtgtaggtc gttcgctcca agctgggctg tgtgcacgaa |
| 10681 | ccccccgttc agcccgaccg ctgcgcctta tccggtaact atcgtcttga gtccaacccg |
| 10741 | gtaagacacg acttatcgcc actggcagca gccactggta acaggattag cagagcgagg |
| 10801 | tatgtaggcg gtgctacaga gttcttgaag tggtggccta actacggcta cactagaagg |
| 10861 | acagtatttg gtatctgcgc tctgctgaag ccagttacct tcggaaaaag agttggtagc |
| 10921 | tcttgatccg gcaaacaaac caccgctggt agcggtggtt tttttgtttg caagcagcag |
| 10981 | attacgcgca gaaaaaaagg atctcaagaa gatcctttga tcttttctac ggggtctgac |
| 11041 | gctcagtgga acgaaaactc acgttaaggg attttggtca tgagattatc aaaaaggatc |
| 11101 | ttcacctaga tccttttaaa ttaaaaatga agttttaaat caatctaaag tatatatgag |
| 11161 | taaacttggt ctgacagtta ccaatgctta atcagtgagg cacctatctc agcgatctgt |
| 11221 | ctatttcgtt catccatagt tgcctgactc cccgtcgtgt agataactac gatacgggag |
| 11281 | ggcttaccat ctggccccag tgctgcaatg ataccgcgag acccacgctc accggctcca |
| 11341 | gatttatcag caataaacca gccagccgga agggccgagc gcagaagtgg tcctgcaact |
| 11401 | ttatccgcct ccatccagtc tattaattgt tgccgggaag ctagagtaag tagttcgcca |
| 11461 | gttaatagtt tgcgcaacgt tgttgccatt gctacaggca tcgtggtgtc acgctcgtcg |
| 11521 | tttggtatgg cttcattcag ctccggttcc caacgatcaa ggcgagttac atgatccccc |
| 11581 | atgttgtgca aaaaagcggt tagctccttc ggtcctccga tcgttgtcag aagtaagttg |
| 11641 | gccgcagtgt tatcactcat ggttatggca gcactgcata attctcttac tgtcatgcca |
| 11701 | tccgtaagat gcttttctgt gactggtgag tactcaacca agtcattctg agaatagtgt |
| 11761 | atgcggcgac cgagttgctc ttgcccggcg tcaatacggg ataataccgc gccacatagc |
| 11821 | agaactttaa aagtgctcat cattggaaaa cgttcttcgg ggcgaaaact ctcaaggatc |
| 11881 | ttaccgctgt tgagatccag ttcgatgtaa cccactcgtg cacccaactg atcttcagca |
| 11941 | tcttttactt tcaccagcgt ttctgggtga gcaaaaacag gaaggcaaaa tgccgcaaaa |
| 12001 | aagggaataa gggcgacacg gaaatgttga atactcatac tcttcctttt tcaatattat |
| 12061 | tgaagcattt atcagggtta ttgtctcatg agcggataca tatttgaatg tatttagaaa |
| 12121 | aataaacaaa taggggttcc gcgcacattt ccccgaaaag tgccac |
| // | |

Sequence ID No. 3:
Chlamydomonas plastid transformation vector PGE 11, complete sequence
PGE0011c Synt Chlamydomonas 11875 bp DNA circular 11875 bp DNA circular
Plasmid DNA 1 . . . 11875
Chlamydomonas, Cyanobacteria other sequences; artificial sequences; vectors.

| | |
|---|---|
| DEFINITION | Chlamydomonasplastid transformation and cloning vector PGE0011c |
| | complete sequence. |
| ACCESSION | PGE 0011c |
| VERSION | N/A |
| KEYWORDS | Chlamydomonas plastid transformation; Algae Nif expression |
| SOURCE | Plant Genetic Engineering 11c |
| ORGANISM | Escherichia coli XI1B |
| AUTHORS | Zaltsman Adi |
| TITLE | Plasmid for Chlamydomonas stable transformation and expression of NifH and |
| | NifD-K cluster, modified plasmid vectors |
| CDS Ecoli BLA | complement(10890 . . . 11747) |
| rep_origin ORIE1 | complement(10113 . . . 10727) |
| CDS aadA artificial | 6696 . . . 7625 |
| CDS NifD artificial | 3576 . . . 5066 |
| CDSNifHartificia | 2607 . . . 3491 |
| CDS NifK artificial | 5086 . . . 6621 |
| misc_featureChloroplast | 659 . . . 1998 |
| misc_featureChloroplast | 8219 . . . 9668 |
| Promoterrrn16s | 2060 . . . 2606 |
| terminator psbA | 7975 . . . 8148 |

BASE COUNT 3742 a 2024 c 2352 g 3757 t
ORIGIN

| | |
|---|---|
| 1 | ctgaaattgt aagcgttaat attttgttaa aattcgcgtt aaatttttgt taaatcagct |
| 61 | cattttttaa ccaataggcc gaaatcggca aaatccctta taaatcaaaa gaatagaccg |
| 121 | agatagggtt gagtgttgtt ccagtttgga acaagagtcc actattaaag aacgtggact |
| 181 | ccaacgtcaa agggcgaaaa accgtctatc agggcgatgg cccactacgt gaaccatcac |
| 241 | cctaatcaag ttttttgggg tcgaggtgcc gtaaagcact aaatcggaac cctaaaggga |
| 301 | gcccccgatt tagagcttga cggggaaagc cggcgaacgt ggcgagaaag gaagggaaga |
| 361 | aagcgaaagg agcgggcgct agggcgctgg caagtgtagc ggtcacgctg cgcgtaacca |
| 421 | ccacacccgc cgcgcttaat gcgccgctac agggcgcgtc cattcgccat tcaggctgcg |
| 481 | caactgttgg gaagggcgat cggtgcgggc ctcttcgcta ttacgccagc tggcgaaagg |
| 541 | gggatgtgct gcaaggcgat taagttgggt aacgccaggg ttttcccagt cacgacgttg |
| 601 | taaaacgacg gccagtgagc gcgcgtaata cgactcacta tagggcgaat tgggtaccat |
| 661 | atttaatatg gcatcaccag aggtaccaga aatgtattta cctttcctcc caaaaacact |
| 721 | tattttaaaa tctaattatt gacagaagtt gaccttgtaa atattactta aattaattta |
| 781 | gcagtatgtc aatttagtta gttatagtac cactttggga gagggtatta cattaattta |
| 841 | ttttgaaatt tacttatagt aaatagagat ttgggaacaa caactgctag cctaccgatt |
| 901 | tacaaaacgt ggttttaata atttattaat tggaaggcga ctgcaaaaga atattttgca |
| 961 | gcataggatt tatttctaat ttttctaaag ttaccaattg agttaaaaaa agctttctta |
| 1021 | attcaacatt tttaagtaaa tactgtttaa tgttatactt ttacgaatac acatatggta |
| 1081 | aaaaataaaa caatatcttt aaaataagta aaaataattt gtaaaccaat aaaaaatata |
| 1141 | tttatggtat aatataacat atgatgtaaa aaaaactatt tgtctaattt aataaccatg |
| 1201 | cattttttat gaacacataa taattaaaag cgttgctaat ggtgtaaata atgtatttat |
| 1261 | taaattaaat aattgttatt ataaggagaa atccatgaca attagtactc cagagcgcga |
| 1321 | agcgaaaaaa gtaaagattg cggttgatcg taatcctgta gaaacaagtt ttgaaaaatg |
| 1381 | ggcgtgcgac ctgaaataag gatttgacta attaaatata aaaattttta actaaaataa |
| 1441 | gctgaaaaag cttattttag ttttaagcac tgttggccga gcggatgagg caaacgactc |
| 1501 | ataatcgtta taaggtaggt tcaactccta tacggtgcaa ctaagtaaag aataagcaat |
| 1561 | acttcctttt caaaatatgg atggtcataa tatatgctat tctggtagaa tttataacaa |
| 1621 | gccttttcag aagatatata tatataactt tttatagtaa acttgttgga gatttaaatt |
| 1681 | aacatctagg agcaatcgtc cactctaatg agttaatata actctaaaag ctgaaagtaa |
| 1741 | tccatattgt tcctactccg gttttctaag ttatttttgt taaaggagtc gtctggttta |
| 1801 | ctccaacaaa agaaattggt tttatttttt ctatgtcata tacatgcgat agagtattag |
| 1861 | tttttaatac ttgcttaaaa aaaatttttg tgatataata ttattagatt aagttaatgc |
| 1921 | ggacatagct caatggtaga gtatttcctt gccaaggaaa atgttgcggg ttcgactccc |
| 1981 | gttgtccgct agaaactact cgaggtccgc ataaagaacc acccataata cccataatag |
| 2041 | ctgtttgcca accggtcgcg cctgccaact gcctatattt atatactgcg ataaacttta |
| 2101 | gtcccgaagg ggtttacata tccgaaggag gaagcaggca gtggcggtac cacgccactg |
| 2161 | gcgtcctaat ataaatattg ggcaagtaaa cttagaataa aatttatttg ctgcgttagc |
| 2221 | aggtttacat actcctaagt ttacttgccc gaaggggaag gaggacgtcc cctacgggaa |
| 2281 | tataaatatt agtggcagtg gtacaataaa taaattgtat gtaaacccct tcgggcaact |
| 2341 | aaagtttatc gcagtattaa catcctagta tataaatatc ggcagttggc aggcaacaaa |
| 2401 | tttatttatt gtcccgtaag gggaagggga aaacaattat tattttactg cggagcagct |
| 2461 | tgttattaga aatttttatt aaaaaaaaaa taaaaatttg acaaaaaaaa ataaaaaagt |
| 2521 | taaattaaaa acactgggaa tgttctaaca atcataaaaa aatcaaaagg gtttaaaatc |
| 2581 | ccgacaaaat ttaaacttta aagagtatga cagatgaaaa tattcgtcaa attgcttttt |
| 2641 | atggtaaagg tggtattggt aaatcaacaa catcacaaaa tacattagct gctatggctg |
| 2701 | aaatgggtca acgtattatg attgttggtt gtgatccaaa agctgattca acacgtttaa |
| 2761 | tgttacattc aaaagctcaa acaacagttt tacatttagc tgctgaacgt ggtgctgttg |
| 2821 | aagatttaga attacatgaa gttatgttaa caggttttcg tggtgttaaa tgtgttgaat |
| 2881 | caggtggtcc agaaccaggt gttggttgtg ctggtcgtgg tattattaca gctattaatt |
| 2941 | ttttagaaga aaatggtgct tatcaagatt tagattttgt ttcatatgat gttttaggtg |
| 3001 | atgttgtttg tggtggtttt gctatgccaa ttcgtgaagg taaagctcaa gaaatttata |
| 3061 | ttgttacatc aggtgaaatg atggctatgt atgctgctaa taatattgct cgtggtattt |
| 3121 | taaaatatgc tcattcaggt ggtgttcgtt taggtggttt aatttgtaat tcacgtaaag |
| 3181 | ttgatcgtga agatgaatta attatgaatt tagctgaacg tttaaataca caaatgattc |
| 3241 | attttgttcc acgtgataat attgttcaac atgctgaatt acgtcgtatg acagttaatg |
| 3301 | aatatgctcc agattcaaat caaggtcaag aatatcgtgc tttagctaaa aaaattatta |
| 3361 | ataatgataa attaacaatt ccaacaccaa tggaaatgga tgaattagaa gctttattaa |
| 3421 | ttgaatatgg tttattagat gatgatacaa aacattcaga aattattggt aaaccagctg |
| 3481 | aagctacaaa atagcggccg caacccataa tacccataat agctgtttgc catcgctacc |
| 3541 | ttaggaccgt tatagttaac cggtggaggc agactatgac accaccagaa aataaaaatt |
| 3601 | tagttgatga aaataaagaa ttaattcaag aagttttaaa agcttatcca gaaaaatcac |
| 3661 | gtaaaaaacg tgaaaaacat ttaaatgttc atgaagaaaa taaatcagat tgtggtgtta |
| 3721 | aatcaaatat taaatcagtt ccaggtgtta tgacagctcg tggttgtgct tatgctggtt |
| 3781 | caaaaggtgt tgtttggggt ccaattaaag atatgattca tatttcacat ggtccagttg |
| 3841 | gttgtggtta ttggtcatgg tcaggtcgtc gtaattatta tgttggtgtt acaggtatta |
| 3901 | attcatttgg tacaatgcat tttacatcag attttcaaga acgtgatatt gtttttggtg |
| 3961 | gtgataaaaa attaacaaaa ttaattgaag aattagatgt tttatttcca ttaaatcgtg |
| 4021 | gtgtttcaat tcaatcagaa tgtccaattg gtttaattgg tgatgatatt gaagctgttg |
| 4081 | ctaaaaaaac atcaaaacaa attggtaaac cagttgttcc attacgttgt gaaggttttc |
| 4141 | gtggtgtttc acaatcatta ggtcatcata ttgctaatga tgctattcgt gattggattt |
| 4201 | ttccagaata tgataaatta aaaaaagaaa atcgtttaga ttttgaacca tcaccatatg |
| 4261 | atgttgcttt aattggtgat tataatattg gtggtgatgc ttgggcttca cgtatgttat |
| 4321 | tagaagaaat gggtttacgt gttgttgctc aatggtcagg tgatggtaca ttaaatgaat |
| 4381 | taattcaagg tccagctgct aaattagttt taattcattg ttatcgttca atgaattata |
| 4441 | tttgtcgttc attagaagaa caatatggta tgccatggat ggaatttaat ttttttggtc |
| 4501 | caacaaaaat tgctgcttca ttacgtgaaa ttgctgctaa atttgattca aaaattcaag |
| 4561 | aaaatgctga aaaagttatt gctaaatata caccagttat gaatgctgtt ttagataaat |
| 4621 | atcgtccacg tttagaaggt aatacagtta tgttatatgt tggtggttta cgtccacgtc |
| 4681 | atgttgttcc agcttttgaa gatttaggta ttaaagttgt tggtacaggt tatgaatttg |
| 4741 | ctcataatga tgattataaa cgtacaacac attatattga taatgctaca attatttatg |
| 4801 | atgatgttac agcttatgaa tttgaagaat ttgttaaagc taaaaaacca gatttaattg |
| 4861 | cttcaggtat taaagaaaaa tatgtttttc aaaaaatggg tttaccattt cgtcaaatgc |
| 4921 | attcatggga ttattcaggt ccatatcatg gttatgatgg ttttgctatt tttgctcgtg |
| 4981 | atatggattt agctttaaat tcaccaacat ggtcattaat tggtgctcca tggaaaaaag |
| 5041 | ctgctgctaa agctaaagct gctgcttaac tcgagagata caacaatgcc acaaaatcca |
| 5101 | gaacgtacag ttgatcatgt tgatttattt aaacaaccag aatatacaga attatttgaa |
| 5161 | aataaacgta aaaattttga aggtgctcat ccaccagaag aagttgaacg tgtttcagaa |
| 5221 | tggacaaaat catgggatta tcgtgaaaaa aattttgctc gtgaagcttt aacagttaat |
| 5281 | ccagctaaag gttgtcaacc agttggtgct atgtttgctg ctttaggttt tgaaggtaca |
| 5341 | ttaccatttg ttcaaggttc acaaggttgt gttgcttatt ttcgtacaca tttatcacgt |
| 5401 | cattataaag aaccatgttc agctgtttca tcatcaatga cagaagatgc tgctgttttt |
| 5461 | ggtggtttaa ataatatgat tgaaggtatg caagtttcat atcaattata taaaccaaaa |
| 5521 | atgattgctg tttgtacaac atgtatggct gaagttattg gtgatgattt aggtgctttt |
| 5581 | attacaaatt caaaaaatgc tggttcaatt ccacaagatt ttccagttcc atttgctcat |
| 5641 | acaccatcat ttgttggttc acatattaca ggttatgata atatgatgaa aggtatttta |
| 5701 | tcaaatttaa cagaaggtaa aaaaaaagct acatcaaatg gtaaaattaa ttttattcca |
| 5761 | ggttttgata catatgttgg taataatcgt gaattaaaac gtatgatggg tgttatgggt |
| 5821 | gttgattata caattttatc agattcatca gattattttg attcaccaaa tatgggtgaa |
| 5881 | tatgaaatgt atccaggtgg tacaaaatta gaagatgctg ctgattcaat taatgctaaa |
| 5941 | gctacagttg ctttacaagc ttatacaaca ccaaaaacac gtgaatatat taaaacacaa |
| 6001 | tggaaacaag aaacacaagt tttacgtcca tttggtgtta aaggtacaga tgaattttta |
| 6061 | acagctgttt cagaattaac aggtaaagct attccagaag aattagaaat tgaacgtggt |
| 6121 | cgtttagttg atgctattac agattcatat gcttggattc atggtaaaaa atttgctatt |
| 6181 | tatggtgatc cagatttaat tatttcaatt acatcatttt tattagaaat gggtgctgaa |
| 6241 | ccagttcata ttttatgtaa taatggtgat gatacattta aaaaagaaat ggaagctatt |
| 6301 | ttagctgctt caccatttgg taaagaagct aaagtttgga ttcaaaaaga tttatggcat |
| 6361 | tttcgttcat tattatttac agaaccagtt gattttttta ttggtaattc atatggtaaa |
| 6421 | tatttatggc gtgatacatc aattccaatg gttcgtattg gttatccatt atttgatcgt |
| 6481 | catcatttac atcgttattc aacattaggt tatcaaggtg gtttaaatat tttaaattgg |
| 6541 | gttgttaata cattattaga tgaaatggat cgttcaacaa atattacagg taaaacagat |
| 6601 | atttcatttg atttaattcg ttaggcggcc gctcgctacc ttaggaccgt tatagttatt |
| 6661 | accctgttat ccctaaccgg tggaggcttc ttgttatgac atgttttttt ggtgttcaat |
| 6721 | caatgccacg tgcttcaaaa caacaagctc gttatgctgt tggtcgttgt ttaatgttat |
| 6781 | ggtcatcaaa tgatgttaca caacaaggtt cacgtccaaa aacaaaatta aatattatgg |
| 6841 | gtgaagctgt tattgctgaa gtttcaacac aattatcaga agttgttggt gttattgaac |
| 6901 | gtcatttaga accaacatta ttagctgttc atttatatgg ttcagctgtt gatggtggtt |
| 6961 | taaaaccaca ttcagatatt gatttattag ttacagttac agttcgttta gatgaaacaa |
| 7021 | cacgtcgtgc tttaattaat gatttattag aaacatcagc ttcaccaggt gaatcagaaa |
| 7081 | ttttacgtgc tgttgaagtt acaattgttg ttcatgatga tattattcca tggcgttatc |
| 7141 | cagctaaacg tgaattacaa tttggtgaat ggcaacgtaa tgatatttta gctggtattt |
| 7201 | ttgaaccagc tacaattgat attgatttag ctattttatt aacaaaagct cgtgaacatt |
| 7261 | cagttgcttt agttggtcca gctgctgaag aattatttga tccagttcca gaacaagatt |
| 7321 | tatttgaagc tttaaatgaa acattaacat tatggaattc accaccagat tgggctggtg |
| 7381 | atgaacgtaa tgttgtttta acattatcac gtatttggta ttcagctgtt acaggtaaaa |
| 7441 | ttgctccaaa agatgttgct gctgattggg ctatggaacg tttaccagct caatatcaac |
| 7501 | cagttatttt agaagctcgt caagcttatt taggtcaaga agaagatcgt ttagcttcac |
| 7561 | gtgctgatca attagaagaa tttgttcatt atgttaaagg tgaaattaca aaagttgttg |
| 7621 | gtaaataatc tcgagctcaa gcttcgaatt ctgcagtcga cggtaccgcg ggcccgggat |
| 7681 | ccacctgatc tagagtccgc aaaaatcacc agtctctctc tacaaatcta tctctctcta |
| 7741 | tttttctcca gaataatgtg tgagtagttc ccagataagg gaattagggt tcttataggg |
| 7801 | tttcgctcat gtgttgagca tataagaaac ccttagtatg tatttgtatt tgtaaaatac |
| 7861 | ttctatcaat aaaatttcta attcctaaaa ccaaaatcca gtgacgcggc cgcattaccc |
| 7921 | tgttatccct attaattaag agctcgctac cttaagagag gatatcggcg cgcctttttt |
| 7981 | tttaaactaa aataaatctg gttaaccata cctggtttat tttagtttat acacactttt |
| 8041 | catatatata tacttaatag ctaccatagg cagttggcag gacgtcccct tacgggacaa |
| 8101 | atgtatttat tgttgcctgc caactgccta atataaatat tagtggacgg atcccccggg |
| 8161 | ctgcaggcat gcaagctaat tcccgatcta gtaacataga tgacaccgcg cgccgcggaa |
| 8221 | gaatagaagt tacaacgtaa ttctacgttg ggttaattta ttaacccctt aaaggggttg |
| 8281 | catttagaat ccaggcatct tgggtaaatt atgtataata aaactatatt aaaagttaat |
| 8341 | tcattaaagc cgtttattta aaataaataa aaaattatgt cacgttattt aggccctcgt |
| 8401 | ttgagagtta ttcgtcgtat tggtaaatta agaggtttta cgcgtaaaaa acctttccgt |
| 8461 | cgtgtattta aaggttttgg tggttttaaa ggtaaagtta ttcctccagg tcaacatggt |
| 8521 | ttaacaaaac tattaaaaac aagaccatat gattcatctg aatcggatta tctaatccgt |
| 8581 | ttaaaagtaa aacaacgttt acgttttaac tatggtatta ctgaacgtca acttgttaac |
| 8641 | tatgtacgta aagctaaaaa aattaaagaa tctacaggtc aagttttact acaattttta |
| 8701 | gaaatgcgtt tagataatat tgtattccgt ttaaatatgg cacctacaat tccagcagca |
| 8761 | cgccaactta ttagtcatgg acatattcgt gtaaataata aaaaagtaaa tattcctagc |
| 8821 | tatatgtgta aaccaaaaga tgttatttct gtagcaatga aacaacgttc gttacaactt |
| 8881 | gtaaataaaa acttacaaga atattaccgt cgtatgcgtt tctataaaaa acgtttagaa |
| 8941 | aaaacattac cttttatttt actaaaaatt aaaccattag gtcttactag tgtaacagca |
| 9001 | gctgtagaac ttattactaa aggaaacgtg cgagtaaata ataagagtgt gaaaacgcca |
| 9061 | aattatattt gtcgtccacg agatacagtt tctttaagaa caaaacaagg tattaaaaaa |
| 9121 | gtatttttaa aaaattattt aaaaggttaa tttaatcggt aaaattccca ggtagttgca |
| 9181 | cctttccctt caggacgtcc ccttcccctt cggacaataa ataaatttgt tgcctgccaa |
| 9241 | caaatttatt tattgtatta aaatagaata aaatttattt gctgcggtag caggtttaca |
| 9301 | tacaatttat ttattgtacc actccactgg cgtccttcgg agtatgtaaa catgcttggc |
| 9361 | actggtttac atacaattta tttattgtac cactgccact gcgtcctcct tcggagtatg |
| 9421 | taaacatgct aagtttactt gcccaatatt tatattaggc agttggcagg caactcacta |
| 9481 | aaatttattt acccgaagga cgtcccgaag ggaaggggaa ggaggacgtc cccttacggg |
| 9541 | aatataaata ttagtggacg tcagtggcgg taccactgcc actggcgtcc tatatttata |
| 9601 | tactcctaag tttacttgcc caatatttat atacccgaag gaaggggaag catataaata |
| 9661 | tacttcggga gctccagctt ttgttccctt tagtgagggt taattgcgcg cttggcgtaa |
| 9721 | tcatggtcat agctgtttcc tgtgtgaaat tgttatccgc tcacaattcc acacaacata |
| 9781 | cgagccggaa gcataaagtg taaagcctgg ggtgcctaat gagtgagcta actcacatta |
| 9841 | attgcgttgc gctcactgcc cgctttccag tcgggaaacc tgtcgtgcca gctgcattaa |
| 9901 | tgaatcggcc aacgcgcggg gagaggcggt ttgcgtattg ggcgctcttc cgcttcctcg |
| 9961 | ctcactgact cgctgcgctc ggtcgttcgg ctgcggcgag cggtatcagc tcactcaaag |
| 10021 | gcggtaatac ggttatccac agaatcaggg gataacgcag gaaagaacat gtgagcaaaa |
| 10081 | ggccagcaaa aggccaggaa ccgtaaaaag gccgcgttgc tggcgttttt ccataggctc |
| 10141 | cgcccccctg acgagcatca caaaaatcga cgctcaagtc agaggtggcg aaacccgaca |
| 10201 | ggactataaa gataccaggc gtttccccct ggaagctccc tcgtgcgctc tcctgttccg |
| 10261 | accctgccgc ttaccggata cctgtccgcc tttctccctt cgggaagcgt ggcgctttct |
| 10321 | catagctcac gctgtaggta tctcagttcg gtgtaggtcg ttcgctccaa gctgggctgt |
| 10381 | gtgcacgaac cccccgttca gcccgaccgc tgcgccttat ccggtaacta tcgtcttgag |
| 10441 | tccaacccgg taagacacga cttatcgcca ctggcagcag ccactggtaa caggattagc |
| 10501 | agagcgaggt atgtaggcgg tgctacagag ttcttgaagt ggtggcctaa ctacggctac |
| 10561 | actagaagga cagtatttgg tatctgcgct ctgctgaagc cagttacctt cggaaaaaga |
| 10621 | gttggtagct cttgatccgg caaacaaacc accgctggta gcggtggttt ttttgtttgc |
| 10681 | aagcagcaga ttacgcgcag aaaaaaagga tctcaagaag atcctttgat cttttctacg |
| 10741 | gggtctgacg ctcagtggaa cgaaaactca cgttaaggga ttttggtcat gagattatca |
| 10801 | aaaaggatct tcacctagat ccttttaaat taaaaatgaa gttttaaatc aatctaaagt |
| 10861 | atatatgagt aaacttggtc tgacagttac caatgcttaa tcagtgaggc acctatctca |
| 10921 | gcgatctgtc tatttcgttc atccatagtt gcctgactcc ccgtcgtgta gataactacg |
| 10981 | atacgggagg gcttaccatc tggccccagt gctgcaatga taccgcgaga cccacgctca |
| 11041 | ccggctccag atttatcagc aataaaccag ccagccggaa gggccgagcg cagaagtggt |
| 11101 | cctgcaactt tatccgcctc catccagtct attaattgtt gccgggaagc tagagtaagt |
| 11161 | agttcgccag ttaatagttt gcgcaacgtt gttgccattg ctacaggcat cgtggtgtca |
| 11221 | cgctcgtcgt ttggtatggc ttcattcagc tccggttccc aacgatcaag gcgagttaca |
| 11281 | tgatccccca tgttgtgcaa aaaagcggtt agctccttcg gtcctccgat cgttgtcaga |
| 11341 | agtaagttgg ccgcagtgtt atcactcatg gttatggcag cactgcataa ttctcttact |
| 11401 | gtcatgccat ccgtaagatg cttttctgtg actggtgagt actcaaccaa gtcattctga |
| 11461 | gaatagtgta tgcggcgacc gagttgctct tgcccggcgt caatacggga taataccgcg |
| 11521 | ccacatagca gaactttaaa agtgctcatc attggaaaac gttcttcggg gcgaaaactc |
| 11581 | tcaaggatct taccgctgtt gagatccagt tcgatgtaac ccactcgtgc acccaactga |
| 11641 | tcttcagcat cttttacttt caccagcgtt tctgggtgag caaaaacagg aaggcaaaat |
| 11701 | gccgcaaaaa agggaataag ggcgacacgg aaatgttgaa tactcatact cttccttttt |
| 11761 | caatattatt gaagcattta tcagggttat tgtctcatga gcggatacat atttgaatgt |
| 11821 | atttagaaaa ataaacaaat aggggttccg cgcacatttc cccgaaaagt gccac |
| // | |

Sequence ID No. 4:
Promoter sequences for rrn16
4A > Chlamydomonas reinhardtii chloroplast for 16S ribosomal rrn16
Gcctgccaactgcctatatttatatactgcgataaactttagtcccgaaggggtttacatatccgaaggaggaagcaggcagtg
gcggtaccacgccactggcgtcctaatataaatattgggcaagtaaacttagaataaaatttatttgctgcgttagcaggtttacat
actcctaagtttacttgcccgaaggggaaggaggacgtcccctacgggaatataaatattagtggcagtggtacaataaataa
attgtatgtaaaccccttcgggcaactaaagtttatcgcagtattaacatcctagtatataaatatcggcagttggcaggcaacaa
atttatttattgtcccgtaaggggaaggggaaaacaattattattttactgcggagcagcttgttattagaaatttttattaaaaaaaa
aataaaaatttgacaaaaaaaaataaaaaagttaaattaaaaacactgggaatgttctaacaatcataaaaaaatcaaaag
ggtttaaaatcccgacaaaatttaaactttaaagagt
4B >Tomato Solanum lycopersicum chloroplast for 16S ribosomal rrn16
cgtcgttcaatgagaatggataagaggctcgtgggattgacgtgagggggcagggatgactatatttctgggagcgaactccg
ggcgaatatgaagcgcatggatacaagttatgccttggaatgaaagacaattccgaatccgctttgtctacgaacaaggaagc
tataagtaatgcaactatgaaggatct

TABLE 1

| Sequence ID | | | | |
|---|---|---|---|---|
| No. | Name | Sequence | Aduptor | DNA |
| 5 | TR-F | aagagctc gctgcagcaggatttgaaaaagga | SacI | Tom |
| 6 | TR-R | ttccgcgg aactagtaattaattcccgcctttcg | SacII | Tom |
| 7 | TL-F | aactcgag accagaataagatgttgcggagaca | XhoI | Tom |
| 8 | TL-F | aaaggtacc gattggggcgtggacataagg | KpnI | Tom |
| 9 | NifH atg BglII | aaaagatct atgactgacgaaaacattagacaga | BglII | Cyano |
| 10 | NifH Stop NotI | tttgcggccg ctatttggtagcttctgcgggct | NotI | Cyano |
| 11 | NifD ATG | aaaccggtgg aggcagactatgacacctcctgaaaac | AgeI | Cyano |
| 12 | NifD Stop | ttctcgag ttaggcagcagccttagcc | XhoI | Cyano |
| 13 | NifK ATG | aactcga gg agatacaacaatgcctcagaatcca | XhoI | Cyano |
| 14 | NifKstop | aaaagcggccg ctagcggatcaagtcaaaggag | NotI | Cyano |
| 15 | MCS F | aactcga gtccgcataaagaaccacccataa | XhoI | pPZP-RCS |
| 16 | MCS-R | tttccgcgg cgcgcggtgtcatctatgttacta | SacII | pPZP=RCS |
| 17 | TPsbA F | tttggatcc atcatgaataaatgcaagcaaatacc | BamHI | Tom |
| 18 | TPsbA R | tttggcgcgcc gatcctagcctagtctataggaggttt | AscI | Tom |
| 19 | Prm16S F | ccaaccggt cgccgtcgttcaatgagaat | AgeI | Tom |
| 20 | Prm16S R | tttggatcc ttcatagttgcattacttatagcttcc | BamHI | Tom |
| 21 | NifH-20 ATG | aaaagatct accaaccaattgcaggaaaagaga | BglII | Cyano |
| 22 | NifH-6 ATG | aaaagatctaggag gagaacaatgactgacgaaaacat | BglII | Cyano |
| 23 | aada ATG | aaaccggtgga ggcttcttgttatgacatgttttt | AgeI | pPZP-RCS |
| 24 | aada stop | aactcgag attatttgccgactaccttggtga | XhoI | pPZP-RCS |
| 25 | Nif H | atgactgacgaaaacattagacaga | N/A | Cyano |
| 26 | Nif-H XhoI | ctcgag-atgactgacgaaaacattagacag | XhoI | Cyano |
| 27 | Nif-H-XbaI | tctaga-atgactgacgaaaacattagacaga | XbaI | Cyano |

TABLE 2

| # | Name | Organism | Gene number | Size of CDS |
|---|---|---|---|---|
| 1 | NifH | Cyanobacteria-PCC7120/Nostoc/Anabaena | BA000019 | 888 |
| | nifH | | | |
| 2 | | Cyanobacteria-Fischerella | U49514 | 904 |
| 3 | | Cyanobacteria-Mastigocladus | DQ385928 | 804 |
| 4 | | Cyanobacteria-Nodularia | GQ456132 | 577 |
| 5 | | Cyanobacteria-Leptolyngbya | D00666 | 888 |
| 6 | | Azospirillum sp | AP010946 | 897 |
| 7 | | Zymomonas mobilis | CP002850 | 888 |
| 8 | | Rhizobium sp | HQ670655 | 894 |
| 9 | | Geobacter | CP002031 | 870 |
| 1 | NifD | Cyanobacteria-PCC7120/Nostoc/Anabaena | AF442502 | 1494 |
| | nifD | | | |
| 2 | | Cyanobacteria-Fischerella | U49514 | 1491 |
| 3 | | Cyanobacteria Xenococcus | EF576862 | 1443 |
| 4 | | Cyanobacteria-Nodularia | AF442509 | 1524 |
| 5 | | Cyanobacteria-Leptolyngbya | D00666 | 1485 |
| 6 | | Azospirillum sp | M64344 | 1449 |
| 7 | | Zymomonas mobilis | CP002865 | 1476 |
| 8 | | Rhizobium sp | X01139 | 1503 |
| 9 | | Leptothrix cholodnil | CP001013 | 1458 |
| 1 | NifK | Cyanobacteria-PCC7120/Nostoc/Anabaena | BAB73397 | 1539 |
| | nifk | | | |
| 2 | | Cyanobacteria-Fischerella | ACA61796 | 1539 |
| 3 | | Cyanobacteria-Synechococcus | SSU22146 | 1536 |
| 4 | | Cyanobacteria-Nodularia | ACA61799 | 1536 |
| 5 | | Cyanobacteria-Chlorogloeopsis | EU358079 | 1539 |
| 6 | | Azospirillum sp | AAB02344 | 149 |
| 7 | | Erwinia carotovora | BX950851 | 1569 |
| 8 | | Rhizobium sp | AAM54851 | 1542 |
| 9 | | Geobacter | YP_002138885 | 1464 |

TABLE 3
SEQ. ID. NO. 46
Plant Nuclease transformation plasmid for transit and stable
transformation NptII TP Nif HDK42 PGE # 0088

| | |
|---|---|
| FEATURES | Location/Qualifiers |
| CDS | 12469 . . . 13437 |
| | spectinomycin^{R} |
| repeat_region | 13889 . . . 14169 |
| | ORI-E1 |
| rep_origin | complement(14979 . . . 15979) |
| | PVS1-rep |
| misc_feature | 1730 . . . 1797 |
| | /label = RB |
| misc_feature | complement(12089 . . . 12132) |
| | LB |
| CDS | complement(2544 . . . 3335) |
| | Reporter NptII |
| Artificial CDS | 4661 . . . 5848 |
| | TP-NifH42 |
| Artificial CDS | 9951 . . . 11690 |
| | TP-NifK42 |
| Artificial CDS | 7041 . . . 8795 |
| | TP-NifD42 |
| promoter | 3771 . . . 4660 |
| | 2X35SP |
| promoter | 6151 . . . 7037 |
| | 2X35SP |
| promoter | 9057 . . . 9947 |
| | 2X35SP |

| | |
|---|---|
| 1 | tgcgcgtgcc tttgatcgcc cgcgacacga caaaggccgc ttgtagcctt ccatccgtga |
| 61 | cctcaatgcg ctgcttaacc agctccacca ggtcggcggt ggcccatatg tcgtaagggc |
| 121 | ttggctgcac cggaatcagc acgaagtcgg ctgccttgat cgcggacaca gccaagtccg |
| 181 | ccgcctgggg cgctccgtcg atcactacga agtcgcgccg gccgatggcc ttcacgtcgc |
| 241 | ggtcaatcgt cgggcggtcg atgccgacaa cggttagcgg ttgatcttcc cgcacggccg |
| 301 | cccaatcgcg ggcactgccc tggggatcgg aatcgactaa cagaacatcg gccccggcga |
| 361 | gttgcagggc gcgggctaga tgggttgcga tggtcgtctt gcctgacccg cctttctggt |
| 421 | taagtacagc gataaccttc atgcgttccc cttgcgtatt tgtttattta ctcatcgcat |
| 481 | catatacgca gcgaccgcat gacgcaagct gttttactca aatacacatc acctttttag |
| 541 | acggcggcgc tcggtttctt cagcggccaa gctggccggc caggccgcca gcttggcatc |
| 601 | agacaaaccg gccaggattt catgcagccg cacggttgag acgtgcgcgg gcggctcgaa |
| 661 | cacgtacccg gccgcgatca tctccgcctc gatctcttcg gtaatgaaaa acggttcgtc |
| 721 | ctggccgtcc tggtgcggtt tcatgcttgt tcctcttggc gttcattctc ggcggccgcc |
| 781 | agggcgtcgg cctcggtcaa tgcgtcctca cggaaggcac cgcgccgcct ggcctcggtg |
| 841 | ggcgtcactt cctcgctgcg ctcaagtgcg cggtacaggg tcgagcgatg cacgccaagc |
| 901 | agtgcagccg cctctttcac ggtgcggcct tcctggtcga tcagctcgcg ggcgtgcgcg |
| 961 | atctgtgccg gggtgagggt agggcggggg ccaaacttca cgcctcgggc cttggcggcc |
| 1021 | tcgcgcccgc tccgggtgcg gtcgatgatt agggaacgct cgaactcggc aatgccggcg |
| 1081 | aacacggtca acaccatgcg gccggccggc gtggtggtgt cggcccacgg ctctgccagg |
| 1141 | ctacgcaggc ccgcgccggc ctcctggatg cgctcggcaa tgtccagtag gtcgcgggtg |
| 1201 | ctgcgggcca ggcggtctag cctggtcact gtcacaacgt cgccagggcg taggtggtca |
| 1261 | agcatcctgg ccagctccgg gcggtcgcgc ctggtgccgg tgatcttctc ggaaaacagc |
| 1321 | ttggtgcagc cggccgcgtg cagttcggcc cgttggttgg tcaagtcctg gtcgtcggtg |
| 1381 | ctgacgcggg catagcccag caggccagcg gcggcgctct tgttcatggc gtaatgtctc |
| 1441 | cggttctagt cgcaagtatt ctactttatg cgactaaaac acgcgacaag aaaacgccag |
| 1501 | gaaaagggca gggcggcagc ctgtcgcgta acttaggact tgtgcgacat gtcgttttca |
| 1561 | gaagacggct gcactgaacg tcagaagccg actgcactat agcagcggag gggttggatc |
| 1621 | aaagtacttt gatcccgagg ggaaccctgt ggttggcatg cacatacaaa tggacgaacg |
| 1681 | gataaacctt ttcacgccct tttaaatatc cgttattcta ataaacgctc ttttctctta |
| 1741 | ggtttacccg ccaatatatc ctgtcaaaca ctgatagttt aaactgaagg cgggaaacga |
| 1801 | caatctgatc caagctcaag ctaagcttca gagccaccac cctcagagcc gccaccagaa |
| 1861 | ccaccaccag agccgccgcc agcattgaca ggaggcccga tctagtaaca tagatgacac |
| 1921 | cgcgcgcgat aatttatcct agtttgcgcg ctatattttg ttttctatcg cgtattaaat |
| 1981 | gtataattgc gggactctaa tcataaaaac ccatctcata aataacgtca tgcattacat |
| 2041 | gttaattatt acatgcttaa cgtaattcaa cagaaattat atgataatca tcgcaagacc |
| 2101 | ggcaacagga ttcaatctta agaaacttta ttgccaaatg tttgaacgat cggggatcat |
| 2161 | ccgggtctgt ggcgggaact ccacgaaaat atccgaacgc agcaagatat cgcggtccat |
| 2221 | ctcggtcttg cctgggcagt cgccgccgac gccgttgatg tggacgccgg gcccgatcat |
| 2281 | attgtcgctc aggatcgtgg cgttgtgctt gtcggccgtt gctgtcgtaa tgatatcggc |
| 2341 | accttcgacc gcctgttccg cagagatccc gtgggcgaag aactccagca tgagatcccc |
| 2401 | gcgctggagg atcatccagc cggcgtcccg gaaaacgatt ccgaagccca acctttcata |
| 2461 | gaaggcggcg gtggaatcga aatctcgtga tggcaggttg ggcgtcgctt ggtcggtcat |
| 2521 | ttcgaacccc agagtcccgc tcagaagaac tcgtcaagaa ggcgatagaa ggcgatgcgc |
| 2581 | tgcgaatcgg gagcggcgat accgtaaagc acgaggaagc ggtcagccca ttcgccgcca |
| 2641 | agctcttcag caatatcacg ggtagccaac gctatgtcct gatagcggtc cgccacaccc |
| 2701 | agccggccac agtcgatgaa tccagaaaag cggccatttt ccaccatgat attcggcaag |
| 2761 | caggcatcgc catgggtcac gacgagatca tcgccgtcgg gcatgcgcgc cttgagcctg |
| 2821 | gcgaacagtt cggctggcgc gagcccctga tgctcttcgt ccagatcatc ctgatcgaca |
| 2881 | agaccggctt ccatccgagt acgtgctcgc tcgatgcgat gtttcgcttg gtggtcgaat |
| 2941 | gggcaggtag ccggatcaag cgtatgcagc cgccgcattg catcagccat gatggatact |
| 3001 | ttctcggcag gagcaaggtg agatgacagg agatcctgcc ccggcacttc gcccaatagc |
| 3061 | agccagtccc ttcccgcttc agtgacaacg tcgagcacag ctgcgcaagg aacgcccgtc |
| 3121 | gtggccagcc acgatagccg cgctgcctcg tcctgcagtt cattcagggc accggacagg |
| 3181 | tcggtcttga caaaaagaac cgggcgcccc tgcgctgaca gccggaacac ggcggcatca |
| 3241 | gagcagccga ttgtctgttg tgcccagtca tagccgaata gcctctccac ccaagcggcc |
| 3301 | ggagaacctg cgtgcaatcc atcttgttca atcatgcgaa acgatccaga tccggtgcag |
| 3361 | attatttgga ttgagagtga atatgagact ctaattggat accgagggga atttatggaa |
| 3421 | cgtcagtgga gcatttttga caagaaatat ttgctagctg atagtgacct taggcgactt |
| 3481 | ttgaacgcgc aataatggtt tctgacgtat gtgcttagct cattaaactc cagaaacccg |
| 3541 | cggctgagtg gctccttcaa cgttgcggtt ctgtcagttc caaacgtaaa acggcttgtc |
| 3601 | ccgcgtcatc ggcgggggtc ataacgtgac tcccttaatt ctccgctcat gatcagattg |
| 3661 | tcgtttcccg ccttcaggta ccgcgatcgc tcgcgacctg caggcataaa gccgtcagtg |
| 3721 | tccgcataaa gaaccaaccc ataataccca taatagctgt ttgccaaccg gtcaacatgt |
| 3781 | ggagcacgac acacttgtct actccaaaaa tatcaaagat acagtctcag aagaccaaag |
| 3841 | ggcaattgag acttttcaac aaagggtaat atccggaaac ctcctcggat tccattgccc |
| 3901 | agctatctgt cactttattg tgaagatagt ggaaaaggaa ggtggctcct acaaatgcca |
| 3961 | tcattgcgat aaaggaaagg ccatcgttga agatgcctct gccgacagtg gtcccaaaga |
| 4021 | tggaccccca cccacgagga gcatcgtgga aaaagaagac gttccaacca cgtcttcaaa |
| 4081 | gcaagtggat tgatgtgata acatggtgga gcacgacaca cttgtctact ccaaaaatat |
| 4141 | caaagataca gtctcagaag accaaagggc aattgagact tttcaacaaa gggtaatatc |
| 4201 | cggaaacctc ctcggattcc attgcccagc tatctgtcac tttattgtga agatagtgga |
| 4261 | aaaggaaggt ggctcctaca aatgccatca ttgcgataaa ggaaaggcca tcgttgaaga |
| 4321 | tgcctctgcc gacagtggtc ccaaagatgg acccccaccc acgaggagca tcgtggaaaa |
| 4381 | agaagacgtt ccaaccacgt cttcaaagca agtggattga tgtgatatct ccactgacgt |
| 4441 | aagggatgac gcacaatccc actatccttc gcaagaccct tcctctatat aaggaagttc |
| 4501 | atttcatttg gagaggacgt cgagagttct caacacaaca tatacaaaac aaacgaatct |
| 4561 | caagcaatca agcattctac ttctattgca gcaatttaaa tcatttcttt taaagcaaaa |
| 4621 | gcaattttct gaaaattttc accatttacg aacgatagcc atggctagcg ctaccggact |
| 4681 | cagatctatg gcttcctctg tcatttcttc agcagctgtt gccacacgca gcaatgttac |
| 4741 | acaagctagc atggttgcac ctttcactgg tctcaaatct tcagccactt tccctgttac |
| 4801 | aaagaagcaa aaccttgaca tcacttccat tgctagcaat ggtggaagag ttagctgcag |
| 4861 | agctcaaatg ggccgcaaca gccagggctt cctgacgacg atccgcatca tcgtcacgag |
| 4921 | cgcgacggac cagccgagca acttcatcca cagcctgagc aacaagcgcg agagcacgat |
| 4981 | gcgccagatc gcgttctacg gcaagggcgg catcggcaag agcacgacga gccagaacac |
| 5041 | gatcgcggcg ctggcggaga cgaaccgcat catgatcgtc ggctgcgacc cgaaggcgga |
| 5101 | cagcacgcgc ctgatgctgc acacgaaggc gcagacgacg atcctgcacc tggcggcgga |
| 5161 | gcgcggcacg gtcgaggaca tcgagctgga ggaggtcctg ctggagggct accagggcgt |
| 5221 | caagtgcgtc gagagcggcg gcccggagcc gggcgtcggc tgcgcgggcc gcggcatcat |
| 5281 | cacggcgatc aacttcctgg aggaggaggg cgcgtacgag gacctggact tcgtcagcta |
| 5341 | cgacgtcctg ggcgacgtcg tctgcggcgg cttcgcgatg ccgatccgcg agggcaaggc |
| 5401 | gcaggagatc tacatcgtca cgagcggcga gatgatggcg atgtacgcgg cgaacaacat |
| 5461 | cgcgcgcggc atcctgaagt acgcgcacac gggcggcgtc cgcctgggcg gcctgatctg |
| 5521 | caacagccgc aacgtcaact gcgaggcgga gctgatcgag gagctggcgc gccgcctggg |
| 5581 | cacgcagatg atccacttcg tcccgcgcag caagcaggtc caggaggcgg agctgcgccg |
| 5641 | catgacggtc atcgagtaca gcccggacca cccgcaggcg caggagtacc gcgagctgag |
| 5701 | ccgcaagatc gagaacaaca cgaacctggt catcccgacg ccgatcacga tggaggagct |
| 5761 | ggaggagctg ctggtcgact tcggcatcct gggcggcgag gacgagtacg agaaggcgct |
| 5821 | gcaggcggac aaggcggcga cgaaggcgta gtaggatcca cctgatctag agtccgcaaa |
| 5881 | aatcaccagt ctctctctac aaatctatct ctctctattt ttctccagaa taatgtgtga |
| 5941 | gtagttccca gataagggaa ttagggttct tatagggttt cgctcatgtg ttgagcatat |
| 6001 | aagaaaccct tagtatgtat ttgtatttgt aaaatacttc tatcaataaa atttctaatt |
| 6061 | cctaaaacca aaatccagtg acgcggccgc acccataata cccataatag ctgtttgcca |
| 6121 | tcgctacctt aggaccgtta tagttaaccg gtcaacatgt ggagcacgac acacttgtct |
| 6181 | actccaaaaa tatcaaagat acagtctcag aagaccaaag ggcaattgag acttttcaac |
| 6241 | aaagggtaat atccggaaac ctcctcggat tccattgccc agctatctgt cactttattg |
| 6301 | tgaagatagt ggaaaaggaa ggtggctcct acaaatgcca tcattgcgat aaaggaaagg |
| 6361 | ccatcgttga agatgcctct gccgacagtg gtcccaaaga tggaccccca cccacgagga |
| 6421 | gcatcgtgga aaaagaagac gttccaacca cgtcttcaaa gcaagtggat tgatgtgata |
| 6481 | acatggtgga gcacgacaca cttgtctact ccaaaaatat caaagataca gtctcagaag |
| 6541 | accaaagggc aattgagact tttcaacaaa gggtaatatc cggaaacctc ctcggattcc |
| 6601 | attgcccagc tatctgtcac tttattgtga agatagtgga aaaggaaggt ggctcctaca |
| 6661 | aatgccatca ttgcgataaa ggaaaggcca tcgttgaaga tgcctctgcc gacagtggtc |
| 6721 | ccaaagatgg acccccaccc acgaggagca tcgtggaaaa agaagacgtt ccaaccacgt |
| 6781 | cttcaaagca agtggattga tgtgatatct ccactgacgt aagggatgac gcacaatccc |
| 6841 | actatccttc gcaagaccct tcctctatat aaggaagttc atttcatttg gagaggacgt |
| 6901 | cgagagttct caacacaaca tatacaaaac aaacgaatct caagcaatca agcattctac |
| 6961 | ttctattgca gcaatttaaa tcatttcttt taaagcaaaa gcaattttct gaaaattttc |
| 7021 | accatttacg aacgatagcc atggctagcg ctaccggact cagatctatg gcttcctctg |
| 7081 | tcatttcttc agcagctgtt gccacacgca gcaatgttac acaagctagc atggttgcac |
| 7141 | ctttcactgg tctcaaatct tcagccactt tccctgttac aaagaagcaa aaccttgaca |
| 7201 | tcacttccat tgctagcaat ggtggaagag ttagctgcag agctcaaatg gttgcacctt |
| 7261 | tcactggtct caaatcttca gccactttcc ctgttacaaa gaagcaaaac cttgacatca |
| 7321 | cttccattgc tagcaatggt ggaagagtta gctggatggc tactgtagaa gataataaaa |
| 7381 | aattaattgc tgatgtatta tctacttatc ctgaaaaagc tgctaaaaaa agagctaaac |
| 7441 | atttaggagt atatgaagaa ggagaagctg attgtggagt aaaatctaat aaacaatctt |
| 7501 | tacctggagt aatgactgct agaggatgtg cttatgctgg atctaaagga gtagtatggg |
| 7561 | gacctattaa agatatggta catatttctc atggacctgt aggatgtgga tattattctt |
| 7621 | ggtctggaag aagaaattat tatattggaa ctactggagt agattctttt ggaactatgc |
| 7681 | aatttacttc tgattttcaa gaaagagata ttgtatttgg aggagataaa aaattagcta |
| 7741 | aaattattga tgaaattgaa gaattatttc ctttaaatgg aggagtatct gtacaatctg |
| 7801 | aatgtcctgt aggattaatt ggagatgata ttgaatctgt agctagaact aaatctaaag |
| 7861 | aaactggaaa atctgtagta cctgtaagat gtgaaggatt tagaggagta tctcaatctt |
| 7921 | taggacatca tattgctaat gatatgatta gagattgggt atttcctact gctgataaag |
| 7981 | aaaatgctga aaaaggattt gaaggaactc cttatgatgt agctattatt ggagattata |
| 8041 | atattggagg agatgcttgg tcttctagaa ttttattaga agaaattgga ttaagagtag |
| 8101 | tagctcaatg gtctggagat ggaactttaa ctgaaatgaa agctactcct aatgtaaaat |
| 8161 | taaatttaat tcattgttat agatctatga attatatttc tagacatatg gaagaaaaat |
| 8221 | atggaattcc ttggttagaa tataattttt ttggaccttc taaaattgct gcttctttaa |
| 8281 | gagaaattgc ttctagattt gatgaaaaaa ttcaagctaa agctgaagaa gtaattgaaa |
| 8341 | aatatagaaa acaatctgaa gaaattattg ctaaatatag acctagatta gaaggaaaaa |
| 8401 | ctgtaatgat gatggtagga ggattaagac ctagacatgt agtacctgct tttaaagatt |
| 8461 | taggaatgga aattattgga actggatatg aatttgctca tggagatgat tataaaagaa |
| 8521 | ctactggata tgtagaagat gctactttaa tttatgatga tgtaactgga tatgaatttg |
| 8581 | aagaatttgt aaaagaatta aaacctgatt tagtagctgc tggaattaaa gaaaaatatg |
| 8641 | tatttcaaaa aatggcttta ccttttagac aaatgcattc ttgggattat tctggacctt |
| 8701 | atcatggata tgatggattt gctatttttg ctagagatat ggatttagct ttaaattctc |
| 8761 | ctacttgggg attaattgga actccttgga ataaataagt aggatccacc tgatctagag |
| 8821 | tccgcaaaaa tcaccagtct ctctctacaa atctatctct ctctattttt ctccagaata |
| 8881 | atgtgtgagt agttcccaga taagggaatt agggttctta tagggtttcg ctcatgtgtt |
| 8941 | gagcatataa gaaaccctta gtatgtattt gtatttgtaa aatacttcta tcaataaaat |
| 9001 | ttctaattcc taaaaccaaa atccagtgac gcggccgcat taccctgtta tccctaaccg |
| 9061 | gtcaacatgt ggagcacgac acacttgtct actccaaaaa tatcaaagat acagtctcag |
| 9121 | aagaccaaag ggcaattgag acttttcaac aaagggtaat atccggaaac ctcctcggat |
| 9181 | tccattgccc agctatctgt cactttattg tgaagatagt ggaaaaggaa ggtggctcct |
| 9241 | acaaatgcca tcattgcgat aaaggaaagg ccatcgttga agatgcctct gccgacagtg |
| 9301 | gtcccaaaga tggaccccca cccacgagga gcatcgtgga aaaagaagac gttccaacca |
| 9361 | cgtcttcaaa gcaagtggat tgatgtgata acatggtgga gcacgacaca cttgtctact |
| 9421 | ccaaaaatat caaagataca gtctcagaag accaaagggc aattgagact tttcaacaaa |
| 9481 | gggtaatatc cggaaacctc ctcggattcc attgcccagc tatctgtcac tttattgtga |
| 9541 | agatagtgga aaaggaaggt ggctcctaca aatgccatca ttgcgataaa ggaaaggcca |
| 9601 | tcgttgaaga tgcctctgcc gacagtggtc ccaaagatgg acccccaccc acgaggagca |
| 9661 | tcgtggaaaa agaagacgtt ccaaccacgt cttcaaagca agtggattga tgtgatatct |
| 9721 | ccactgacgt aagggatgac gcacaatccc actatccttc gcaagaccct tcctctatat |
| 9781 | aaggaagttc atttcatttg gagaggacgt cgagagttct caacacaaca tatacaaaac |
| 9841 | aaacgaatct caagcaatca agcattctac ttctattgca gcaatttaaa tcatttcttt |
| 9901 | taaagcaaaa gcaattttct gaaaattttc accatttacg aacgatagcc atggctagcg |
| 9961 | ctaccggact cagatctatg gcttcctctg tcatttcttc agcagctgtt gccacacgca |
| 10021 | gcaatgttac acaagctagc atggttgcac ctttcactgg tctcaaatct tcagccactt |
| 10081 | tccctgttac aaagaagcaa aaccttgaca tcacttccat tgctagcaat ggtggaagag |
| 10141 | ttagctgcag agctcaaatg gctcaaaatg taaataatat taaagatcat gtagatttat |
| 10201 | ttcatcaacc tgaatatcaa gaattatttg aaaataaaaa acaatttgaa ggaatgccta |
| 10261 | ctgctgaaaa agtacaagaa gtagctgaat ggactaaatc ttgggaatat agagaaaaaa |
| 10321 | attttgctag agaagcttta actgtaaatc ctgctaaagc ttgtcaacct ttaggagctt |
| 10381 | tattagctgc tattggattt gaaggaactt taccttttgt acatggatct caaggatgtg |
| 10441 | tagcttattt tagaactcat ttaactagac attttaaaga acctgtatct gctgtatctt |
| 10501 | cttctatgac tgaaaatgct gctgtatttg gaggattaaa aaatatggta gatggattac |
| 10561 | aaaattctta tgctttatat aaacctaaaa tgattgctgt atgtactact tgtatggctg |
| 10621 | aagtaattgg agatgattta ggagcttttt taggaaatgc tagacaagat ggagtaattc |
| 10681 | ctgatgattt acctgtacct tttgctcata ctccttcttt tgtaggatct catattactg |
| 10741 | gatatgattc tatgatgaaa tctattttat ctactttaac tgaaggaaaa aaaaaagaaa |
| 10801 | ctactaatgg aaaaattaat tttattgctg gatttgaaac ttatattgga aatgtaagag |
| 10861 | ctattaaaaa tattatttct gcttttgatt tagaaggaac tttattatct gatactgaaa |
| 10921 | tgtatttaga ttctcctaat ttaggagaat ttaaaatgta tcatgaagga acttctttag |
| 10981 | aagatgctgc tgattctatt aatgctgaag ctactgtaac tttacaaact tatactactc |
| 11041 | ctaaaactag agaatatatt gaaaaaaaat ggggacaaaa aacttatact tatagacctt |
| 11101 | ggggagtaaa aggaactgat gaatttttaa tgggattatc tgaattaact ggaaaaccta |
| 11161 | ttcctaaaga atttgaaatt gctagaggaa gagctgtaga tgctatgact gatactcaag |
| 11221 | cttgggtaca tggaaaaaga gctgctgtat atggagatcc tgatttagta atgggattat |
| 11281 | tacaatttat gttagaaatg ggaattgaac ctgtacatgt attagtaaat aattctacta |
| 11341 | aagaatttga agaagaagct aaagctttat tagctgcttc tccttatgga caacaagcta |
| 11401 | ctgtatgggg aggaaaagat ttatggcata tgagatcttt attatttact gaacctgtag |
| 11461 | attttttagt aggaaattct tatgctaaat atttacaaag agatactaaa actcctttaa |
| 11521 | ttagaattgg atatcctatt tttgatagac atcatttaca tagatattct actattggat |
| 11581 | atgaaggagc tattaattta ttaaattgga ttgctaatgg attaatggat gaattagata |
| 11641 | gaaaaactga tactccttct gtaactgata tttcttttga tttagtaaga taggatccac |
| 11701 | ctgatctaga gtccgcaaaa atcaccagtc tctctctaca aatctatctc tctctatttt |
| 11761 | tctccagaat aatgtgtgag tagttcccag ataagggaat tagggttctt atagggtttc |
| 11821 | gctcatgtgt tgagcatata agaaaccctt agtatgtatt tgtatttgta aaatacttct |
| 11881 | atcaataaaa tttctaattc ctaaaaccaa aatccagtga cgcggccgca ttaccctgtt |
| 11941 | atccctatta attaagagct cgctacctta agagaggata tcggcgcgcc tctagaattt |
| 12001 | aaatggatcc tacgtactcg aggaattcaa ttcggcgtta attcagtaca ttaaaaacgt |
| 12061 | ccgcaatgtg ttattaagtt gtctaagcgt caatttgttt acaccacaat atatcctgcc |
| 12121 | accagccagc caacagctcc ccgaccggca gctcggcaca aaatcaccac tcgatacagg |
| 12181 | cagcccatca gtccgggacg gcgtcagcgg gagagccgtt gtaaggcggc agactttgct |
| 12241 | catgttaccg atgctattcg gaagaacggc aactaagctg ccgggtttga aacacggatg |
| 12301 | atctcgcgga gggtagcatg ttgattgtaa cgatgacaga gcgttgctgc ctgtgatcaa |
| 12361 | ttcgggcacg aacccagtgg acataagcct cgttcggttc gtaagctgta atgcaagtag |
| 12421 | cgtaactgcc gtcacgcaac tggtccagaa ccttgaccga acgcagcggt ggtaacggcg |
| 12481 | cagtggcggt tttcatggct tcttgttatg acatgttttt ttggggtaca gtctatgcct |
| 12541 | cgggcatcca agcagcaagc gcgttacgcc gtgggtcgat gtttgatgtt atggagcagc |
| 12601 | aacgatgtta cgcagcaggg cagtcgccct aaaacaaagt taaacatcat gggggaagcg |
| 12661 | gtgatcgccg aagtatcgac tcaactatca gaggtagttg gcgtcatcga gcgccatctc |
| 12721 | gaaccgacgt tgctggccgt acatttgtac ggctccgcag tggatggcgg cctgaagcca |
| 12781 | cacagtgata ttgatttgct ggttacggtg accgtaaggc ttgatgaaac aacgcggcga |
| 12841 | gctttgatca acgacctttt ggaaacttcg gcttcccctg gagagagcga gattctccgc |
| 12901 | gctgtagaag tcaccattgt tgtgcacgac gacatcattc cgtggcgtta tccagctaag |
| 12961 | cgcgaactgc aatttggaga atggcagcgc aatgacattc ttgcaggtat cttcgagcca |
| 13021 | gccacgatcg acattgatct ggctatcttg ctgacaaaag caagagaaca tagcgttgcc |
| 13081 | ttggtaggtc cagcggcgga ggaactcttt gatccggttc ctgaacagga tctatttgag |
| 13141 | gcgctaaatg aaaccttaac gctatggaac tcgccgcccg actgggctgg cgatgagcga |
| 13201 | aatgtagtgc ttacgttgtc ccgcatttgg tacagcgcag taaccggcaa aatcgcgccg |
| 13261 | aaggatgtcg ctgccgactg ggcaatggag cgcctgccgg cccagtatca gcccgtcata |
| 13321 | cttgaagcta gacaggctta tcttggacaa gaagaagatc gcttggcctc gcgcgcagat |
| 13381 | cagttggaag aatttgtcca ctacgtgaaa ggcgagatca ccaaggtagt cggcaaataa |
| 13441 | tgtctagcta gaaattcgtt caagccgacg ccgcttcgcc ggcgttaact caagcgatta |
| 13501 | gatgcactaa gcacataatt gctcacagcc aaactatcag gtcaagtctg cttttattat |
| 13561 | ttttaagcgt gcataataag ccctacacaa attgggagat atatcatgca tgaccaaaat |
| 13621 | cccttaacgt gagttttcgt tccactgagc gtcagacccc gtagaaaaga tcaaaggatc |
| 13681 | ttcttgagat cctttttttc tgcgcgtaat ctgctgcttg caaacaaaaa aaccaccgct |
| 13741 | accagcggtg gtttgtttgc cggatcaaga gctaccaact ctttttccga aggtaactgg |
| 13801 | cttcagcaga gcgcagatac caaatactgt ccttctagtg tagccgtagt taggccacca |
| 13861 | cttcaagaac tctgtagcac cgcctacata cctcgctctg ctaatcctgt taccagtggc |
| 13921 | tgctgccagt ggcgataagt cgtgtcttac cgggttggac tcaagacgat agttaccgga |
| 13981 | taaggcgcag cggtcgggct gaacgggggg ttcgtgcaca cagcccagct tggagcgaac |
| 14041 | gacctacacc gaactgagat acctacagcg tgagctatga gaaagcgcca cgcttcccga |
| 14101 | agggagaaag gcggacaggt atccggtaag cggcagggtc ggaacaggag agcgcacgag |
| 14161 | ggagcttcca gggggaaacg cctggtatct ttatagtcct gtcgggtttc gccacctctg |
| 14221 | acttgagcgt cgatttttgt gatgctcgtc aggggggcgg agcctatgga aaaacgccag |
| 14281 | caacgcggcc tttttacggt tcctggcctt ttgctggcct tttgctcaca tgttctttcc |
| 14341 | tgcgttatcc cctgattctg tggataaccg tattaccgcc tttgagtgag ctgataccgc |
| 14401 | tcgccgcagc cgaacgaccg agcgcagcga gtcagtgagc gaggaagcgg aagagcgcct |
| 14461 | gatgcggtat tttctcctta cgcatctgtg cggtatttca caccgcatat ggtgcactct |
| 14521 | cagtacaatc tgctctgatg ccgcatagtt aagccagtat acactccgct atcgctacgt |
| 14581 | gactgggtca tggctgcgcc ccgacacccg ccaacacccg ctgacgcgcc ctgacgggct |
| 14641 | tgtctgctcc cggcatccgc ttacagacaa gctgtgaccg tctccgggag ctgcatgtgt |
| 14701 | cagaggtttt caccgtcatc accgaaacgc gcgaggcagg gtgccttgat gtgggcgccg |
| 14761 | gcggtcgagt ggcgacggcg cggcttgtcc gcgccctggt agattgcctg gccgtaggcc |
| 14821 | agccattttt gagcggccag cggccgcgat aggccgacgc gaagcggcgg ggcgtaggga |
| 14881 | gcgcagcgac cgaagggtag gcgctttttg cagctcttcg gctgtgcgct ggccagacag |
| 14941 | ttatgcacag gccaggcggg ttttaagagt tttaataagt tttaaagagt tttaggcgga |
| 15001 | aaaatcgcct tttttctctt ttatatcagt cacttacatg tgtgaccggt tcccaatgta |
| 15061 | cggctttggg ttcccaatgt acgggttccg gttcccaatg tacggctttg ggttcccaat |
| 15121 | gtacgtgcta tccacaggaa agagaccttt tcgacctttt tcccctgcta gggcaatttg |
| 15181 | ccctagcatc tgctccgtac attaggaacc ggcggatgct tcgccctcga tcaggttgcg |
| 15241 | gtagcgcatg actaggatcg ggccagcctg ccccgcctcc tccttcaaat cgtactccgg |
| 15301 | caggtcattt gacccgatca gcttgcgcac ggtgaaacag aacttcttga actctccggc |
| 15361 | gctgccactg cgttcgtaga tcgtcttgaa caaccatctg gcttctgcct tgcctgcggc |
| 15421 | gcggcgtgcc aggcggtaga gaaaacggcc gatgccggga tcgatcaaaa agtaatcggg |
| 15481 | gtgaaccgtc agcacgtccg ggttcttgcc ttctgtgatc tcgcggtaca tccaatcagc |
| 15541 | tagctcgatc tcgatgtact ccggccgccc ggtttcgctc tttacgatct tgtagcggct |
| 15601 | aatcaaggct tcaccctcgg ataccgtcac caggcggccg ttcttggcct tcttcgtacg |
| 15661 | ctgcatggca acgtgcgtgg tgtttaaccg aatgcaggtt tctaccaggt cgtctttctg |
| 15721 | ctttccgcca tcggctcgcc ggcagaactt gagtacgtcc gcaacgtgtg gacggaacac |
| 15781 | gcggccgggc ttgtctccct tcccttcccg gtatcggttc atggattcgg ttagatggga |
| 15841 | aaccgccatc agtaccaggt cgtaatccca cacactggcc atgccggccg gccctgcgga |
| 15901 | aacctctacg tgcccgtctg gaagctcgta gcggatcacc tcgccagctc gtcggtcacg |
| 15961 | cttcgacaga cggaaaacgg ccacgtccat gatgctgcga ctatcgcggg tgcccacgtc |
| 16021 | atagagcatc ggaacgaaaa aatctggttg ctcgtcgccc ttgggcggct tcctaatcga |
| 16081 | cggcgcaccg gctgccggcg gttgccggga ttctttgcgg attcgatcag cggccgcttg |
| 16141 | ccacgattca ccggggcgtg cttctgcctc gatgcgttgc cgctgggcgg cctgcgcggc |
| 16201 | cttcaacttc tccaccaggt catcacccag cgccgcgccg atttgtaccg ggccggatgg |
| 16261 | tttgcgaccg tcacgccgat tcctcgggct tgggggttcc agtgccattg cagggccggc |
| 16321 | agacaaccca gccgcttacg cctggccaac cgcccgttcc tccacacatg gggcattcca |
| 16381 | cggcgtcggt gcctggttgt tcttgatttt ccatgccgcc tcctttagcc gctaaaattc |
| 16441 | atctactcat ttattcattt gctcatttac tctggtagct gcgcgatgta ttcagatagc |
| 16501 | agctcggtaa tggtcttgcc ttggcgtacc gcgtacatct tcagcttggt gtgatcctcc |
| 16561 | gccggcaact gaaagttgac ccgcttcatg gctggcgtgt ctgccaggct ggccaacgtt |
| 16621 | gcagccttgc tgctgcgtgc gctcggacgg ccggcactta gcgtgtttgt gcttttgctc |
| 16681 | attttctctt tacctcatta actcaaatga gttttgattt aatttcagcg gccagcgcct |
| 16741 | ggacctcgcg ggcagcgtcg ccctcgggtt ctgattcaag aacggttgtg ccggcggcgg |
| 16801 | cagtgcctgg gtagctcacg cgctgcgtga tacgggactc aagaatgggc agctcgtacc |
| 16861 | cggccagcgc ctcggcaacc tcaccgccgt |
| // | |

TABLE 4
SEQ. ID. NO. 47
Plant Nuclease transformation plasmid for transit and stable transformation Nptll TP Nif
HDK101 PGE #66

| | |
|---|---|
| FEATURES | Location/Qualifiers |
| CDS | 12307 . . . 13275 |
| | SPC-AADAR |
| repeat_region | 13727 . . . 14007 |
| | ORI-E1 |
| rep_origin | complement(14817 . . . 15817) |
| | PVS1\rep\AGRO |
| misc_feature | 1730 . . . 1797 |
| | RB |
| misc_feature | complement(11927 . . . 11970) |
| | LB |
| CDS | complement(2544 . . . 3335) |
| | Reporter-NptII |
| Artificial CDS | 4661 . . . 5755 |
| | TPH101 |
| Artificial CDS | 6946 . . . 8607 |
| | TPD101 |
| Artificial CDS | 9785 . . . 11527 |
| | TPK101 |
| promoter | 3771 . . . 4658 |
| | 2X355 |
| promoter | 6052 . . . 6944 |
| | 2X355 |
| promoter | 8891 . . . 9782 |
| | 2X35SP |

```
        1 tgcgcgtgcc tttgatcgcc cgcgacacga caaaggccgc ttgtagcctt ccatccgtga
      61 cctcaatgcg ctgcttaacc agctccacca ggtcggcggt ggcccatatg tcgtaagggc
    121 ttggctgcac cggaatcagc acgaagtcgg ctgccttgat cgcggacaca gccaagtccg
    181 ccgcctgggg cgctccgtcg atcactacga agtcgcgccg gccgatggcc ttcacgtcgc
    241 ggtcaatcgt cgggcggtcg atgccgacaa cggttagcgg ttgatcttcc cgcacggccg
    301 cccaatcgcg ggcactgccc tggggatcgg aatcgactaa cagaacatcg gccccggcga
    361 gttgcagggc gcgggctaga tgggttgcga tggtcgtctt gcctgacccg cctttctggt
    421 taagtacagc gataaccttc atgcgttccc cttgcgtatt tgtttattta ctcatcgcat
    481 catatacgca gcgaccgcat gacgcaagct gttttactca aatacacatc acctttttag
    541 acggcggcgc tcggtttctt cagcggccaa gctggccggc caggccgcca gcttggcatc
    601 agacaaaccg gccaggattt catgcagccg cacggttgag acgtgcgcgg gcggctcgaa
    661 cacgtacccg gccgcgatca tctccgcctc gatctcttcg gtaatgaaaa acggttcgtc
    721 ctggccgtcc tggtgcggtt tcatgcttgt tcctcttggc gttcattctc ggcggccgcc
    781 agggcgtcgg cctcggtcaa tgcgtcctca cggaaggcac cgcgccgcct ggcctcggtg
    841 ggcgtcactt cctcgctgcg ctcaagtgcg cggtacaggg tcgagcgatg cacgccaagc
    901 agtgcagccg cctctttcac ggtgcggcct tcctggtcga tcagctcgcg ggcgtgcgcg
    961 atctgtgccg gggtgagggt agggcggggg ccaaacttca cgcctcgggc cttggcggcc
  1021 tcgcgcccgc tccgggtgcg gtcgatgatt agggaacgct cgaactcggc aatgccggcg
  1081 aacacggtca acaccatgcg gccggccggc gtggtggtgt cggcccacgg ctctgccagg
  1141 ctacgcaggc ccgcgccggc ctcctggatg cgctcggcaa tgtccagtag gtcgcgggtg
  1201 ctgcgggcca ggcggtctag cctggtcact gtcacaacgt cgccagggcg taggtggtca
  1261 agcatcctgg ccagctccgg gcggtcgcgc ctggtgccgg tgatcttctc ggaaaacagc
  1321 ttggtgcagc cggccgcgtg cagttcggcc cgttggttgg tcaagtcctg gtcgtcggtg
  1381 ctgacgcggg catagcccag caggccagcg gcggcgctct tgttcatggc gtaatgtctc
  1441 cggttctagt cgcaagtatt ctactttatg cgactaaaac acgcgacaag aaaacgccag
  1501 gaaaagggca gggcggcagc ctgtcgcgta acttaggact tgtgcgacat gtcgttttca
  1561 gaagacggct gcactgaacg tcagaagccg actgcactat agcagcggag gggttggatc
  1621 aaagtacttt gatcccgagg ggaaccctgt ggttggcatg cacatacaaa tggacgaacg
  1681 gataaacctt ttcacgccct tttaaatatc cgttattcta ataaacgctc ttttctctta
  1741 ggtttacccg ccaatatatc ctgtcaaaca ctgatagttt aaactgaagg cgggaaacga
  1801 caatctgatc caagctcaag ctaagcttca gagccaccac cctcagagcc gccaccagaa
  1861 ccaccaccag agccgccgcc agcattgaca ggaggcccga tctagtaaca tagatgacac
  1921 cgcgcgcgat aatttatcct agtttgcgcg ctatattttg ttttctatcg cgtattaaat
  1981 gtataattgc gggactctaa tcataaaaac ccatctcata aataacgtca tgcattacat
  2041 gttaattatt acatgcttaa cgtaattcaa cagaaattat atgataatca tcgcaagacc
  2101 ggcaacagga ttcaatctta agaaacttta ttgccaaatg tttgaacgat cggggatcat
  2161 ccgggtctgt ggcgggaact ccacgaaaat atccgaacgc agcaagatat cgcggtccat
  2221 ctcggtcttg cctgggcagt cgccgccgac gccgttgatg tggacgccgg gcccgatcat
  2281 attgtcgctc aggatcgtgg cgttgtgctt gtcggccgtt gctgtcgtaa tgatatcggc
  2341 accttcgacc gcctgttccg cagagatccc gtgggcgaag aactccagca tgagatcccc
  2401 gcgctggagg atcatccagc cggcgtcccg gaaaacgatt ccgaagccca acctttcata
  2461 gaaggcggcg gtggaatcga aatctcgtga tggcaggttg ggcgtcgctt ggtcggtcat
  2521 ttcgaacccc agagtcccgc tcagaagaac tcgtcaagaa ggcgatagaa ggcgatgcgc
  2581 tgcgaatcgg gagcggcgat accgtaaagc acgaggaagc ggtcagccca ttcgccgcca
  2641 agctcttcag caatatcacg ggtagccaac gctatgtcct gatagcggtc cgccacaccc
  2701 agccggccac agtcgatgaa tccagaaaag cggccatttt ccaccatgat attcggcaag
  2761 caggcatcgc catgggtcac gacgagatca tcgccgtcgg gcatgcgcgc cttgagcctg
  2821 gcgaacagtt cggctggcgc gagcccctga tgctcttcgt ccagatcatc ctgatcgaca
  2881 agaccggctt ccatccgagt acgtgctcgc tcgatgcgat gtttcgcttg gtggtcgaat
  2941 gggcaggtag ccggatcaag cgtatgcagc cgccgcattg catcagccat gatggatact
  3001 ttctcggcag gagcaaggtg agatgacagg agatcctgcc ccggcacttc gcccaatagc
  3061 agccagtccc ttcccgcttc agtgacaacg tcgagcacag ctgcgcaagg aacgcccgtc
  3121 gtggccagcc acgatagccg cgctgcctcg tcctgcagtt cattcagggc accggacagg
  3181 tcggtcttga caaaaagaac cgggcgcccc tgcgctgaca gccggaacac ggcggcatca
  3241 gagcagccga ttgtctgttg tgcccagtca tagccgaata gcctctccac ccaagcggcc
  3301 ggagaacctg cgtgcaatcc atcttgttca atcatgcgaa acgatccaga tccggtgcag
  3361 attatttgga ttgagagtga atatgagact ctaattggat accgagggga atttatggaa
  3421 cgtcagtgga gcatttttga caagaaatat ttgctagctg atagtgacct taggcgactt
  3481 ttgaacgcgc aataatggtt tctgacgtat gtgcttagct cattaaactc cagaaacccg
  3541 cggctgagtg gctccttcaa cgttgcggtt ctgtcagttc caaacgtaaa acggcttgtc
  3601 ccgcgtcatc ggcgggggtc ataacgtgac tcccttaatt ctccgctcat gatcagattg
  3661 tcgtttcccg ccttcaggta ccgcgatcgc tcgcgacctg caggcataaa gccgtcagtg
  3721 tccgcataaa gaaccaaccc ataataccca taatagctgt ttgccaaccg gtcaacatgt
  3781 ggagcacgac acacttgtct actccaaaaa tatcaaagat acagtctcag aagaccaaag
  3841 ggcaattgag acttttcaac aaagggtaat atccggaaac ctcctcggat tccattgccc
  3901 agctatctgt cactttattg tgaagatagt ggaaaaggaa ggtggctcct acaaatgcca
  3961 tcattgcgat aaaggaaagg ccatcgttga agatgcctct gccgacagtg gtcccaaaga
  4021 tggaccccca cccacgagga gcatcgtgga aaaagaagac gttccaacca cgtcttcaaa
  4081 gcaagtggat tgatgtgata acatggtgga gcacgacaca cttgtctact ccaaaaatat
  4141 caaagataca gtctcagaag accaaagggc aattgagact tttcaacaaa gggtaatatc
  4201 cggaaacctc ctcggattcc attgcccagc tatctgtcac tttattgtga agatagtgga
  4261 aaaggaaggt ggctcctaca aatgccatca ttgcgataaa ggaaaggcca tcgttgaaga
  4321 tgcctctgcc gacagtggtc ccaaagatgg acccccaccc acgaggagca tcgtggaaaa
  4381 agaagacgtt ccaaccacgt cttcaaagca agtggattga tgtgatatct ccactgacgt
  4441 aagggatgac gcacaatccc actatccttc gcaagaccct tcctctatat aaggaagttc
  4501 atttcatttg gagaggacgt cgagagttct caacacaaca tatacaaaac aaacgaatct
  4561 caagcaatca agcattctac ttctattgca gcaatttaaa tcatttcttt taaagcaaaa
  4621 gcaattttct gaaaattttc accatttacg aacgatagcc atggctagcg ctaccggact
  4681 cagatctatg gcttcctctg tcatttcttc agcagctgtt gccacacgca gcaatgttac
  4741 acaagctagc atggttgcac ctttcactgg tctcaaatct tcagccactt tccctgttac
  4801 aaagaagcaa aaccttgaca tcacttccat tgctagcaat ggtggaagag ttagctgcag
  4861 agctcaaatg agacaaattg ctttttatgg aaaaggagga attggaaaat ctactacttc
  4921 tcaaaatact ttagctgcta tggctaatag acatggacaa agaattatga ttgtaggatg
  4981 tgatcctaaa gctgattcta ctagattaat tttaaatgct aaagctcaaa ctactgtatt
  5041 acatgtagct gctgaaagag gagctgtaga agatgtagaa ttagatgaag tattaaaacc
  5101 tggatttgga ggaattaaat gtgtagaatc tggaggacct gaacctggag taggatgtgc
  5161 tggaagagga attattactg ctattaattt tttagaagaa gaaggagctt atactgattt
  5221 agattttgta tcttatgatg tattaggaga tgtagtatgt ggaggatttg ctatgcctat
  5281 tagagaaaat aaagctcaag aaatttatat tgtatgttct ggagaaatga tggctatgta
  5341 tgctgctaat aatattgcta gaggagtatt aaaatatgct catgctggag gagtaagatt
  5401 aggaggatta atttgtaatt ctagaaaagt agatagagaa actgaattaa ttgaaaattt
  5461 agctgctaga ttaaatactc aaatgattca ttttgtacct agagataatg tagtacaaag
  5521 agctgaaatt agaagaatga ctgtagaaca atatgctcct gaagataatc aagctcaaga
  5581 atatgatcaa ttagctcaaa aaattattaa taatgaaaaa ttaactattc ctactccttt
  5641 agaaatggat gaattagaag aattattaat tgaatttgga ttattaggag atgaagaaga
  5701 tagacaaaaa caaattgctg ctcaagatgc tgctattaaa gctactgctg ctaaataagg
  5761 atccacctga tctagagtcc gcaaaaatca ccagtctctc tctacaaatc tatctctctc
  5821 tatttttctc cagaataatg tgtgagtagt tcccagataa gggaattagg gttcttatag
  5881 ggtttcgctc atgtgttgag catataagaa acccttagta tgtatttgta tttgtaaaat
  5941 acttctatca ataaaatttc taattcctaa aaccaaaatc cagtgacgcg gccgcaccca
  6001 taatacccat aatagctgtt tgccatcgct accttaggac cgttatagtt aaccggtcaa
  6061 catgtggagc acgacacact tgtctactcc aaaaatatca aagatacagt ctcagaagac
  6121 caaagggcaa ttgagacttt tcaacaaagg gtaatatccg gaaacctcct cggattccat
  6181 tgcccagcta tctgtcactt tattgtgaag atagtggaaa aggaaggtgg ctcctacaaa
  6241 tgccatcatt gcgataaagg aaaggccatc gttgaagatg cctctgccga cagtggtccc
  6301 aaagatggac ccccacccac gaggagcatc gtggaaaaag aagacgttcc aaccacgtct
  6361 tcaaagcaag tggattgatg tgataacatg gtggagcacg acacacttgt ctactccaaa
  6421 aatatcaaag atacagtctc agaagaccaa agggcaattg agacttttca acaaagggta
  6481 atatccggaa acctcctcgg attccattgc ccagctatct gtcactttat tgtgaagata
  6541 gtggaaaagg aaggtggctc ctacaaatgc catcattgcg ataaaggaaa ggccatcgtt
  6601 gaagatgcct ctgccgacag tggtcccaaa gatggacccc cacccacgag gagcatcgtg
  6661 gaaaaagaag acgttccaac cacgtcttca aagcaagtgg attgatgtga tatctccact
  6721 gacgtaaggg atgacgcaca atcccactat ccttcgcaag acccttcctc tatataagga
  6781 agttcatttc atttggagag gacgtcgaga gttctcaaca caacatatac aaaacaaacg
  6841 aatctcaagc aatcaagcat tctacttcta ttgcagcaat ttaaatcatt tcttttaaag
  6901 caaaagcaat tttctgaaaa ttttcaccat ttacgaacga tagccatggc tagcgctacc
  6961 ggactcagat ctatggcttc ctctgtcatt tcttcagcag ctgttgccac acgcagcaat
  7021 gttacacaag ctagcatggt tgcacctttc actggtctca aatcttcagc cactttccct
  7081 gttacaaaga agcaaaacct tgacatcact tccattgcta gcaatggtgg aagagttagc
  7141 tgcagagctc aaatggcttc tgaaaaaatt gaacaaaata aacaattaat tcaagaagta
  7201 ttagatgctt atcctgctaa agctgctaaa agaagaaaaa aacatttaaa tgtaattgaa
  7261 gaaaaaggag ctgattgtgg agtaaaatct aatgtaaaat ctgtacctgg agtaatgact
  7321 actagaggat gtgcttttgc tggagctaaa ggagtagtat ggggacctgt aaaagatatg
  7381 gtacatattt ctcatggacc tgtaggatgt ggatattatt cttgggctgg aagaagaaat
  7441 tattataatg gagtaactgg agtagatact tttggaacta tgcaatttac ttctgatttt
  7501 caagaaagag atattgtatt tggaggagat aaaaaattag ctaaaattat gaatgaaatt
  7561 gaagaattat ttcctttaaa tgctggaatt actattgaat ctgaatgtcc tgtaggatta
  7621 attggagatg atattgaagc tgtagctaaa aaagcttcta aagaattaaa taaacctgta
  7681 gtacctgtaa gatgtgaagg atttagagga gtatctcaat ctttaggaca tcatattgct
  7741 aatgatactg taagagattg ggtatatgaa ccttctgcta aagtaactaa tgaagaaatt
  7801 ggatttgaaa aaactcctta tgatgtatct ttaattgctg attataatat tggaggagat
  7861 ggatggtctt ctagattatt attagatgaa attggattaa gagtagtatc tcaagctact
  7921 ggagatggaa cttataatga agtatttatg gctcctagag taaatttaaa tttaattcat
  7981 tgttatagat ctatgaatta tatttgtaga tatatggaag aagaatatgg aattccttgg
  8041 gtagaattta atttttttgg accttctcaa attgctaaat ctttaagaaa aattgcttct
  8101 ttttttgatg ataaaattaa agaaaatact gaaaaagtaa ttgctagata tcaagaacaa
  8161 gctgatgctg taattgctaa atatagacct agattagaag gaaaaaaagt aatgatgatg
  8221 gtaggaggat taagacctag acatattatt cctgcttttg atgatttagg aatggaagta
  8281 attggaactg gatatgaatt tggacatgga gatgattata aaagaactgc tgattatgct
  8341 caagaaggaa ctttaattta tgatgatgta tctggatatg aatttgaaga atttgctaaa
  8401 aaattaaaac ctgatttaat tgcttctgga attaaagaaa aatatgtatt tcaaaaaatg
  8461 ggaatgcctt ttagacaaat gcattcttgg gattattctg gaccttatca tggatatgat
  8521 ggatttgcta tttttgctag agatatggat ttagctttaa attctcctac ttggaattta
  8581 attaaagctc cttggaaaca agctaaatag gatccacctg atctagagtc cgcaaaaatc
  8641 accagtctct ctctacaaat ctatctctct ctatttttct ccagaataat gtgtgagtag
  8701 ttcccagata agggaattag ggttcttata gggtttcgct catgtgttga gcatataaga
  8761 aacccttagt atgtatttgt atttgtaaaa tacttctatc aataaaattt ctaattccta
  8821 aaaccaaaat ccagtgacgc ggccgctcgc taccttagga ccgttatagt taattaccct
  8881 gttatcccta accggtcaac atgtggagca cgacacactt gtctactcca aaaatatcaa
  8941 agatacagtc tcagaagacc aaagggcaat tgagactttt caacaaaggg taatatccgg
  9001 aaacctcctc ggattccatt gcccagctat ctgtcacttt attgtgaaga tagtggaaaa
  9061 ggaaggtggc tcctacaaat gccatcattg cgataaagga aaggccatcg ttgaagatgc
  9121 ctctgccgac agtggtccca aagatggacc cccacccacg aggagcatcg tggaaaaaga
  9181 agacgttcca accacgtctt caaagcaagt ggattgatgt gataacatgg tggagcacga
  9241 cacacttgtc tactccaaaa atatcaaaga tacagtctca gaagaccaaa gggcaattga
  9301 gacttttcaa caaagggtaa tatccggaaa cctcctcgga ttccattgcc cagctatctg
  9361 tcactttatt gtgaagatag tggaaaagga aggtggctcc tacaaatgcc atcattgcga
  9421 taaaggaaag gccatcgttg aagatgcctc tgccgacagt ggtcccaaag atggaccccc
  9481 acccacgagg agcatcgtgg aaaaagaaga cgttccaacc acgtcttcaa agcaagtgga
  9541 ttgatgtgat atctccactg acgtaaggga tgacgcacaa tcccactatc cttcgcaaga
  9601 cccttcctct atataaggaa gttcatttca tttggagagg acgtcgagag ttctcaacac
  9661 aacatataca aaacaaacga atctcaagca atcaagcatt ctacttctat tgcagcaatt
  9721 taaatcattt cttttaaagc aaaagcaatt ttctgaaaat tttcaccatt tacgaacgat
  9781 agccatggct agcgctaccg gactcagatc tatggcttcc tctgtcattt cttcagcagc
  9841 tgttgccaca cgcagcaatg ttacacaagc tagcatggtt gcacctttca ctggtctcaa
  9901 atcttcagcc actttccctg ttacaaagaa gcaaaacctt gacatcactt ccattgctag
  9961 caatggtgga agagttagct gcagagctca aatgtctcaa aatgtagata aaattaaaga
10021 tcattttcaa ttatttcaag aacctgaata tcaagaaatg tttgctagaa aaagagaatt
10081 tgaaggagga gcttctaaag aagaaattga aagagtaaga gaatggacta aatcttggga
10141 atatagagaa aaaaattttg ctagagaagc tttaactatt aatcctgcta aagcttgtca
10201 acctttagga gctatttttg ctgctgctgg atttgaagga actttacctt ttgtacatgg
10261 atctcaagga tgtgtagctt attttagatc tcatttaact agaaattata aagaaccttt
10321 tcaagctgta tcttcttcta tgactgaaga tgctgctgta tttggaggat taaaaaatat
10381 gattgatgga ttagctaatt cttatgcttt atataaacct aaaatgattg ctttatgtac
10441 tacttgtatg gctgaagtaa ttggagatga tttaggatct tttattacta attctaaaaa
10501 tgaaggagct gtacctcaag attttcctgt accttttgct catactcctt cttttgtagg
10561 atctcatatt actggatatg ataatatgtt aaaaggaatt ttaattgctt taactgatgg
10621 aaaaaaaact gaaactgata atggaaaaat taattttatt cctggatttg atccttatat
10681 tggaaatatt agagatttaa aaaatatttt atctttaatg gatgtacctt ctactgtatt
10741 agctgataat gctgaatctt ttgattctcc taatttagga gaatttaaaa tgtataatgg
10801 aggaactact ttagaagaag ctggagattc tattaatgct aaagctacta tttcttttca
10861 aaaatattct actcctaaaa ctttagaata tttaaaacaa gaaggaggac aaaaaactgc
10921 tacttataga cctattggag taagaggaac tgatgaattt ttaatggctt tatctgaatt
10981 aactggaaaa gctattcctg aagaattaga aattgaaaga ggaagagtag tagatgctat
11041 tactgattct caagcttggt tacatggaaa aagaattgct atttatggag atcctgatca
11101 tgtattagga ttattaaatt ttactttaga attaggaatg caacctgtac atgtagtagt
11161 aaataatgga aatgtagctg gatttgaaga agaagctaaa gaattattag ctaatgatcc
11221 taatggaaaa gaagctactg tatggattgg aaaagattta tggcatttaa gatctttatt
11281 agatactgaa cctgtagatt tattaattgg aaattcttat ggaaaatttt tacaaagaga
11341 tactggaact cctttagtaa gaattggata tcctattttt gatagacatc atcaacatag
11401 atattctatt ttaggatata aaggagcttt taatttaatt aattggattg taaatactat
11461 tttagatgaa ttagatagag gatctatgga tttaggagta aatgatactt cttttgattt
11521 agtaagataa ggatccacct gatctagagt ccgcaaaaat caccagtctc tctctacaaa
11581 tctatctctc tctatttttc tccagaataa tgtgtgagta gttcccagat aagggaatta
11641 gggttcttat agggtttcgc tcatgtgttg agcatataag aaacccttag tatgtatttg
11701 tatttgtaaa atacttctat caataaaatt tctaattcct aaaaccaaaa tccagtgacg
11761 cggccgcatt accctgttat ccctattaat taagagctcg ctaccttaag agaggatatc
11821 ggcgcgcctc tagaatttaa atggatccta cgtactcgag gaattcaatt cggcgttaat
11881 tcagtacatt aaaaacgtcc gcaatgtgtt attaagttgt ctaagcgtca atttgtttac
11941 accacaatat atcctgccac cagccagcca acagctcccc gaccggcagc tcggcacaaa
12001 atcaccactc gatacaggca gcccatcagt ccgggacggc gtcagcggga gagccgttgt
12061 aaggcggcag actttgctca tgttaccgat gctattcgga agaacggcaa ctaagctgcc
12121 gggtttgaaa cacggatgat ctcgcggagg gtagcatgtt gattgtaacg atgacagagc
12181 gttgctgcct gtgatcaatt cgggcacgaa cccagtggac ataagcctcg ttcggttcgt
12241 aagctgtaat gcaagtagcg taactgccgt cacgcaactg gtccagaacc ttgaccgaac
12301 gcagcggtgg taacggcgca gtggcggttt tcatggcttc ttgttatgac atgttttttt
12361 ggggtacagt ctatgcctcg ggcatccaag cagcaagcgc gttacgccgt gggtcgatgt
12421 ttgatgttat ggagcagcaa cgatgttacg cagcagggca gtcgccctaa aacaaagtta
12481 aacatcatgg gggaagcggt gatcgccgaa gtatcgactc aactatcaga ggtagttggc
12541 gtcatcgagc gccatctcga accgacgttg ctggccgtac atttgtacgg ctccgcagtg
12601 gatggcggcc tgaagccaca cagtgatatt gatttgctgg ttacggtgac cgtaaggctt
12661 gatgaaacaa cgcggcgagc tttgatcaac gaccttttgg aaacttcggc ttcccctgga
12721 gagagcgaga ttctccgcgc tgtagaagtc accattgttg tgcacgacga catcattccg
12781 tggcgttatc cagctaagcg cgaactgcaa tttggagaat ggcagcgcaa tgacattctt
12841 gcaggtatct tcgagccagc cacgatcgac attgatctgg ctatcttgct gacaaaagca
12901 agagaacata gcgttgcctt ggtaggtcca gcggcggagg aactctttga tccggttcct
12961 gaacaggatc tatttgaggc gctaaatgaa accttaacgc tatggaactc gccgcccgac
13021 tgggctggcg atgagcgaaa tgtagtgctt acgttgtccc gcatttggta cagcgcagta
13081 accggcaaaa tcgcgccgaa ggatgtcgct gccgactggg caatggagcg cctgccggcc
13141 cagtatcagc ccgtcatact tgaagctaga caggcttatc ttggacaaga agaagatcgc
13201 ttggcctcgc gcgcagatca gttggaagaa tttgtccact acgtgaaagg cgagatcacc
13261 aaggtagtcg gcaaataatg tctagctaga aattcgttca agccgacgcc gcttcgccgg
13321 cgttaactca agcgattaga tgcactaagc acataattgc tcacagccaa actatcaggt
13381 caagtctgct tttattattt ttaagcgtgc ataataagcc ctacacaaat tgggagatat
13441 atcatgcatg accaaaatcc cttaacgtga gttttcgttc cactgagcgt cagaccccgt
13501 agaaaagatc aaaggatctt cttgagatcc tttttttctg cgcgtaatct gctgcttgca
13561 aacaaaaaaa ccaccgctac cagcggtggt ttgtttgccg gatcaagagc taccaactct
13621 ttttccgaag gtaactggct tcagcagagc gcagatacca aatactgtcc ttctagtgta
13681 gccgtagtta ggccaccact tcaagaactc tgtagcaccg cctacatacc tcgctctgct
13741 aatcctgtta ccagtggctg ctgccagtgg cgataagtcg tgtcttaccg ggttggactc
13801 aagacgatag ttaccggata aggcgcagcg gtcgggctga acggggggtt cgtgcacaca
13861 gcccagcttg gagcgaacga cctacaccga actgagatac ctacagcgtg agctatgaga
13921 aagcgccacg cttcccgaag ggagaaaggc ggacaggtat ccggtaagcg gcagggtcgg
13981 aacaggagag cgcacgaggg agcttccagg gggaaacgcc tggtatcttt atagtcctgt
14041 cgggtttcgc cacctctgac ttgagcgtcg atttttgtga tgctcgtcag gggggcggag
14101 cctatggaaa aacgccagca acgcggcctt tttacggttc ctggcctttt gctggccttt
14161 tgctcacatg ttctttcctg cgttatcccc tgattctgtg gataaccgta ttaccgcctt
14221 tgagtgagct gataccgctc gccgcagccg aacgaccgag cgcagcgagt cagtgagcga
14281 ggaagcggaa gagcgcctga tgcggtattt tctccttacg catctgtgcg gtatttcaca
14341 ccgcatatgg tgcactctca gtacaatctg ctctgatgcc gcatagttaa gccagtatac
14401 actccgctat cgctacgtga ctgggtcatg gctgcgcccc gacacccgcc aacacccgct
14461 gacgcgccct gacgggcttg tctgctcccg gcatccgctt acagacaagc tgtgaccgtc
14521 tccgggagct gcatgtgtca gaggttttca ccgtcatcac cgaaacgcgc gaggcagggt
14581 gccttgatgt gggcgccggc ggtcgagtgg cgacggcgcg gcttgtccgc gccctggtag
14641 attgcctggc cgtaggccag ccatttttga gcggccagcg gccgcgatag gccgacgcga
14701 agcggcgggg cgtagggagc gcagcgaccg aagggtaggc gctttttgca gctcttcggc
14761 tgtgcgctgg ccagacagtt atgcacaggc caggcgggtt ttaagagttt taataagttt
14821 taaagagttt taggcggaaa aatcgccttt tttctctttt atatcagtca cttacatgtg
14881 tgaccggttc ccaatgtacg gctttgggtt cccaatgtac gggttccggt tcccaatgta
14941 cggctttggg ttcccaatgt acgtgctatc cacaggaaag agaccttttc gacctttttc
15001 ccctgctagg gcaatttgcc ctagcatctg ctccgtacat taggaaccgg cggatgcttc
15061 gccctcgatc aggttgcggt agcgcatgac taggatcggg ccagcctgcc ccgcctcctc
15121 cttcaaatcg tactccggca ggtcatttga cccgatcagc ttgcgcacgg tgaaacagaa
15181 cttcttgaac tctccggcgc tgccactgcg ttcgtagatc gtcttgaaca accatctggc
15241 ttctgccttg cctgcggcgc ggcgtgccag gcggtagaga aaacggccga tgccgggatc
15301 gatcaaaaag taatcggggt gaaccgtcag cacgtccggg ttcttgcctt ctgtgatctc
15361 gcggtacatc caatcagcta gctcgatctc gatgtactcc ggccgcccgg tttcgctctt
15421 tacgatcttg tagcggctaa tcaaggcttc accctcggat accgtcacca ggcggccgtt
15481 cttggccttc ttcgtacgct gcatggcaac gtgcgtggtg tttaaccgaa tgcaggtttc
15541 taccaggtcg tctttctgct ttccgccatc ggctcgccgg cagaacttga gtacgtccgc
15601 aacgtgtgga cggaacacgc ggccgggctt gtctcccttc ccttcccggt atcggttcat
15661 ggattcggtt agatgggaaa ccgccatcag taccaggtcg taatcccaca cactggccat
15721 gccggccggc cctgcggaaa cctctacgtg cccgtctgga agctcgtagc ggatcacctc
15781 gccagctcgt cggtcacgct tcgacagacg gaaaacggcc acgtccatga tgctgcgact
15841 atcgcgggtg cccacgtcat agagcatcgg aacgaaaaaa tctggttgct cgtcgccctt
15901 gggcggcttc ctaatcgacg gcgcaccggc tgccggcggt tgccgggatt ctttgcggat
15961 tcgatcagcg gccgcttgcc acgattcacc ggggcgtgct tctgcctcga tgcgttgccg
16021 ctgggcggcc tgcgcggcct tcaacttctc caccaggtca tcacccagcg ccgcgccgat
16081 ttgtaccggg ccggatggtt tgcgaccgtc acgccgattc ctcgggcttg ggggttccag
16141 tgccattgca gggccggcag acaacccagc cgcttacgcc tggccaaccg cccgttcctc
16201 cacacatggg gcattccacg gcgtcggtgc ctggttgttc ttgattttcc atgccgcctc
16261 ctttagccgc taaaattcat ctactcattt attcatttgc tcatttactc tggtagctgc
16321 gcgatgtatt cagatagcag ctcggtaatg gtcttgcctt ggcgtaccgc gtacatcttc
16381 agcttggtgt gatcctccgc cggcaactga aagttgaccc gcttcatggc tggcgtgtct
16441 gccaggctgg ccaacgttgc agccttgctg ctgcgtgcgc tcggacggcc ggcacttagc
16501 gtgtttgtgc ttttgctcat tttctcttta cctcattaac tcaaatgagt tttgatttaa
16561 tttcagcggc cagcgcctgg acctcgcggg cagcgtcgcc ctcgggttct gattcaagaa
16621 cggttgtgcc ggcggcggca gtgcctgggt agctcacgcg ctgcgtgata cgggactcaa
16681 gaatgggcag ctcgtacccg gccagcgcct cggcaacctc accgccgt
```

TABLE 5
SEQ. ID. NO. 48

| | |
|---|---|
| PGE #0148 | 12181 bp DNA circular |
| ORGANISM | E. Coli/bombardment into plant. |
| COMMENT | pBS/PUC |
| FEATURES | Location/Qualifiers |
| CDS | complement(11196 . . . 12053) |
| | AMPR |
| rep_origin | complement(10419 . . . 11033) |
| | ORIPUC |
| misc_feature | complement(8485 . . . 9974) |
| | Tom\Chl\2 |
| misc_feature | 659 . . . 2582 |
| | Tom\CHL\1 |
| CDS | complement(8675 . . . 8745) |
| | tRNA-G |
| CDS | 2495 . . . 2568 |
| | tRNA-fM |
| terminator | 8220 . . . 8414 |
| | PsbA\trminator |
| promoter | 2644 . . . 2836 |
| | rrn16P |
| CDS | 6941 . . . 7870 |
| | aadA |
| RBS | 3734 . . . 3813 |
| | Artificial\SD |
| RBS | 5312 . . . 5327 |
| | Artificial\SD |
| RBS | 6871 . . . 6939 |
| | Artificial\SD |
| CDS | 2837 . . . 3727 |
| | Artificial\NifH\912 |
| CDS | 3815 . . . 5305 |
| | Artificial\NifD912 |
| CDS | 5328 . . . 6863 |
| | Artificial\NifK\912 |

```
        1 ctgaaattgt aagcgttaat attttgttaa aattcgcgtt aaatttttgt taaatcagct
      61 cattttttaa ccaataggcc gaaatcggca aaatccctta taaatcaaaa gaatagaccg
    121 agatagggtt gagtgttgtt ccagtttgga acaagagtcc actattaaag aacgtggact
    181 ccaacgtcaa agggcgaaaa accgtctatc agggcgatgg cccactacgt gaaccatcac
    241 cctaatcaag ttttttgggg tcgaggtgcc gtaaagcact aaatcggaac cctaaaggga
    301 gcccccgatt tagagcttga cggggaaagc cggcgaacgt ggcgagaaag gaagggaaga
    361 aagcgaaagg agcgggcgct agggcgctgg caagtgtagc ggtcacgctg cgcgtaacca
    421 ccacacccgc cgcgcttaat gcgccgctac agggcgcgtc cattcgccat tcaggctgcg
    481 caactgttgg gaagggcgat cggtgcgggc ctcttcgcta ttacgccagc tggcgaaagg
    541 gggatgtgct gcaaggcgat taagttgggt aacgccaggg ttttcccagt cacgacgttg
    601 taaaacgacg gccagtgagc gcgcgtaata cgactcacta tagggcgaat tgggtacccg
    661 attggggcgt ggacataagg gtctttatga cacaatcaac aattcgcttc attttcaatt
    721 aggccttgct ctagcttctt taggggttat tacttctttg gtagctcaac acatgtactc
    781 tttacctgct tatgcattca tagcacaaga ctttactact caagctgcat tatataccca
    841 ccaccaatat atcgcaggat tcatcatgac aggagctttt gctcatggag ctatattttt
    901 cattagagat tacaatccgg agcaaaatga agataatgta ttggcaagaa tgttagatca
    961 taaagaagct atcatatctc atttaagttg ggccagcctc tttctgggat tccataccct
  1021 gggactttat gttcataatg atgtcatgct tgcctttggc actccggaga agcaaatctt
  1081 gattgaaccg atatttgctc aatggataca atccgctcat ggtaaaactt catatgggtt
  1141 cgatgtactt ttatcttcaa cgactggccc agcattcaat gcgggtcgaa gcatctggtt
  1201 gccgggttgg ttaaatgctg ttaatgaaaa tagtaattca ttattcttaa caataggtcc
  1261 tggagacttt ttggttcatc atgctattgc ccttggttta catacaacta cattgatctt
  1321 agtaaaaggt gctttagatg cacgtggttc caagttaatg ccagataaaa aggatttcgg
  1381 ttatagtttt ccgtgcgatg gcccaggacg aggcggtact tgtgatattt cggcatggga
  1441 cgcgttttat ttggcagttt tttggatgtt aaatactatt ggatgggtta ctttttattg
  1501 gcattggaag cacatcacat tatggcaagg taacgtttca cagtttaatg aatcttccac
  1561 ttatttgatg ggctggttaa gggattattt atggttaaac tcttcacaac ttatcaatgg
  1621 atataaccct tttggtatga atagtttatc ggtttgggca tggatgttct tatttggaca
  1681 tcttgtttgg gctactggat ttatgttctt aatttcttgg cgtggatatt ggcaggaatt
  1741 gattgaaact ttagcatggg ctcacgaacg cacacctttg gccaatttga ttcgatggag
  1801 agataaacca gtggcccttt ctattgtaca agcaagattg gttggattag ctcacttttc
  1861 tgtaggttat atattcactt atgcggcttt cttgattgcc tctacgtcgg gcaaatttgg
  1921 ttaattaatg tgtgtattcg cgataatctc atttctttcg acggagaagg gggtccacct
  1981 tcttctattt ctacatctag gattcgactt gtatcatgga tactaatagg aattcaacca
  2041 ttatggcaag gaaaagtttg attcagaggg agaagaagag gcaaaaattg gaacagaaat
  2101 atcattcgat tcgtcgatcc tcaaaaaaag aaataagcaa ggttccgtcg ttgagtgaca
  2161 aatgggaaat ttatggaaag ttacaatccc taccacggaa tagtgcacct acacgccttc
  2221 atcgacgttg ttttttgacc ggaaggccga gagctaacta tcgagacttt ggcctatccg
  2281 gacacatact tcgtgaaatg gttcatgcat gtttgttgcc aggagcaaca agatcaagtt
  2341 ggtaaggatt aacgcttcat ttctatttct atggtcgatg atcatagaag cccctttacc
  2401 attctgtata aatgggctat tctatttgta cagatagggt ggaggggcgc atttaatcct
  2461 tgtttatcta ttagttttca gttcttatct ttggcgcggg gtagagcagt ttggtagctc
  2521 gcaaggctca taaccttgag gtcacgggtt caaatcctgt ctccgcaaca tcttattctg
  2581 gtctcgaggt ccgcataaag aaccacccat aatacccata atagctgttt gccaaccggt
  2641 cgccgtcgtt caatgagaat ggataagagg ctcgtgggat tgacgtgagg gggcagggat
  2701 gactatattt ctgggagcga actccgggcg aatatgaagc gcatggatac aagttatgcc
  2761 ttggaatgaa agacaattcc gaatccgctt tgtctacgaa caaggaagct ataagtaatg
  2821 caactatgaa ggatctatgt ctatcgataa gaaaatccgt caaatcgcat tttacggaaa
  2881 aggcggtatc ggaaaatcca ctacctcaca aaacactctt gctgcaatgg ctgaaatggg
  2941 tcaaagaatc cttatcgttg gatgcgatcc taaagcagac agtacccgcc ttatgcttca
  3001 ctctaaagct caaaccaccg tacttcatct tgcagctgaa cgtggtgctg ttgaagatct
  3061 tgaattagag gaagtaatgt taactggttt tagaggagtt aaatgcgtag agtctggtgg
  3121 tccagaacca ggtgttggat gtgcaggtcg aggaatcatc actgctatca atttcttaga
  3181 ggaaaacggt gcatatcaag atgtagattt tgtttcatac gatgttttag gtgatgtagt
  3241 ttgtggtgga ttcgctatgc cgatccgtga gaacaaagca caagaaatct atatcgtaac
  3301 cagtggtgag atgatggcta tgtacgcagc taataacatc gctagaggta tcttaaagta
  3361 tgcacatact ggaggggttc gattaggcgg gttaatctgt aattcacgta acgtagatcg
  3421 tgaaatcgag ttgatcgaaa ctttggctaa aagattgaat actcaaatga tccattacgt
  3481 tcctcgcgat aacatcgtac aacatgccga gttgcgtcgc atgacagtaa atgaatatgc
  3541 ccctgattcc aatcagtcta atgagtatcg tatcttggcg aataaaatca tcaataatga
  3601 aaatttgaaa gtacctactc ctatcgagat ggaagaacta gaagaactac tgatcgaatt
  3661 tggtatcctg gaatctgaag aaaatgctgc taaaatgatc gggactcctg ctcagtcttc
  3721 cactaaataa tagcggccgc aacccataat acccataata gctgtttgcc atcgctacct
  3781 taggaccgtt atagttaacc ggtggaggca gactatgact cctccagaaa atcaaaacct
  3841 agtagatgag aataaagagt taatcaaaga agtgcttcaa gcttaccctg aaaaggcacg
  3901 aaaaaaaaga gagaagcatt taaatgttca cgaagaaagt aaatccgatt gtggtgtaaa
  3961 atctaacata aagtccattc caggcgttat gactgctcgt gggtgcgcat atgctggtag
  4021 caaaggggta gtttggggtc ctatcaaaga tatgattcat atatcccacg ggcctgtagg
  4081 ttgtgggtac tggtcttggt ccggaagacg taattattac gttggtatca ccggaattaa
  4141 ctctttcggt actatgcatt ttacttccga tttccaggaa agagacatcg tatttggagg
  4201 tgataagaaa ttgaccaaaa taattgatga gttagaaatc ctttttccat taaatcgtgg
  4261 agtgtctatt caatcagaat gccctatcgg tttgattgga ggtgatatcg aagcagttgc
  4321 taaaaaagca tctaagcagt atggaaaacc agtagttcct ttaagatgtg agggtctgcg
  4381 tggagtatca caatctttag gtcatcacat tgctaacgac gcaatcagag attggatttt
  4441 cccagaattt gataaggcta aaaaaaataa tactatcgac tttgaacctt caccgtacga
  4501 tgttgcactt attggagatc ataacatcgg tggagatgct tgggcatctc gtatgttatt
  4561 ggaggaaatg ggtctgagag tagttgctca atggtcagga gatggtactt taaatgaact
  4621 tattcaagga cctgcagcta aattggtact gattcactgc tatcgttcta tgaattacat
  4681 ctgtcgctca ttggaggaac aatatggtat gccgtggatg gaattcaact tttttggacc
  4741 tactaaaatt gcagcttctc ttcgtgagat cgcagctaaa ttcgacagta aaattcaaga
  4801 aaatgcagaa aaagttatcg ctaaatacac cccggtaatg aatgcagtgc ttgagaaata
  4861 tcgccctcgt ttggaaggta acactgttat gctttacgta gggggtcttc gccctcgtca
  4921 tgttgtacct gctttcgaag atttggggat taaagttgta ggtaccgggt atgaatttgc
  4981 acataatgat gattacaaac gcactacaca ctatatcgat aacgctacta ttatctacga
  5041 tgatgttaca gcctatgagt tcgaagaatt tgtaaaagct aaaaaacctg atcttattgc
  5101 ctctggtatc aaagaaaaat acgcttttca gaaaatgggt ctaccttttc gacaaatgca
  5161 tagttgggat tattctggtc cttatcatgg ttatgatcgt tttgccatct ttgctcgaga
  5221 tatggatctt gccctaaata gtcctacttg gagtctaatc ggtgcgcctt ggaaaaaagc
  5281 ggctactaaa gcgaaagctg ctgcttaata actcgagaga tacaacaatg cctcaaaatc
  5341 cagagaaaat tgtagatcat gtacaattat ttcagcaacc tgaatatcaa gaacttttca
  5401 aaaataaaca cgaacaattt gagggagctc attccccaga agaagtacag agagtatccg
  5461 aatggactaa aggttgggag tacagagaaa agaactttgc acgtcaagct ttaacagtaa
  5521 atcctgcaaa aggatgccaa ccagtaggtg ctatcttcgc agctgtagga tttgaaggta
  5581 ctctaccttt cgtacaagga tcccagggtt gtgtagcata ttttagaaca catttatccc
  5641 gtcactacaa agaaccattc tctgctgtat cctcatctat gactgaggat gcagctgtat
  5701 ttggaggtct taacaatatg attgaaggat tacaagtatc atatcaacta tacaaaccta
  5761 aaatgatcgc agtatgtaca acttgtatgg ctgaagtaat tggtgatgat ttaggagcat
  5821 tcatcacaaa tgctaaaaac gcaggttcta ttccacaaga ttttcctgta ccattcgctc
  5881 atactccttc atttgtagga tctcatacaa ccggttatga taatatgatg aagggaatcc
  5941 tttcaaattt aactgagggt aaaaaaaaag ataagaccaa cggaaaaatt aatttcatcc
  6001 caggttttga tacttacgta ggaaataaca gagaactgaa acgtatgtta ggtcttatgg
  6061 gaattgatta taccatctta gcagataatt ctgattacct ggatgctcct aatactggtg
  6121 aatatgatat gtacccgggc ggtaccaaat tagaggatgc agctgattca tgcaacgcag
  6181 aagctactgt agcccttcag agatatacca ctaccaagac tcgagaatac attgagacca
  6241 aatggaaaca acaaactaaa gtattacgtc ctttcggggt aaaaggtacc gacgaatttc
  6301 ttatgacttt atctgaaatg accgggaaac cgatccctca agaacttgag gatgaacgag
  6361 gtcgtttagt agacgctatg actgatagtt atgcctggat tcacgggaaa aaattcgcta
  6421 tctacggtga tccggacctt atttattctg taaccagttt tttacttgaa ttaggggccg
  6481 aacctgtaca tatcctttgt aataatggtg atgaagagtt caaaaaagag atggaagcta
  6541 ttttatctgc gagtccgttt gggaaacaag ctactgtatg gatccagaaa gatttgtggc
  6601 atcttcgatc tttgcttttt actgaacctg tagatttttt tgtaggtaac agttacggga
  6661 aatatttgtg gcgcgacacc aaaattccta tggtacgtat cggttatcct ctttttgatc
  6721 gccaccattt gcatcgttat cctactttgg ggtatcaagg tggtttgaat ttgcttaatt
  6781 gggtagtaaa cactattttg gatgaacttg atcgcagtag caatatagcg ggtaaaactg
  6841 atatatcttt tgatttgatc cgttaatagg cggccgctcg ctaccttagg accgttatag
  6901 ttattaccct gttatcccta accggtggag gcttcttgtt atgacatgtt tttttggggt
  6961 acagtctatg cctcgggcat ccaagcagca agcgcgttac gccgtgggtc gatgtttgat
  7021 gttatggagc agcaacgatg ttacgcagca gggcagtcgc cctaaaacaa agttaaacat
  7081 catgggggaa gcggtgatcg ccgaagtatc gactcaacta tcagaggtag ttggcgtcat
  7141 cgagcgccat ctcgaaccga cgttgctggc cgtacatttg tacggctccg cagtggatgg
  7201 cggcctgaag ccacacagtg atattgattt gctggttacg gtgaccgtaa ggcttgatga
  7261 aacaacgcgg cgagctttga tcaacgacct tttggaaact tcggcttccc ctggagagag
  7321 cgagattctc cgcgctgtag aagtcaccat tgttgtgcac gacgacatca ttccgtggcg
  7381 ttatccagct aagcgcgaac tgcaatttgg agaatggcag cgcaatgaca ttcttgcagg
  7441 tatcttcgag ccagccacga tcgacattga tctggctatc ttgctgacaa aagcaagaga
  7501 acatagcgtt gccttggtag gtccagcggc ggaggaactc tttgatccgg ttcctgaaca
  7561 ggatctattt gaggcgctaa atgaaacctt aacgctatgg aactcgccgc ccgactgggc
  7621 tggcgatgag cgaaatgtag tgcttacgtt gtcccgcatt tggtacagcg cagtaaccgg
  7681 caaaatcgcg ccgaaggatg tcgctgccga ctgggcaatg gagcgcctgc cggcccagta
  7741 tcagcccgtc atacttgaag ctagacaggc ttatcttgga caagaagaag atcgcttggc
  7801 ctcgcgcgca gatcagttgg aagaatttgt ccactacgtg aaaggcgaga tcaccaaggt
  7861 agtcggcaaa taatctcgag ctcaagcttc gaattctgca gtcgacggta ccgcgggccc
  7921 gggatccacc tgatctagag tccgcaaaaa tcaccagtct ctctctacaa atctatctct
  7981 ctctattttt ctccagaata atgtgtgagt agttcccaga taagggaatt agggttctta
  8041 tagggtttcg ctcatgtgtt gagcatataa gaaaccctta gtatgtattt gtatttgtaa
  8101 aatacttcta tcaataaaat ttctaattcc taaaaccaaa atccagtgac gcggccgcat
  8161 taccctgtta tccctattaa ttaagagctc gctaccttaa gagaggatat cggcgcgccg
  8221 atcctagcct agtctatagg aggttttgaa aagaaaggag caataatcat tttcttgttc
  8281 tgtcaagagg gtgctattgc tcctttcttt tttttttttt ctttactaat ttcctagtat
  8341 tttactgaca tagacttttt tgtttacatt atcgaaaaag aaagagaggg tatttgcttg
  8401 catttattca tgatggatcc cccgggctgc aggcatgcaa gctaattccc gatctagtaa
  8461 catagatgac accgcgcgcc gcggaactag taattaattc ccgcctttcg ctttttgggg
  8521 gtggaaggca aaagaaaacg taggggaggg atagaatcac tacactatca cggccaacta
  8581 taccaactcc ttaatgtaag gatatattta atgctattta tgaaattcaa taataaaaag
  8641 aaatagtaaa aaaattactt tatcttggat cttgggcgga tagcgggaat cgaacccgca
  8701 tcttctcctt ggcaaagaga aattttacca ttcgaccata tccgcatttt tttgttcttg
  8761 atacacaata tgtacccaca tatatgatat ataaccggat cttatttgtg cagtgccggg
  8821 acacatattc tcttcggaac gattccaata attttgttaa ttatattctt tttattcaag
  8881 aagtttgacc cccctctaat ttttttgttt tctttatttg atttgcattt tctttgggga
  8941 cttagattca aatttaatgt gtctcacaac cgagaaaaat taggggggtc attttggttt
  9001 tgggtctgcg acgaataggt tcaagagatg agagaattaa ggatacccac cagaaagact
  9061 aatccaatcc ataaggaggt accagaaaat acaacatttt tgttacttga ccagccatca
  9121 ggagaagcaa atacaacggg tacgctaatc aataagatta atgaagtagc aattaatgca
  9181 aaaacagcca attggaaagc aagagtcatg cttttaatcc tccaagctac caacaaatga
  9241 actatatacc atttgatccc tctatcagcc aaaaaatatt aattgtgata aaatatgtca
  9301 tcgagggatt ttactttatc atgaatccat tgattctata tgacttatta ctactccccc
  9361 tttcgcactt tattcgtaca tggagtgggg tggggggaaa tggaattttc ttttttattt
  9421 cacaaatgga catgctagat catatatcta tatacggata gatagatcga tcggcggatt
  9481 cgcacctgag atctttctac agatagtggg ggtatccacc cctatagcca tgttctattc
  9541 ggaggaataa aataaaaata gtctttcgga gagatggctg agtggttgat agccccggtc
  9601 ttgaaaaccg gtatagtttt gaacaaagaa ctatcgaggg ttcgaatccc tctctctcct
  9661 tttttgctaa ttgaatagat ttttttattt agtggttttg cccaatctgc tatccgaaag
  9721 aaaagggaat ggctcggcta tcccacctag ccaagccaga aaaatagatt agatataaat
  9781 tagataaaat aaatgagttg aaaaaaaaaa aagaaaaaag gaatacttaa gctgattcca
  9841 agatgtatga ttgaatcaaa gtaatttgta cttcattcaa gcattggatc tcctgtctca
  9901 tatcaattaa gaggggtcat ggaaagaaca ggttcaaagt cgcgatcaat tcctttttca
  9961 aatcctgctg cagcgagctc cagcttttgt tccctttagt gagggttaat tgcgcgcttg
10021 gcgtaatcat ggtcatagct gtttcctgtg tgaaattgtt atccgctcac aattccacac
10081 aacatacgag ccggaagcat aaagtgtaaa gcctggggtg cctaatgagt gagctaactc
10141 acattaattg cgttgcgctc actgcccgct ttccagtcgg gaaacctgtc gtgccagctg
10201 cattaatgaa tcggccaacg cgcggggaga ggcggtttgc gtattgggcg ctcttccgct
10261 tcctcgctca ctgactcgct gcgctcggtc gttcggctgc ggcgagcggt atcagctcac
10321 tcaaaggcgg taatacggtt atccacagaa tcaggggata acgcaggaaa gaacatgtga
10381 gcaaaaggcc agcaaaaggc caggaaccgt aaaaaggccg cgttgctggc gtttttccat
10441 aggctccgcc cccctgacga gcatcacaaa aatcgacgct caagtcagag gtggcgaaac
10501 ccgacaggac tataaagata ccaggcgttt ccccctggaa gctccctcgt gcgctctcct
10561 gttccgaccc tgccgcttac cggatacctg tccgcctttc tcccttcggg aagcgtggcg
10621 ctttctcata gctcacgctg taggtatctc agttcggtgt aggtcgttcg ctccaagctg
10681 ggctgtgtgc acgaaccccc cgttcagccc gaccgctgcg ccttatccgg taactatcgt
10741 cttgagtcca acccggtaag acacgactta tcgccactgg cagcagccac tggtaacagg
10801 attagcagag cgaggtatgt aggcggtgct acagagttct tgaagtggtg gcctaactac
10861 ggctacacta gaaggacagt atttggtatc tgcgctctgc tgaagccagt taccttcgga
10921 aaaagagttg gtagctcttg atccggcaaa caaaccaccg ctggtagcgg tggttttttt
10981 gtttgcaagc agcagattac gcgcagaaaa aaaggatctc aagaagatcc tttgatcttt
11041 tctacggggt ctgacgctca gtggaacgaa aactcacgtt aagggatttt ggtcatgaga
11101 ttatcaaaaa ggatcttcac ctagatcctt ttaaattaaa aatgaagttt taaatcaatc
11161 taaagtatat atgagtaaac ttggtctgac agttaccaat gcttaatcag tgaggcacct
11221 atctcagcga tctgtctatt tcgttcatcc atagttgcct gactccccgt cgtgtagata
11281 actacgatac gggagggctt accatctggc cccagtgctg caatgatacc gcgagaccca
11341 cgctcaccgg ctccagattt atcagcaata aaccagccag ccggaagggc cgagcgcaga
11401 agtggtcctg caactttatc cgcctccatc cagtctatta attgttgccg ggaagctaga
11461 gtaagtagtt cgccagttaa tagtttgcgc aacgttgttg ccattgctac aggcatcgtg
11521 gtgtcacgct cgtcgtttgg tatggcttca ttcagctccg gttcccaacg atcaaggcga
11581 gttacatgat cccccatgtt gtgcaaaaaa gcggttagct ccttcggtcc tccgatcgtt
11641 gtcagaagta agttggccgc agtgttatca ctcatggtta tggcagcact gcataattct
11701 cttactgtca tgccatccgt aagatgcttt tctgtgactg gtgagtactc aaccaagtca
11761 ttctgagaat agtgtatgcg gcgaccgagt tgctcttgcc cggcgtcaat acgggataat
11821 accgcgccac atagcagaac tttaaaagtg ctcatcattg gaaaacgttc ttcggggcga
11881 aaactctcaa ggatcttacc gctgttgaga tccagttcga tgtaacccac tcgtgcaccc
11941 aactgatctt cagcatcttt tactttcacc agcgtttctg ggtgagcaaa aacaggaagg
12001 caaaatgccg caaaaaaggg aataagggcg acacggaaat gttgaatact catactcttc
12061 ctttttcaat attattgaag catttatcag ggttattgtc tcatgagcgg atacatattt
12121 gaatgtattt agaaaaataa acaaataggg gttccgcgca catttccccg aaaagtgcca
12181 c
//
```

TABLE 6
SEQ. ID. NO. 49

| | |
|---|---|
| PGE#0048 | 16791 bp DNA circular |
| SOURCE | Plant Genetic Engineering artificial |
| Host ORGANISM | E. coli/Agrobacterium |
| Target ORGANISM | Tomato or other plant Transient and stable transformation. |
| FEATURES | Location/Qualifiers |
| CDS | 12370 . . . 13338 |
| | spectinomycin\R |
| repeat_region | 13790 . . . 14070 |
| | ORI\E1 |
| rep_origin | complement(14880 . . . 15880) |
| | PVS1\rep |
| misc_feature | 1730..1797 |
| | RB |
| misc_feature | complement(11990 . . . 12033) |
| | LB |
| misc_recomb | 1828..3676 |
| | Reporter Nptll |
| CDS complement | (2544 . . . 3335) |
| | Nptll |
| CDS synthetic | DNA 9837 . . . 11591 |
| | TP-NifK912 |
| CDS synthetic DNA | 6961 . . . 8658 |
| | TP-NifD912 |
| CDS synthetic DNA | 4661 . . . 5770 |
| | TP-NifH912 |
| promoter | 3773 . . . 4660 |
| | 2X355 |
| terminator | 5774 . . . 6060 |
| | Trminator |
| promoter | 6073 . . . 6960 |
| | 2X355 |
| terminator | 8663 . . . 8939 |
| | Terminator |
| promoter | 8951 . . . 9836 |
| | 2X35S |
| ORIGIN | synthetic DNA |

```
        1 tgcgcgtgcc tttgatcgcc cgcgacacga caaaggccgc ttgtagcctt ccatccgtga
      61 cctcaatgcg ctgcttaacc agctccacca ggtcggcggt ggcccatatg tcgtaagggc
    121 ttggctgcac cggaatcagc acgaagtcgg ctgccttgat cgcggacaca gccaagtccg
    181 ccgcctgggg cgctccgtcg atcactacga agtcgcgccg gccgatggcc ttcacgtcgc
    241 ggtcaatcgt cgggcggtcg atgccgacaa cggttagcgg ttgatcttcc cgcacggccg
    301 cccaatcgcg ggcactgccc tggggatcgg aatcgactaa cagaacatcg gccccggcga
    361 gttgcagggc gcgggctaga tgggttgcga tggtcgtctt gcctgacccg cctttctggt
    421 taagtacagc gataaccttc atgcgttccc cttgcgtatt tgtttattta ctcatcgcat
    481 catatacgca gcgaccgcat gacgcaagct gttttactca aatacacatc acctttttag
    541 acggcggcgc tcggtttctt cagcggccaa gctggccggc caggccgcca gcttggcatc
    601 agacaaaccg gccaggattt catgcagccg cacggttgag acgtgcgcgg gcggctcgaa
    661 cacgtacccg gccgcgatca tctccgcctc gatctcttcg gtaatgaaaa acggttcgtc
    721 ctggccgtcc tggtgcggtt tcatgcttgt tcctcttggc gttcattctc ggcggccgcc
    781 agggcgtcgg cctcggtcaa tgcgtcctca cggaaggcac cgcgccgcct ggcctcggtg
    841 ggcgtcactt cctcgctgcg ctcaagtgcg cggtacaggg tcgagcgatg cacgccaagc
    901 agtgcagccg cctctttcac ggtgcggcct tcctggtcga tcagctcgcg ggcgtgcgcg
    961 atctgtgccg gggtgagggt agggcggggg ccaaacttca cgcctcgggc cttggcggcc
  1021 tcgcgcccgc tccgggtgcg gtcgatgatt agggaacgct cgaactcggc aatgccggcg
  1081 aacacggtca acaccatgcg gccggccggc gtggtggtgt cggcccacgg ctctgccagg
  1141 ctacgcaggc ccgcgccggc ctcctggatg cgctcggcaa tgtccagtag gtcgcgggtg
  1201 ctgcgggcca ggcggtctag cctggtcact gtcacaacgt cgccagggcg taggtggtca
  1261 agcatcctgg ccagctccgg gcggtcgcgc ctggtgccgg tgatcttctc ggaaaacagc
  1321 ttggtgcagc cggccgcgtg cagttcggcc cgttggttgg tcaagtcctg gtcgtcggtg
  1381 ctgacgcggg catagcccag caggccagcg gcggcgctct tgttcatggc gtaatgtctc
  1441 cggttctagt cgcaagtatt ctactttatg cgactaaaac acgcgacaag aaaacgccag
  1501 gaaaagggca gggcggcagc ctgtcgcgta acttaggact tgtgcgacat gtcgttttca
  1561 gaagacggct gcactgaacg tcagaagccg actgcactat agcagcggag gggttggatc
  1621 aaagtacttt gatcccgagg ggaaccctgt ggttggcatg cacatacaaa tggacgaacg
  1681 gataaacctt ttcacgccct tttaaatatc cgttattcta ataaacgctc ttttctctta
  1741 ggtttacccg ccaatatatc ctgtcaaaca ctgatagttt aaactgaagg cgggaaacga
  1801 caatctgatc caagctcaag ctaagcttca gagccaccac cctcagagcc gccaccagaa
  1861 ccaccaccag agccgccgcc agcattgaca ggaggcccga tctagtaaca tagatgacac
  1921 cgcgcgcgat aatttatcct agtttgcgcg ctatattttg ttttctatcg cgtattaaat
  1981 gtataattgc gggactctaa tcataaaaac ccatctcata aataacgtca tgcattacat
  2041 gttaattatt acatgcttaa cgtaattcaa cagaaattat atgataatca tcgcaagacc
  2101 ggcaacagga ttcaatctta agaaacttta ttgccaaatg tttgaacgat cggggatcat
  2161 ccgggtctgt ggcgggaact ccacgaaaat atccgaacgc agcaagatat cgcggtccat
  2221 ctcggtcttg cctgggcagt cgccgccgac gccgttgatg tggacgccgg gcccgatcat
  2281 attgtcgctc aggatcgtgg cgttgtgctt gtcggccgtt gctgtcgtaa tgatatcggc
  2341 accttcgacc gcctgttccg cagagatccc gtgggcgaag aactccagca tgagatcccc
  2401 gcgctggagg atcatccagc cggcgtcccg gaaaacgatt ccgaagccca acctttcata
  2461 gaaggcggcg gtggaatcga aatctcgtga tggcaggttg ggcgtcgctt ggtcggtcat
  2521 ttcgaacccc agagtcccgc tcagaagaac tcgtcaagaa ggcgatagaa ggcgatgcgc
  2581 tgcgaatcgg gagcggcgat accgtaaagc acgaggaagc ggtcagccca ttcgccgcca
  2641 agctcttcag caatatcacg ggtagccaac gctatgtcct gatagcggtc cgccacaccc
  2701 agccggccac agtcgatgaa tccagaaaag cggccatttt ccaccatgat attcggcaag
  2761 caggcatcgc catgggtcac gacgagatca tcgccgtcgg gcatgcgcgc cttgagcctg
  2821 gcgaacagtt cggctggcgc gagcccctga tgctcttcgt ccagatcatc ctgatcgaca
  2881 agaccggctt ccatccgagt acgtgctcgc tcgatgcgat gtttcgcttg gtggtcgaat
  2941 gggcaggtag ccggatcaag cgtatgcagc cgccgcattg catcagccat gatggatact
  3001 ttctcggcag gagcaaggtg agatgacagg agatcctgcc ccggcacttc gcccaatagc
  3061 agccagtccc ttcccgcttc agtgacaacg tcgagcacag ctgcgcaagg aacgcccgtc
  3121 gtggccagcc acgatagccg cgctgcctcg tcctgcagtt cattcagggc accggacagg
  3181 tcggtcttga caaaaagaac cgggcgcccc tgcgctgaca gccggaacac ggcggcatca
  3241 gagcagccga ttgtctgttg tgcccagtca tagccgaata gcctctccac ccaagcggcc
  3301 ggagaacctg cgtgcaatcc atcttgttca atcatgcgaa acgatccaga tccggtgcag
  3361 attatttgga ttgagagtga atatgagact ctaattggat accgagggga atttatggaa
  3421 cgtcagtgga gcatttttga caagaaatat ttgctagctg atagtgacct taggcgactt
  3481 ttgaacgcgc aataatggtt tctgacgtat gtgcttagct cattaaactc cagaaacccg
  3541 cggctgagtg gctccttcaa cgttgcggtt ctgtcagttc caaacgtaaa acggcttgtc
  3601 ccgcgtcatc ggcgggggtc ataacgtgac tcccttaatt ctccgctcat gatcagattg
  3661 tcgtttcccg ccttcaggta ccgcgatcgc tcgcgacctg caggcataaa gccgtcagtg
  3721 tccgcataaa gaaccaaccc ataataccca taatagctgt ttgccaaccg gtcaacatgt
  3781 ggagcacgac acacttgtct actccaaaaa tatcaaagat acagtctcag aagaccaaag
  3841 ggcaattgag acttttcaac aaagggtaat atccggaaac ctcctcggat tccattgccc
  3901 agctatctgt cactttattg tgaagatagt ggaaaaggaa ggtggctcct acaaatgcca
  3961 tcattgcgat aaaggaaagg ccatcgttga agatgcctct gccgacagtg gtcccaaaga
  4021 tggaccccca cccacgagga gcatcgtgga aaaagaagac gttccaacca cgtcttcaaa
  4081 gcaagtggat tgatgtgata acatggtgga gcacgacaca cttgtctact ccaaaaatat
  4141 caaagataca gtctcagaag accaaagggc aattgagact tttcaacaaa gggtaatatc
  4201 cggaaacctc ctcggattcc attgcccagc tatctgtcac tttattgtga agatagtgga
  4261 aaaggaaggt ggctcctaca aatgccatca ttgcgataaa ggaaaggcca tcgttgaaga
  4321 tgcctctgcc gacagtggtc ccaaagatgg acccccaccc acgaggagca tcgtggaaaa
  4381 agaagacgtt ccaaccacgt cttcaaagca agtggattga tgtgatatct ccactgacgt
  4441 aagggatgac gcacaatccc actatccttc gcaagaccct tcctctatat aaggaagttc
  4501 atttcatttg gagaggacgt cgagagttct caacacaaca tatacaaaac aaacgaatct
  4561 caagcaatca agcattctac ttctattgca gcaatttaaa tcatttcttt taaagcaaaa
  4621 gcaattttct gaaaattttc accatttacg aacgatagcc atggctagcg ctaccggact
  4681 cagatctatg gcttcctctg tcatttcttc agcagctgtt gccacacgca gcaatgttac
  4741 acaagctagc atggttgcac ctttcactgg tctcaaatct tcagccactt tccctgttac
  4801 aaagaagcaa aaccttgaca tcacttccat tgctagcaat ggtggaagag ttagctgcag
  4861 agctcaagct tcgaattcta tgtctatcga taagaaaatc cgtcaaatcg cattttacgg
  4921 aaaaggcggt atcggaaaat ccactacctc acaaaacact cttgctgcaa tggctgaaat
  4981 gggtcaaaga atccttatcg ttggatgcga tcctaaagca gacagtaccc gccttatgct
  5041 tcactctaaa gctcaaacca ccgtacttca tcttgcagct gaacgtggtg ctgttgaaga
  5101 tcttgaatta gaggaagtaa tgttaactgg ttttagagga gttaaatgcg tagagtctgg
  5161 tggtccagaa ccaggtgttg gatgtgcagg tcgaggaatc atcactgcta tcaatttctt
  5221 agaggaaaac ggtgcatatc aagatgtaga ttttgtttca tacgatgttt taggtgatgt
  5281 agtttgtggt ggattcgcta tgccgatccg tgagaacaaa gcacaagaaa tctatatcgt
  5341 aaccagtggt gagatgatgg ctatgtacgc agctaataac atcgctagag gtatcttaaa
  5401 gtatgcacat actggagggg ttcgattagg cgggttaatc tgtaattcac gtaacgtaga
  5461 tcgtgaaatc gagttgatcg aaactttggc taaaagattg aatactcaaa tgatccatta
  5521 cgttcctcgc gataacatcg tacaacatgc cgagttgcgt cgcatgacag taaatgaata
  5581 tgcccctgat tccaatcagt ctaatgagta tcgtatcttg gcgaataaaa tcatcaataa
  5641 tgaaaatttg aaagtaccta ctcctatcga gatggaagaa ctagaagaac tactgatcga
  5701 atttggtatc ctggaatctg aagaaaatgc tgctaaaatg atcgggactc ctgctcagtc
  5761 ttccactaaa taaggatcca cctgatctag agtccgcaaa aatcaccagt ctctctctac
  5821 aaatctatct ctctctattt ttctccagaa taatgtgtga gtagttccca gataagggaa
  5881 ttagggttct tatagggttt cgctcatgtg ttgagcatat aagaaaccct tagtatgtat
  5941 ttgtatttgt aaaatacttc tatcaataaa atttctaatt cctaaaacca aaatccagtg
  6001 acgcggccgc acccataata cccataatag ctgtttgcca tcgctacctt aggaccgtta
  6061 tagttaaccg gtcaacatgt ggagcacgac acacttgtct actccaaaaa tatcaaagat
  6121 acagtctcag aagaccaaag ggcaattgag acttttcaac aaagggtaat atccggaaac
  6181 ctcctcggat tccattgccc agctatctgt cactttattg tgaagatagt ggaaaaggaa
  6241 ggtggctcct acaaatgcca tcattgcgat aaaggaaagg ccatcgttga agatgcctct
  6301 gccgacagtg gtcccaaaga tggaccccca cccacgagga gcatcgtgga aaaagaagac
  6361 gttccaacca cgtcttcaaa gcaagtggat tgatgtgata acatggtgga gcacgacaca
  6421 cttgtctact ccaaaaatat caaagataca gtctcagaag accaaagggc aattgagact
  6481 tttcaacaaa gggtaatatc cggaaacctc ctcggattcc attgcccagc tatctgtcac
  6541 tttattgtga agatagtgga aaaggaaggt ggctcctaca aatgccatca ttgcgataaa
  6601 ggaaaggcca tcgttgaaga tgcctctgcc gacagtggtc ccaaagatgg acccccaccc
  6661 acgaggagca tcgtggaaaa agaagacgtt ccaaccacgt cttcaaagca agtggattga
  6721 tgtgatatct ccactgacgt aagggatgac gcacaatccc actatccttc gcaagaccct
  6781 tcctctatat aaggaagttc atttcatttg gagaggacgt cgagagttct caacacaaca
  6841 tatacaaaac aaacgaatct caagcaatca agcattctac ttctattgca gcaatttaaa
  6901 tcatttcttt taaagcaaaa gcaattttct gaaaattttc accatttacg aacgatagcc
  6961 atggctagcg ctaccggact cagatctatg gcttcctctg tcatttcttc agcagctgtt
  7021 gccacacgca gcaatgttac acaagctagc atggttgcac ctttcactgg tctcaaatct
  7081 tcagccactt tccctgttac aaagaagcaa aaccttgaca tcacttccat tgctagcaat
  7141 ggtggaagag ttagctgcag agctcaaatg actcctccag aaaatcaaaa cctagtagat
  7201 gagaataaag agttaatcaa agaagtgctt caagcttacc ctgaaaaggc acgaaaaaaa
  7261 agagagaagc atttaaatgt tcacgaagaa agtaaatccg attgtggtgt aaaatctaac
  7321 ataaagtcca ttccaggcgt tatgactgct cgtgggtgcg catatgctgg tagcaaaggg
  7381 gtagtttggg gtcctatcaa agatatgatt catatatccc acgggcctgt aggttgtggg
  7441 tactggtctt ggtccggaag acgtaattat tacgttggta tcaccggaat taactctttc
  7501 ggtactatgc attttacttc cgatttccag gaaagagaca tcgtatttgg aggtgataag
  7561 aaattgacca aaataattga tgagttagaa atcctttttc cattaaatcg tggagtgtct
  7621 attcaatcag aatgccctat cggtttgatt ggaggtgata tcgaagcagt tgctaaaaaa
  7681 gcatctaagc agtatggaaa accagtagtt cctttaagat gtgagggtct gcgtggagta
  7741 tcacaatctt taggtcatca cattgctaac gacgcaatca gagattggat tttcccagaa
  7801 tttgataagg ctaaaaaaaa taatactatc gactttgaac cttcaccgta cgatgttgca
  7861 cttattggag atcataacat cggtggagat gcttgggcat ctcgtatgtt attggaggaa
  7921 atgggtctga gagtagttgc tcaatggtca ggagatggta ctttaaatga acttattcaa
  7981 ggacctgcag ctaaattggt actgattcac tgctatcgtt ctatgaatta catctgtcgc
  8041 tcattggagg aacaatatgg tatgccgtgg atggaattca acttttttgg acctactaaa
  8101 attgcagctt ctcttcgtga gatcgcagct aaattcgaca gtaaaattca agaaaatgca
  8161 gaaaaagtta tcgctaaata caccccggta atgaatgcag tgcttgagaa atatcgccct
  8221 cgtttggaag gtaacactgt tatgctttac gtagggggtc ttcgccctcg tcatgttgta
  8281 cctgctttcg aagatttggg gattaaagtt gtaggtaccg ggtatgaatt tgcacataat
  8341 gatgattaca aacgcactac acactatatc gataacgcta ctattatcta cgatgatgtt
  8401 acagcctatg agttcgaaga atttgtaaaa gctaaaaaac ctgatcttat tgcctctggt
  8461 atcaaagaaa aatacgcttt tcagaaaatg ggtctacctt ttcgacaaat gcatagttgg
  8521 gattattctg gtccttatca tggttatgat cgttttgcca tctttgctcg agatatggat
  8581 cttgccctaa atagtcctac ttggagtcta atcggtgcgc cttggaaaaa agcggctact
  8641 aaagcgaaag ctgctgctta aggatccacc tgatctagag tccgcaaaaa tcaccagtct
  8701 ctctctacaa atctatctct ctctattttt ctccagaata atgtgtgagt agttcccaga
  8761 taagggaatt agggttctta tagggtttcg ctcatgtgtt gagcatataa gaaaccctta
  8821 gtatgtattt gtatttgtaa aatacttcta tcaataaaat ttctaattcc taaaaccaaa
  8881 atccagtgac gcggccgctc gctaccttag gaccgttata gttaattacc ctgttatccc
  8941 taaccggtca acatgtggag cacgacacac ttgtctactc caaaaatatc aaagatacag
  9001 tctcagaaga ccaaagggca attgagactt ttcaacaaag ggtaatatcc ggaaacctcc
  9061 tcggattcca ttgcccagct atctgtcact ttattgtgaa gatagtggaa aaggaaggtg
  9121 gctcctacaa atgccatcat tgcgataaag gaaaggccat cgttgaagat gcctctgccg
  9181 acagtggtcc caaagatgga cccccaccca cgaggagcat cgtggaaaaa gaagacgttc
  9241 caaccacgtc ttcaaagcaa gtggattgat gtgataacat ggtggagcac gacacacttg
  9301 tctactccaa aaatatcaaa gatacagtct cagaagacca aagggcaatt gagacttttc
  9361 aacaaagggt aatatccgga aacctcctcg gattccattg cccagctatc tgtcacttta
  9421 ttgtgaagat agtggaaaag gaaggtggct cctacaaatg ccatcattgc gataaaggaa
  9481 aggccatcgt tgaagatgcc tctgccgaca gtggtcccaa agatggaccc ccacccacga
  9541 ggagcatcgt ggaaaaagaa gacgttccaa ccacgtcttc aaagcaagtg gattgatgtg
  9601 atatctccac tgacgtaagg gatgacgcac aatcccacta tccttcgcaa gacccttcct
  9661 ctatataagg aagttcattt catttggaga ggacgtcgag agttctcaac acaacatata
  9721 caaaacaaac gaatctcaag caatcaagca ttctacttct attgcagcaa tttaaatcat
  9781 ttcttttaaa gcaaaagcaa ttttctgaaa attttcacca tttacgaacg atagccatgg
  9841 ctagcgctac cggactcaga tctatggctt cctctgtcat ttcttcagca gctgttgcca
  9901 cacgcagcaa tgttacacaa gctagcatgg ttgcaccttt cactggtctc aaatcttcag
  9961 ccactttccc tgttacaaag aagcaaaacc ttgacatcac ttccattgct agcaatggtg
10021 gaagagttag ctgcagagct caagcttcga attctatgcc tcaaaatcca gagaaaattg
10081 tagatcatgt acaattattt cagcaacctg aatatcaaga acttttcaaa aataaacacg
10141 aacaatttga gggagctcat tccccagaag aagtacagag agtatccgaa tggactaaag
10201 gttgggagta cagagaaaag aactttgcac gtcaagcttt aacagtaaat cctgcaaaag
10261 gatgccaacc agtaggtgct atcttcgcag ctgtaggatt tgaaggtact ctacctttcg
10321 tacaaggatc ccagggttgt gtagcatatt ttagaacaca tttatcccgt cactacaaag
10381 aaccattctc tgctgtatcc tcatctatga ctgaggatgc agctgtattt ggaggtctta
10441 acaatatgat tgaaggatta caagtatcat atcaactata caaacctaaa atgatcgcag
10501 tatgtacaac ttgtatggct gaagtaattg gtgatgattt aggagcattc atcacaaatg
10561 ctaaaaacgc aggttctatt ccacaagatt ttcctgtacc attcgctcat actccttcat
10621 ttgtaggatc tcatacaacc ggttatgata atatgatgaa gggaatcctt tcaaatttaa
10681 ctgagggtaa aaaaaaagat aagaccaacg gaaaaattaa tttcatccca ggttttgata
10741 cttacgtagg aaataacaga gaactgaaac gtatgttagg tcttatggga attgattata
10801 ccatcttagc agataattct gattacctgg atgctcctaa tactggtgaa tatgatatgt
10861 acccgggcgg taccaaatta gaggatgcag ctgattcatg caacgcagaa gctactgtag
10921 cccttcagag atataccact accaagactc gagaatacat tgagaccaaa tggaaacaac
10981 aaactaaagt attacgtcct ttcggggtaa aaggtaccga cgaatttctt atgactttat
11041 ctgaaatgac cgggaaaccg atccctcaag aacttgagga tgaacgaggt cgtttagtag
11101 acgctatgac tgatagttat gcctggattc acgggaaaaa attcgctatc tacggtgatc
11161 cggaccttat ttattctgta accagttttt tacttgaatt aggggccgaa cctgtacata
11221 tcctttgtaa taatggtgat gaagagttca aaaaagagat ggaagctatt ttatctgcga
11281 gtccgtttgg gaaacaagct actgtatgga tccagaaaga tttgtggcat cttcgatctt
11341 tgctttttac tgaacctgta gatttttttg taggtaacag ttacgggaaa tatttgtggc
11401 gcgacaccaa aattcctatg gtacgtatcg gttatcctct ttttgatcgc caccatttgc
11461 atcgttatcc tactttgggg tatcaaggtg gtttgaattt gcttaattgg gtagtaaaca
11521 ctattttgga tgaacttgat cgcagtagca atatagcggg taaaactgat atatcttttg
11581 atttgatccg ttaagatcca cctgatctag agtccgcaaa aatcaccagt ctctctctac
11641 aaatctatct ctctctattt ttctccagaa taatgtgtga gtagttccca gataagggaa
11701 ttagggttct tatagggttt cgctcatgtg ttgagcatat aagaaaccct tagtatgtat
11761 ttgtatttgt aaaatacttc tatcaataaa atttctaatt cctaaaacca aaatccagtg
11821 acgcggccgc attaccctgt tatccctatt aattaagagc tcgctacctt aagagaggat
11881 atcggcgcgc ctctagaatt taaatggatc ctacgtactc gaggaattca attcggcgtt
11941 aattcagtac attaaaaacg tccgcaatgt gttattaagt tgtctaagcg tcaatttgtt
12001 tacaccacaa tatatcctgc caccagccag ccaacagctc cccgaccggc agctcggcac
12061 aaaatcacca ctcgatacag gcagcccatc agtccgggac ggcgtcagcg ggagagccgt
12121 tgtaaggcgg cagactttgc tcatgttacc gatgctattc ggaagaacgg caactaagct
12181 gccgggtttg aaacacggat gatctcgcgg agggtagcat gttgattgta acgatgacag
12241 agcgttgctg cctgtgatca attcgggcac gaacccagtg gacataagcc tcgttcggtt
12301 cgtaagctgt aatgcaagta gcgtaactgc cgtcacgcaa ctggtccaga accttgaccg
12361 aacgcagcgg tggtaacggc gcagtggcgg ttttcatggc ttcttgttat gacatgtttt
12421 tttggggtac agtctatgcc tcgggcatcc aagcagcaag cgcgttacgc cgtgggtcga
12481 tgtttgatgt tatggagcag caacgatgtt acgcagcagg gcagtcgccc taaaacaaag
12541 ttaaacatca tgggggaagc ggtgatcgcc gaagtatcga ctcaactatc agaggtagtt
12601 ggcgtcatcg agcgccatct cgaaccgacg ttgctggccg tacatttgta cggctccgca
12661 gtggatggcg gcctgaagcc acacagtgat attgatttgc tggttacggt gaccgtaagg
12721 cttgatgaaa caacgcggcg agctttgatc aacgaccttt tggaaacttc ggcttcccct
12781 ggagagagcg agattctccg cgctgtagaa gtcaccattg ttgtgcacga cgacatcatt
12841 ccgtggcgtt atccagctaa gcgcgaactg caatttggag aatggcagcg caatgacatt
12901 cttgcaggta tcttcgagcc agccacgatc gacattgatc tggctatctt gctgacaaaa
12961 gcaagagaac atagcgttgc cttggtaggt ccagcggcgg aggaactctt tgatccggtt
13021 cctgaacagg atctatttga ggcgctaaat gaaaccttaa cgctatggaa ctcgccgccc
13081 gactgggctg gcgatgagcg aaatgtagtg cttacgttgt cccgcatttg gtacagcgca
13141 gtaaccggca aaatcgcgcc gaaggatgtc gctgccgact gggcaatgga gcgcctgccg
13201 gcccagtatc agcccgtcat acttgaagct agacaggctt atcttggaca agaagaagat
13261 cgcttggcct cgcgcgcaga tcagttggaa gaatttgtcc actacgtgaa aggcgagatc
13321 accaaggtag tcggcaaata atgtctagct agaaattcgt tcaagccgac gccgcttcgc
13381 cggcgttaac tcaagcgatt agatgcacta agcacataat tgctcacagc caaactatca
13441 ggtcaagtct gcttttatta tttttaagcg tgcataataa gccctacaca aattgggaga
13501 tatatcatgc atgaccaaaa tcccttaacg tgagttttcg ttccactgag cgtcagaccc
13561 cgtagaaaag atcaaaggat cttcttgaga tccttttttt ctgcgcgtaa tctgctgctt
13621 gcaaacaaaa aaaccaccgc taccagcggt ggtttgtttg ccggatcaag agctaccaac
13681 tctttttccg aaggtaactg gcttcagcag agcgcagata ccaaatactg tccttctagt
13741 gtagccgtag ttaggccacc acttcaagaa ctctgtagca ccgcctacat acctcgctct
13801 gctaatcctg ttaccagtgg ctgctgccag tggcgataag tcgtgtctta ccgggttgga
13861 ctcaagacga tagttaccgg ataaggcgca gcggtcgggc tgaacggggg gttcgtgcac
13921 acagcccagc ttggagcgaa cgacctacac cgaactgaga tacctacagc gtgagctatg
13981 agaaagcgcc acgcttcccg aagggagaaa ggcggacagg tatccggtaa gcggcagggt
14041 cggaacagga gagcgcacga gggagcttcc agggggaaac gcctggtatc tttatagtcc
14101 tgtcgggttt cgccacctct gacttgagcg tcgatttttg tgatgctcgt caggggggcg
14161 gagcctatgg aaaaacgcca gcaacgcggc ctttttacgg ttcctggcct tttgctggcc
14221 ttttgctcac atgttctttc ctgcgttatc ccctgattct gtggataacc gtattaccgc
14281 ctttgagtga gctgataccg ctcgccgcag ccgaacgacc gagcgcagcg agtcagtgag
14341 cgaggaagcg gaagagcgcc tgatgcggta ttttctcctt acgcatctgt gcggtatttc
14401 acaccgcata tggtgcactc tcagtacaat ctgctctgat gccgcatagt taagccagta
14461 tacactccgc tatcgctacg tgactgggtc atggctgcgc cccgacaccc gccaacaccc
14521 gctgacgcgc cctgacgggc ttgtctgctc ccggcatccg cttacagaca agctgtgacc
14581 gtctccggga gctgcatgtg tcagaggttt tcaccgtcat caccgaaacg cgcgaggcag
14641 ggtgccttga tgtgggcgcc ggcggtcgag tggcgacggc gcggcttgtc cgcgccctgg
14701 tagattgcct ggccgtaggc cagccatttt tgagcggcca gcggccgcga taggccgacg
14761 cgaagcggcg gggcgtaggg agcgcagcga ccgaagggta ggcgcttttt gcagctcttc
14821 ggctgtgcgc tggccagaca gttatgcaca ggccaggcgg gttttaagag ttttaataag
14881 ttttaaagag ttttaggcgg aaaaatcgcc ttttttctct tttatatcag tcacttacat
14941 gtgtgaccgg ttcccaatgt acggctttgg gttcccaatg tacgggttcc ggttcccaat
15001 gtacggcttt gggttcccaa tgtacgtgct atccacagga aagagacctt ttcgaccttt
15061 ttcccctgct agggcaattt gccctagcat ctgctccgta cattaggaac cggcggatgc
15121 ttcgccctcg atcaggttgc ggtagcgcat gactaggatc gggccagcct gccccgcctc
15181 ctccttcaaa tcgtactccg gcaggtcatt tgacccgatc agcttgcgca cggtgaaaca
15241 gaacttcttg aactctccgg cgctgccact gcgttcgtag atcgtcttga acaaccatct
15301 ggcttctgcc ttgcctgcgg cgcggcgtgc caggcggtag agaaaacggc cgatgccggg
15361 atcgatcaaa aagtaatcgg ggtgaaccgt cagcacgtcc gggttcttgc cttctgtgat
15421 ctcgcggtac atccaatcag ctagctcgat ctcgatgtac tccggccgcc cggtttcgct
15481 ctttacgatc ttgtagcggc taatcaaggc ttcaccctcg gataccgtca ccaggcggcc
15541 gttcttggcc ttcttcgtac gctgcatggc aacgtgcgtg gtgtttaacc gaatgcaggt
15601 ttctaccagg tcgtctttct gctttccgcc atcggctcgc cggcagaact tgagtacgtc
15661 cgcaacgtgt ggacggaaca cgcggccggg cttgtctccc ttcccttccc ggtatcggtt
15721 catggattcg gttagatggg aaaccgccat cagtaccagg tcgtaatccc acacactggc
15781 catgccggcc ggccctgcgg aaacctctac gtgcccgtct ggaagctcgt agcggatcac
15841 ctcgccagct cgtcggtcac gcttcgacag acggaaaacg gccacgtcca tgatgctgcg
15901 actatcgcgg gtgcccacgt catagagcat cggaacgaaa aaatctggtt gctcgtcgcc
15961 cttgggcggc ttcctaatcg acggcgcacc ggctgccggc ggttgccggg attctttgcg
16021 gattcgatca gcggccgctt gccacgattc accggggcgt gcttctgcct cgatgcgttg
16081 ccgctgggcg gcctgcgcgg ccttcaactt ctccaccagg tcatcaccca gcgccgcgcc
16141 gatttgtacc gggccggatg gtttgcgacc gtcacgccga ttcctcgggc ttgggggttc
16201 cagtgccatt gcagggccgg cagacaaccc agccgcttac gcctggccaa ccgcccgttc
16261 ctccacacat ggggcattcc acggcgtcgg tgcctggttg ttcttgattt tccatgccgc
16321 ctcctttagc cgctaaaatt catctactca tttattcatt tgctcattta ctctggtagc
16381 tgcgcgatgt attcagatag cagctcggta atggtcttgc cttggcgtac cgcgtacatc
16441 ttcagcttgg tgtgatcctc cgccggcaac tgaaagttga cccgcttcat ggctggcgtg
16501 tctgccaggc tggccaacgt tgcagccttg ctgctgcgtg cgctcggacg gccggcactt
16561 agcgtgtttg tgcttttgct cattttctct ttacctcatt aactcaaatg agttttgatt
16621 taatttcagc ggccagcgcc tggacctcgc gggcagcgtc gccctcgggt tctgattcaa
16681 gaacggttgt gccggcggcg gcagtgcctg ggtagctcac gcgctgcgtg atacgggact
16741 caagaatggg cagctcgtac ccggccagcg cctcggcaac ctcaccgccg t
//
```

It will be understood that various modifications may be made to the procedures and embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A transgenic plant exhibiting enhanced nitrogen fixatation produced by a method comprising the steps of:
contacting one or more plant cells with a recombinant nucleic acid sequence encoding Nif-H, Nif-D and Nif-K genes operatively linked to a promoter sequence and a terminator sequence;
regenerating one or more plants from the plant cells; and
selecting one or more plants, cultivated from the plant cells, exhibiting enhanced nitrogen fixation, wherein said one or more plants comprise the recombinant nucleic acid sequence encoding the Nif-H, Nif-D and Nif-K genes.

2. Seeds, stems, leaves, or roots of the transgenic plant of claim 1, wherein the seeds, stems, leaves or roots comprise the recombinant nucleic acid sequence encoding the Nif-H, Nif-D and Nif-K genes.

3. A transgenic plant as recited in claim 1, wherein the transgenic plant is selected from a group consisting of canola, corn, rice, tobacco, soybean, cotton, alfalfa, tomato, wheat, potato and barley,

4. A transgenic plant as recited in claim 3, wherein the transgenic plant is tomato or tobacco.

5. A progeny of the transgenic plant of claim 1, wherein said progeny comprise the recombinant nucleic acid sequence encoding the Nif-H, Nif-D and Nif-K genes.

6. A progeny of the plant of claim 3, wherein said progeny comprise the recombinant nucleic acid sequence encoding the Nif-H, Nif-D and Nif-K genes.

7. A progeny of the plant of claim 4, wherein said progeny comprise the recombinant nucleic acid sequence encoding the Nif-H, Nif-D and Nif-K genes.

8. A transgenic plant as recited in claim 1, wherein the step of selecting one or more plants comprises growing the one or more plants under conditions of low nitrogen input.

9. A transgenic plant as recited in claim 1, wherein the recombinant nucleic acid sequence encoding the Nif-H, Nif-D and Nif-K genes that contacts the one or more cells is obtained from a source selected from a group consisting of cyanobacteria, azotobacteraceae, rhizobia, and frankia.

10. A method for reducing the overall concentration of nitrogen in soil comprising:
placing the transgenic plant of claim 1 in contact with the soil so that as the transgenic plant grows nitrogen is used from the soil thereby reducing the overall concentration of nitrogen in the soil.

11. A transgenic plant exhibiting enhanced nitrogen fixatation produced by a method comprising the steps of:
contacting a plant cell with a recombinant nucleic acid sequence encoding one of SEQ. ID. NO. 1, SEQ. ID. NO. 2 and SEQ. ID. NO. 3 to produce a transgenic plant;
regenerating one or more transgenic plants from the plant cell; and
selecting one or more transgenic plants cultivated from the plant cell, wherein the selected transgenic plants each exhibit enhanced nitrogen fixation,
wherein the selected one or more transgenic plants each comprise the recombinant nucleic acid sequence encoding SEQ. ID. No. 1, SEQ. ID. No. 2 or SEQ. ID. NO. 3.

12. Seeds, stems, leaves or roots of the transgenic plant of claim 11, wherein the seeds, stems, leaves or roots comprise the recombinant nucleic acid sequence encoding one of SEQ. ID. NO. 1, SEQ. ID. NO. 2 and SEQ. ID. NO. 3.

13. A transgenic plant as recited in claim 11, wherein the transgenic plant is selected from the group consisting of canola, corn, rice, tobacco, soybean, cotton, alfalfa, tomato, wheat, potato and barley.

14. A plant-derived commercial product, which is derived from a transgenic plant produced according to the method of claim 11, wherein said transgenic plant comprises a recombinant nucleic acid sequence encoding one of SEQ. ID. NO. 1, SEQ. ID. NO. 2 and SEQ. ID. NO. 3.

15. A plant-derived commercial product as recited in claim 14, wherein said commercial product is pulp, paper, a paper product, lumber, cigarette, cigar, chewing tobacco, bread, flour, cereal, oat meal, or rice.

16. A method for reducing the overall concentration of nitrogen in soil comprising:
placing the transgenic plant of claim 11 in contact with the soil so that as the plant grows nitrogen is used from the soil thereby reducing the overall concentration of nitrogen in the soil.

17. A transgenic plant exhibiting enhanced nitrogen fixatation produced by a method comprising the steps of:
contacting a plant cell with a recombinant nucleic acid sequence encoding one of SEQ. ID. NO. 1, SEQ. ID. NO. 2 and SEQ. ID. NO. 3 to produce a transgenic plant;
contacting the plant cell with at least one synthetic coding DNA sequence (sCDS) optimized for tomato chloroplast expression;
regenerating one or more transgenic plants from the plant cell; and
selecting one or more transgenic plants, cultivated from the plant cell exhibiting enhanced nitrogen fixation, wherein said one or more plants comprise the recombinant nucleic acid sequence encoding SEQ. ID. NO. 1, SEQ. ID. NO. 2 or SEQ. ID. NO. 3 and the sCDS.

18. A transgenic plant as recited claim 17, wherein the transgenic plant is selected from the group consisting of canola, corn, rice, tobacco, soybean, cotton, alfalfa, tomato, wheat, potato and barley.

19. A transgenic plant as recited in claim 17, wherein the sCDS comprises one of a group consisting essentially of SEQ. ID. NO. 29, SEQ. ID. NO. 30, SEQ. ID. NO. 31, SEQ. ID. NO. 32, SEQ. ID. NO. 33, SEQ. ID. NO. 34, SEQ. ID. NO 35, SEQ. ID. NO. 36, SEQ. ID. NO. 37, SEQ. ID. NO. 38, SEQ. ID. NO. 39 and SEQ. ID. NO. 40.

20. A method for reducing the overall concentration of nitrogen in soil comprising:
placing the transgenic plant of claim 17 in contact with the soil so that as the plant grows nitrogen is used from the soil thereby reducing the overall concentration of nitrogen in the soil.

21. A method for increasing nitrogen concentration in soil comprising:
placing the transgenic plant of claim 17 in contact with soil so that the plant releases nitrogen into the soil as the plant decays.

22. A transgenic plant exhibiting enhanced nitrogen fixatation produced by a method comprising the steps of:
contacting a plant cell with a recombinant nucleic acid sequence encoding one of SEQ. ID. NO. 46, SEQ. ID. NO. 47, SEQ. ID. NO. 48 and SEQ. ID. NO. 49 to produce a transgenic plant;
regenerating one or more transgenic plants from the plant cell; and
selecting one or more transgenic plants cultivated from the plant cell, wherein the selected plants each exhibit enhanced nitrogen fixation,
wherein the selected one or more transgenic plants each comprise the recombinant nucleic acid sequence encoding SEQ. ID. NO. 46, SEQ. ID. NO. 47, SEQ. ID. NO. 48 or SEQ. ID. NO. 49.

23. Seeds, stems, leaves or roots of the transgenic plant of claim 22, wherein the seeds, stems, leaves or roots comprise the recombinant nucleic acid sequence encoding one of SEQ. ID. NO. 46, SEQ. ID. NO. 47, SEQ. ID. NO. 48 and SEQ. ID. NO. 49.

24. A transgenic plant as recited in claim 22, wherein the transgenic plant is selected from the group consisting of canola, corn, rice, tobacco, soybean, cotton, alfalfa, tomato, wheat, potato and barley.

25. A transgenic plant-derived commercial product, which is derived from a transgenic plant produced according to the method of claim 22, wherein said transgenic plant comprises a recombinant nucleic acid sequence encoding one of SEQ. ID. NO. 46, SEQ. ID. NO. 47, SEQ. ID. NO. 48 and SEQ. ID. NO. 49.

26. A transgenic plant-derived commercial product as recited in claim 25, wherein said commercial product is pulp, paper, a paper product, lumber, cigarette, cigar, chewing tobacco, bread, flour, cereal, oat meal, or rice.

27. A method for reducing the overall concentration of nitrogen in soil comprising:
placing the transgenic plant of claim 22 in contact with the soil so that as the plant grows nitrogen is used from the soil thereby reducing the overall concentration of nitrogen in the soil.

28. A transgenic plant exhibiting enhanced nitrogen fixatation produced by a method comprising the steps of:
contacting one or more transgenic plant cells with a recombinant nucleic acid sequence encoding a Nif-H gene operatively linked to a first promoter;
contacting the transgenic plant cells with a recombinant nucleic acid sequence encoding a Nif-D gene operatively linked to a second promoter;
contacting the transgenic plant cells with a recombinant nucleic acid sequence encoding a Nif-K gene operatively linked to a third promoter;
regenerating one or more transgenic plants from the plant cells; and
selecting one or more transgenic plants cultivated from the plant cells, wherein the selected transgenic plants each exhibit enhanced nitrogen fixation,
wherein the selected transgenic plants each comprise the recombinant nucleic acid sequence encoding the Nif-H gene, the recombinant nucleic acid sequence encoding the Nif-D gene and the recombinant nucleic acid sequence encoding the Nif-K gene.

29. A transgenic plant as recited in claim 28, wherein the recombinant nucleic acid sequence encoding the Nif-H gene is selected from a group consisting essentially of SEQ. ID. NOs. 29, 32, 35 and 38.

30. A transgenic plant as recited in claim 28, wherein the recombinant nucleic acid sequence encoding the Nif-D gene is selected from a group consisting essentially of SEQ. ID. NOs. 30, 33, 36 and 39.

31. A transgenic plant as recited in claim 28, wherein the recombinant nucleic acid sequence encoding the Nif-K gene is selected from a group consisting essentially of SEQ. ID. NOs. 31, 34, 37 and 40.

32. A transgenic plant as recited in claim 28, wherein the first promoter is selected from a group consisting essentially of SEQ. ID. NOs. 41-45.

33. A transgenic plant as recited in claim 28, wherein the second promoter is selected from a group consisting essentially of SEQ. ID. NOs. 41-45.

34. A transgenic plant as recited in claim 28, wherein the third promoter is selected from a group consisting essentially of SEQ. ID. NOs. 41-45.

35. A transgenic plant as recited in claim 28, wherein at least one of the promoters comprises SEQ. ID. NO. 4 or SEQ. ID. NO. 28.
